(19) United States
(12) Patent Application Publication
(10) Pub. No.: US 2023/0373937 A1
(43) Pub. Date: Nov. 23, 2023

(54) INHIBITORS OF SPINSTER HOMOLOG 2 (SPNS2) FOR USE IN THERAPY

(71) Applicants: University of Virginia Patent Foundation, Charlottesville, VA (US); Virginia Tech Intellectual Properties, Inc., Blacksburg, VA (US)

(72) Inventors: Kevin R. Lynch, Charlottesville, VA (US); Yugesh Kharel, Charlottesville, VA (US); Webster L. Santos, Blacksburg, VA (US); Russell G. Fritzemeier, Atlanta, GA (US); Ashley N. Peralta, Los Alamos, NM (US)

(21) Appl. No.: 18/044,688

(22) PCT Filed: Sep. 8, 2021
(86) PCT No.: PCT/US2021/049531

§ 371 Date: Mar. 9, 2023

Related U.S. Application Data
(60) Provisional application No. 63/076,111, filed on Sep. 9, 2020.

(51) Int. Cl. C07D 271/06; C07D 413/04

(52) U.S. Cl. CPC C07D 271/06; C07D 413/04

(57) ABSTRACT

The present disclosure provides SPNS2 inhibitor compounds according to Formula (I) and their pharmaceutically acceptable salts, and/or tautomers as described in the disclosure, and the disclosure provides their pharmaceutical compositions and methods of use in therapy.

### PRIORITY APPLICATION

This application claims priority to U.S. Patent Application Ser. No. 63/076,111, filed Sep. 9, 2020, the disclosure of which is incorporated herein in its entirety by reference.

### STATEMENT REGARDING FEDERALLY SPONSORED RESEARCH OR DEVELOPMENT

This invention was made with government support under R01GM121075 and R01AI144026 awarded by the National Institutes of Health. The government has certain rights in the invention.

### BACKGROUND

Sphingosine 1-phosphate (S1P) is a simple lipid that is chemotactic when present extracellularly, but which is also a second messenger when intracellular. These roles require compartmentalization, which is provided in part by extrusion of S1P from cells. The S1P transporters, SPNS2 (endothelium) and MFSD2B (erythrocytes), release S1P from cells. When this release is coupled with S1P degradation in tissue parenchyma, a differential is generated between the extracellular (high) and intracellular (low) S1P concentrations. Mouse genetic studies indicate that endothelial cells use SPNS2 to provide most of the S1P in lymph as well as about one-third of plasma S1P, whereas erythrocytes provide the remainder of plasma S1P via MFSD2B. The S1P gradient in blood functions both to maintain endothelial barrier integrity and promote migration of lymphocytes from the thymus to the blood.

The lymph S1P gradient is particularly important for egress of lymphocytes from secondary lymphoid tissue into efferent lymph for correct temporal and spatial positioning of immune cells. However, on-target agonist activity at endothelial and cardiac S1P receptors drives adverse events such as first dose bradycardia.

### SUMMARY

The present disclosure provides, in various embodiments, compounds and their pharmaceutically acceptable salts conforming to Formula I as SPNS2 inhibitors that avoid on-target adverse activity:

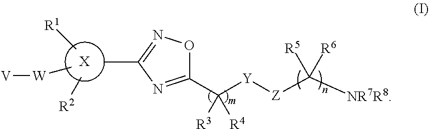

X is a C₆-C₁₀-aryl or 5- to 10-membered heteroaryl (wherein 1 to 4 heteroaryl ring members are independently selected from N, O, and S).

R¹ and R² are independently selected from the group consisting of H, C₁-C₆₋alkyl, C₁-C₆₋alkoxy, C₁₋₆₋haloalkoxy, C₃-C₈-cycloalkyl, C₁-C₆-haloalkyl, CN, halo, and —C(O)N(H)(C₁-C₆₋alkyl).

W is a bond, O, NH, —NHC(O)—, or —O—(N═)C(R)— (wherein R is H or C₁-C₆-alkyl).

V is selected from the group consisting of H, C₁-C₁₄-alkyl, C₂-C₁₂-alkenyl, (C₆-C₁₀)aryl, (C₆-C₁₀)heteroaryl, —C₁-C₁₀-alkyl-(C₆-C₁₀)aryl, —C₂-C₁₂-alkenyl-(C₆-C₁₀)aryl, —C₁-C₁₀-alkyl-(C₃-C₈)cycloalkyl, -(3- to 14-membered heterocycloalkyl) (wherein 1-4 heterocycloalkyl members are independently selected from N, O, and S), —(C₁-C₁₀)alkyl-(3- to 14-membered heterocycloalkyl) (wherein 1-4 heterocycloalkyl members are independently selected from N, O, and S).

Y is selected from a bond, —NH—, (C₆-C₁₀)arylenyl, and (C₃-C₈)cycloalkylenyl.

Z is selected from a bond and —C(O)—.

R³ and R⁴ are independently selected from the group consisting of, H, C₁-C₆-alkyl, OH, C₁-C₆₋alkoxy, halo, —NRR′, —C(O)R, and —C(O)OR, wherein R and R′ are independently selected from H and C₁-C₆-alkyl.

R⁵ and R⁶ are independently selected from the group consisting of, H, C₁-C₆-alkyl, OH, C₁-C₆₋alkoxy, halo, —C(O)R, and —C(O)OR, wherein R is H or C₁-C₆-alkyl, or R⁵ and R⁶, together with the carbon to which they are bound, form a —(C₃-C₈)cycloalkyl.

Subscript m is an integer selected from 0, 1, 2, 3, 4, 5, and 6.

Subscript n is an integer selected from 0, 1, and 2.

R⁷ and R⁸ are independently selected from the group consisting of H and C₁-C₆-alkyl, or R⁷ and R⁸, together with the nitrogen atom to which they are bound, form a 5- to 7-membered heterocycloalkyl (wherein 1-4 other heterocycloalkyl members are optionally independently selected from NH, 0, and S).

Optionally, in some embodiments, one of R⁵ and R⁶ and one of R⁷ and R⁸, together with the carbon and nitrogen atoms to which they are bound, respectively, form a 4- to 7-membered heterocycloalkyl (wherein 1-4 other heterocycloalkyl members are optionally independently selected from NH, 0, and S).

Each alkyl, alkoxy, alkenyl, alkynyl, aryl, cycloalkyl, heterocycloalkyl, and heteroaryl is optionally substituted with 1-5 substituents independently selected from the group consisting of hydroxy, halo, C₁-C₆₋haloalkoxy, C₁-C₆-haloalkyl, —NR″₂, —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl).

Each R″ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S.

Another embodiment of the disclosure is a pharmaceutical composition comprising a compound of Formula I or a pharmaceutically acceptable salt thereof.

The disclosure also provides, in an embodiment, a method of inhibiting spinster homolog 2 (SPNS2), comprising contacting SPNS2 with an effective amount of a compound of Formula I or a pharmaceutically acceptable salt thereof.

Still another embodiment is a method of treating a patient afflicted by a neoplastic disease, comprising administering to the patient a therapeutically effective amount of a compound of Formula I or a pharmaceutically acceptable salt thereof.

In an embodiment, the disclosure provides a method of treating a patient afflicted with an allergic disease, comprising administering to the patient a therapeutically effective amount of a compound of Formula I or a pharmaceutically acceptable salt thereof.

In another embodiment, the disclosure provides a method of treating a patient afflicted with an autoimmune disease, comprising administering to the patient a therapeutically effective amount of a compound of Formula I or a pharmaceutically acceptable salt thereof.

### DETAILED DESCRIPTION

In a properly functioning immune system, the proper cells get to the proper places at the proper times. Gradients of the chemotactic lipid, sphingosine 1-phosphate (S1P), enable correct positioning of immune cells; lymphocyte egress from secondary lymphoid tissues is particularly dependent on S1P signaling. S1P's role in lymphocyte trafficking was discovered when the mechanism of action of the immunosuppressive drug fingolimod (FTY720) was investigated. Fingolimod's active metabolite, phospho-FTY720, desensitizes lymphocyte S1P1 receptors; thereby, rendering these cells unable to detect the S1P-rich environment of efferent lymph¹. The resulting lymphopenia is now recognized as a general property of S1P1 receptor agonists. Although fingolimod eventually became a medicine for treating relapsing remitting multiple sclerosis², S1P1 receptor agonists have several on target liabilities including initial dose bradycardia and compromised endothelial barrier function². Therefore, alternative strategies to achieve immunosuppression by modulating S1P signaling without undesirable on-target activity are needed.

S1P is synthesized ubiquitously, but its intracellular accumulation is limited by degradation and export. In lymph nodes (LN), brisk catabolic activity by S1P lyase keeps S1P³ low while lymph endothelial cells extrude S1P into lymph via a transporter, SPNS2⁴, resulting in a lymph-LN S1P gradient. Vascular (blood) S1P gradients are likewise maintained by prominent S1P catabolic activity in tissue parenchyma coupled with the extrusion of S1P into plasma. About ⅓ of plasma S1P is provided by vascular endothelial cells via SPNS2⁴, with the remainder being released from red blood cells (RBCs) by a different S1P transporter. The transporter was subsequently discovered to be MFSD2B, which is an erythroid lineage-specific major facilitator superfamily member that is distantly related to SPNS2⁵′6. Germ line deletion of Mfsd2b results in a 50% decrease in plasma S1P but an astonishing 60-fold increase in RBC S1P; however, these animals are not lymphopenic⁵. RBCs lack S1P catabolic enzymes but express sphingosine kinase type 1 (SphK1), which accounts for the high levels of S1P in whole blood. Blood S1P gradients are necessary to maintain endothelial barrier integrity⁷. Indeed, Ma has proposed that vascular S1P gradients are a fundamental property of the closed circulatory systems of vertebrates⁹.

The role of the catabolic enzyme S1P lyase in maintaining low LN S1P predicts that S1P lyase inhibitors will eliminate the gradient, which will modulate the immune system by disrupting lymphocyte trafficking analogous to S1P1 agonists. Indeed, S1P lyase deficiency, whether accomplished through genetic manipulation of mice or S1P lyase inhibitor administration, raises S1P levels in tissues, including lymph nodes, with a resulting lymphopenia^{3,10}. However, administering a selective S1P lyase inhibitor to rats and inducing global deletion of the gene (Sgpl1) in mice were both found to be nephrotoxic¹⁰. Furthermore, humans deficient in S1P lyase activity because of SGPL1 variant alleles exhibit multiple pathologies including steroid resistant nephrosis, adrenal insufficiency, and ichthyosis^{11,12}. Such observations appear to eliminate S1P lyase as a therapeutic target.

Mice rendered deficient in Spns2 either through germ line or endothelium-specific deletion of Spns2, have a 10-fold decrease in S1P levels in thoracic duct lymph and are lymphopenic but the vascular S1P gradient is less affected (30% reduction in plasma S1P)⁴. These results validate the data disclosed herein that SPNS2 inhibitors of this disclosure, by preventing the formation of the lymph S1P gradient, recapitulate the therapeutic efficacy of S1P1 receptor agonists without their adverse events.

Application of an SPNS2 inhibitor in immuno-oncology comes from another mouse genetics study. In a screen of 810 mouse strains with different germ line gene deletions, Spns2⁴ mice were found to have remarkably low metastatic colonization of the lungs when injected with B16-F10 melanoma cells¹³. This effect was observed with other lung metastatic colonization models and in similar models in liver. As expected, the total number of immune cells in the lung was reduced in the lymphopenic Spns2^{-/-} mice, but the lung resident population was proportionally enriched in natural killer and CD8⁺ effector cells¹³.

Thus, results from the study of mice rendered deficient in Spns2 indicate that SPNS2 inhibitors are immunomodulatory. The SPNS2 inhibitors of the disclosure recapitulate the SPNS2 null phenotype, and they and enable S1P transport inhibition as a viable therapeutic strategy as well as providing heretofore unavailable chemical biology tools to explore S1P physiology in vivo.

### Definitions

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂ C(CH₃)₃, —CH₂C(CH₂CH₃)₃, —CH(CH₃)CH(CH₃)(CH₂CH₃), —CH₂CH₂CH(CH₃)₂, —CH₂CH₂C H(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and the like. Thus, alkyl groups include primary alkyl groups, secondary alkyl groups, and tertiary alkyl groups. An alkyl group can be unsubstituted or optionally substituted with one or more substituents as described herein below.

The phrase “substituted alkyl” refers to alkyl substituted at one or more positions, for example, 1, 2, 3, 4, 5, or even 6 positions, which substituents are attached at any available atom to produce a stable compound, with substitution as described herein. “Optionally substituted alkyl” refers to alkyl or substituted alkyl.

A “haloalkyl” is an alkyl, as defined herein, that is substituted with at least one, such as 1-8, halo substituents.

Each of the terms “halogen,” “halide,” and “halo” refers to —F, —Cl, —Br, or —I.

The term “alkenyl” refers to straight or branched chain hydrocarbyl groups including from 2 to about 20 carbon atoms having 1-3, 1-2, or at least one carbon to carbon double bond. An alkenyl group can be unsubstituted or optionally substituted with one or more substituents as described herein below.

“Substituted alkenyl” refers to alkenyl substituted at 1 or more, e.g., 1, 2, 3, 4, 5, or even 6 positions, which substituents are attached at any available atom to produce a stable compound, with substitution as described herein. “Optionally substituted alkenyl” refers to alkenyl or substituted alkenyl.

“Alkyne or “alkynyl” refers to a straight or branched chain unsaturated hydrocarbon having the indicated number of carbon atoms and at least one triple bond. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein below.

“Substituted alkynyl” refers to an alkynyl substituted at 1 or more, e.g., 1, 2, 3, 4, 5, or even 6 positions, which substituents are attached at any available atom to produce a stable compound, with substitution as described herein. “Optionally substituted alkynyl” refers to alkynyl or substituted alkynyl.

The term “alkoxy” refers to an —O-alkyl group having the indicated number of carbon atoms. For example, a (C₁-C₆)alkoxy group includes —O-methyl, —O-ethyl, —O-propyl, —O— isopropyl, —O-butyl, —O-sec-butyl, —O-tert-butyl, —O-pentyl, —O-isopentyl, —O-neopentyl, —O— hexyl, —O-isohexyl, and —O-neohexyl.

A “haloalkoxy” is an alkoxy, as defined herein, that is substituted with at least one, such as 1-8, halo substituents.

The term “cycloalkyl” refers to a monocyclic, bicyclic, tricyclic, or polycyclic, 3- to 14-membered ring system, which is either saturated, such as “cycloalkyl,” or unsaturated, such as “cycloalkenyl.” The term “cycloalkenyl” refers specifically to cyclic alkenyl, such as C₃-C₆-cycloalkenyl. The cycloalkyl may be attached via any atom. Cycloalkyl, for instance, also contemplates fused rings wherein, for instance, a cycloalkyl is fused to an aryl or heteroaryl ring as defined herein. Representative examples of cycloalkyl include, but are not limited to cyclopropyl, cyclobutyl, cyclopentyl, cyclohexyl, cyclopropenyl, cyclobutenyl, cyclopentenyl, and cyclohexenyl. A cycloalkyl group can be unsubstituted or optionally substituted with one or more substituents as described herein.

“Substituted cycloalkyl” refers to cycloalkyl substituted at 1 or more, e.g., 1, 2, 3, 4, 5, or even 6 positions, which substituents are attached at any available atom to produce a stable compound, with substitution as described herein. “Optionally substituted cycloalkyl” refers to cycloalkyl or substituted cycloalkyl.

“Aryl” when used alone or as part of another term means a carbocyclic aromatic group whether or not fused having the number of carbon atoms designated or if no number is designated, up to 14 carbon atoms, such as a C₆-C₁₄-aryl. Particular aryl groups are phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). A particular aryl is phenyl. “Aryl” also includes aromatic ring systems that are optionally fused with a cycloalkyl ring, as herein defined. An aryl group can be unsubstituted or optionally substituted with one or more substituents as described herein below.

A “substituted aryl” is an aryl that is independently substituted with one or more substituents attached at any available atom to produce a stable compound, wherein the substituents are as described herein. “Optionally substituted aryl” refers to aryl or substituted aryl.

The term “heteroatom” refers to N, O, and S. Disclosed compounds that contain N or S atoms can be optionally oxidized to the corresponding N-oxide, sulfoxide, or sulfone compounds.

“Heteroaryl,” alone or in combination with any other moiety described herein, refers to a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein below.

A “substituted heteroaryl” is a heteroaryl that is independently substituted, unless indicated otherwise, with one or more, e.g., 1, 2, 3, 4 or 5, also 1, 2, or 3 substituents, also 1 substituent, attached at any available atom to produce a stable compound, wherein the substituents are as described herein. “Optionally substituted heteroaryl” refers to heteroaryl or substituted heteroaryl.

“Heterocycloalkyl” means a saturated or unsaturated non-aromatic monocyclic, bicyclic, tricyclic or polycyclic ring system that has from 3 to 14, such as 3 to 6, atoms in which from 1 to 3 carbon atoms in the ring are replaced by heteroatoms of O, S or N. A heterocycloalkyl is optionally fused with aryl or heteroaryl of 5-6 ring members, and includes oxidized S or N, such as sulfinyl, sulfonyl and N-oxide of a tertiary ring nitrogen. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl. A hetercycloalkyl group can be unsubstituted or optionally substituted with one or more substituents as described herein below.

“Optionally substituted heterocycloalkyl” denotes a heterocycloalkyl that is substituted with 1 to 3 substituents, e.g., 1, 2 or 3 substituents, attached at any available atom to produce a stable compound, wherein the substituents are as described herein.

The term “nitrile” or “cyano” can be used interchangeably and refer to a —CN group which is bound to a carbon atom of a heteroaryl ring, aryl ring and a heterocycloalkyl ring.

The term “oxo” refers to a ═O atom attached to a saturated or unsaturated moiety. The ═O atom can be attached to a carbon, sulfur, or nitrogen atom that is part of a cyclic or acyclic moiety.

A “hydroxyl” or “hydroxy” refers to an —OH group.

The substituent —CO₂H may be replaced with bioisosteric replacements such as:

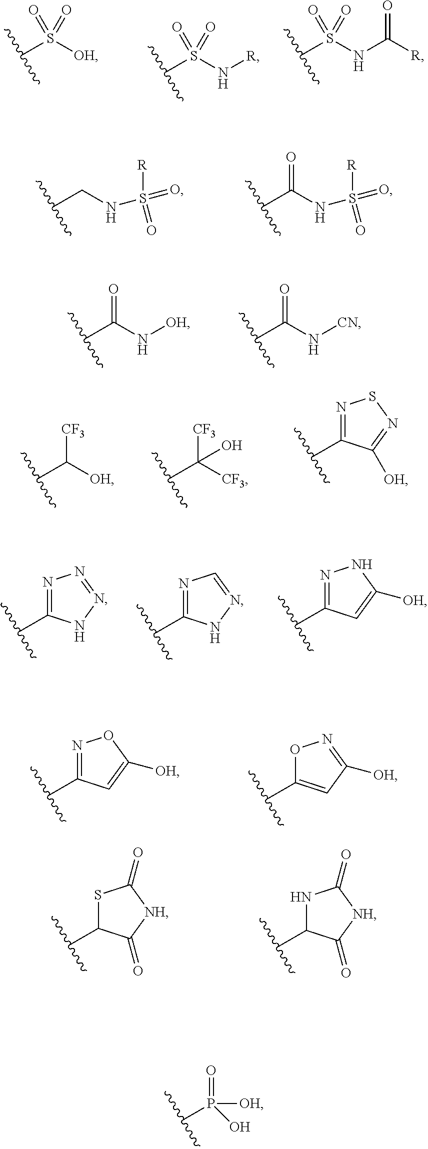

and the like, wherein R has the same definition as R^{A} as defined herein. See, e.g., THE PRACTICE OF MEDICINAL CHEMISTRY (Academic Press: New York, 1996), at page 203.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified to the contrary, the term “compound” is inclusive in that it encompasses a compound or a pharmaceutically acceptable salt, stereoisomer, and/or tautomer thereof. Thus, for instance, a compound of Formula I includes a pharmaceutically acceptable salt of the compound.

In this description, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2, 2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In certain embodiments, such terms refer to minimizing the spread or worsening of the disease resulting from the administration of one or more prophylactic or therapeutic agents to a patient with such a disease.

The terms “prevent,” “preventing,” and “prevention” refer to the prevention of the onset, recurrence, or spread of the disease in a patient resulting from the administration of a prophylactic or therapeutic agent.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or synergies with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult.

“Inhibitor” means a compound that induces dose dependent lymphopenia and a modest decrease in plasma S1P. In an embodiment, an inhibitor binds to SPNS2.

### Compounds

As described generally above, the present disclosure provides compounds, pharmaceutically acceptable salts, and/or tautomers thereof, wherein the compounds conform to Formula I:

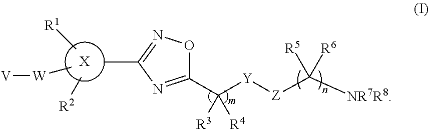

X is a C₆-C₁₀-aryl or 5- to 10-membered heteroaryl (wherein 1 to 4 heteroaryl ring members are independently selected from N, O, and S).

R¹ and R² are independently selected from the group consisting of H, C₁-C₆₋alkyl, C₁-C₆₋alkoxy, C₁₋₆₋haloalkoxy, C₃-C₈-cycloalkyl, C₁-C₆-haloalkyl, CN, halo, and —C(O)N(H)(C₁-C₆₋alkyl).

W is a bond, O, NH, —NHC(O)—, or —O—(N═)C(R)— (wherein R is H or C₁-C₆-alkyl).

V is selected from the group consisting of H, C₁-C₁₄-alkyl, C₂-C₁₂-alkenyl, (C₆-C₁₀)aryl, (C₆-C₁₀)heteroaryl, —C₁-C₁₀-alkyl-(C₆-C₁₀)aryl, —C₂-C₁₂-alkenyl-(C₆-C₁₀)aryl, —C₁-C₁₀-alkyl-(C₃-C₈)cycloalkyl, -(3- to 14-membered heterocycloalkyl) (wherein 1-4 heterocycloalkyl members are independently selected from N, O, and S), —(C₁-C₁₀)alkyl-(3- to 14-membered heterocycloalkyl) (wherein 1-4 heterocycloalkyl members are independently selected from N, O, and S).

Y is selected from a bond, —NH—, (C₆-C₁₀)arylenyl, and (C₃-C₈)cycloalkylenyl.

Z is selected from a bond and —C(O)—.

R³ and R⁴ are independently selected from the group consisting of, H, C₁-C₆-alkyl, OH, C₁-C₆₋alkoxy, halo, —NRR′, —C(O)R, and —C(O)OR, wherein R and R′ are independently selected from H and C₁-C₆-alkyl.

R⁵ and R⁶ are independently selected from the group consisting of, H, C₁-C₆-alkyl, OH, C₁-C₆₋alkoxy, halo, —C(O)R, and —C(O)OR, wherein R is H or C₁-C₆-alkyl, or R⁵ and R⁶, together with the carbon to which they are bound, form a —(C₃-C₈)cycloalkyl.

Subscript m is an integer selected from 0, 1, 2, 3, 4, 5, and 6.

Subscript n is an integer selected from 0, 1, and 2.

R⁷ and R⁸ are independently selected from the group consisting of H and C₁-C₆-alkyl, or R⁷ and R⁸, together with the nitrogen atom to which they are bound, form a 5- to 7-membered heterocycloalkyl (wherein 1-4 other heterocycloalkyl members are optionally independently selected from NH, O, and S).

Optionally, in some embodiments, one of R⁵ and R⁶ and one of R⁷ and R⁸, together with the carbon and nitrogen atoms to which they are bound, respectively, form a 4- to 7-membered heterocycloalkyl (wherein 1-4 other heterocycloalkyl members are optionally independently selected from NH, O, and S). In additional embodiments, the heterocycloalkyl is a 5- to 7-membered heterocycloalkyl.

Each alkyl, alkoxy, alkenyl, alkynyl, aryl, cycloalkyl, heterocycloalkyl, and heteroaryl is optionally substituted with 1-5 substituents independently selected from the group consisting of hydroxy, halo, C₁-C₆₋haloalkoxy, C₁-C₆-haloalkyl, —NR″₂, —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂—(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl).

Each R″ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S.

In some embodiments, X is phenyl.

In various embodiments, optionally in combination with any other embodiment herein described, each of R¹ and R² is H.

In still further embodiments, optionally in combination with any other embodiment herein described, each of Y and Z is a bond.

In some embodiments, optionally in combination with any other embodiment herein described, m is selected from 0, 1, 2, 3, and 4. For example, m is 1, 2, or 3. In illustrative embodiments, m is 3 and n is 1 or 2.

In other embodiments, optionally in combination with any other embodiment herein described, R⁷ and R⁸ are independently selected from the group consisting of H and C₁-C₆-alkyl. For example, in some embodiments, each of R⁷ and R⁸ is H.

In various embodiments, optionally in combination with any other embodiment herein described, Y is (C₃-C₈)cycloalkylenyl and Z is a bond. Various combinations are contemplated in this context: in an embodiment, m is 0 or 1. In another embodiment, m is 0. In still another embodiment, optionally in combination with other embodiments described herein, n is 0.

In additional embodiments, optionally in combination with any other embodiment herein described, Y is —NH—. Further, optionally in combination with this embodiment, Z is —C(O)—. Variations of these embodiments reside in the provision of m as 0 or 1.

In still further embodiments, optionally in combination with any other embodiment herein described, one of R⁵ and R⁶ and one of R⁷ and R⁸, together with the carbon and nitrogen atoms to which they are bound, respectively, form a 5- to 7-membered heterocycloalkyl. In an embodiment, the other one of R⁷ and R⁸ is H.

The present disclosure provides specific examples of Formula I compounds, and their pharmaceutically acceptable salts, and/or tautomers thereof as set forth in Table 1 below.

TABLE 1
Examples of Formula I Compounds

| Compound | Structure |
|---|---|
| 5a | 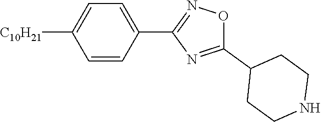 |
| 5b | 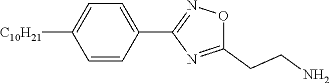 |
| 5c | 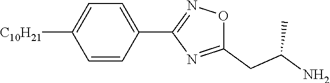 |
| 5d | 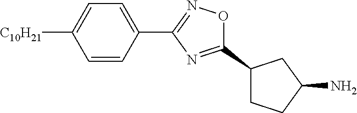 |
| 5e | 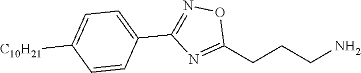 |
| 5f | 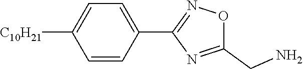 |
| 5g | 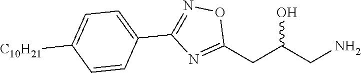 |
| 5h | 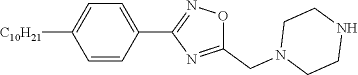 |
| 5i | 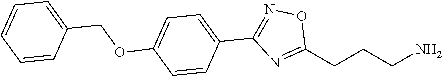 [SMILES: NCCCc1nc(-c2ccc(OCc3ccccc3)cc2)no1] |
| 5j | 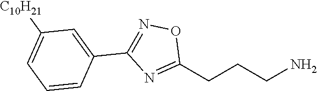 |
| 5k | 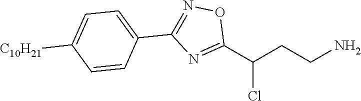 |
| 5l | 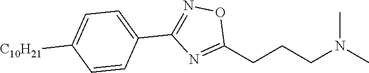 |
| 5m | 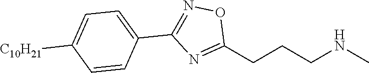 |
| 5n | 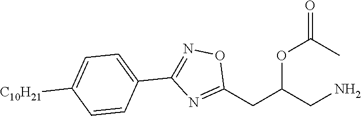 |
| 5n | 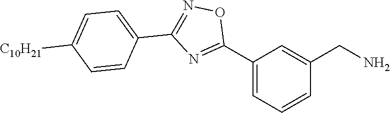 |
| 5p | 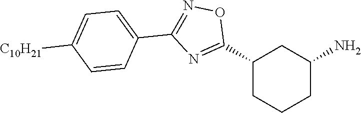 |
| 5q | 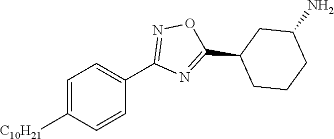 |
| 5r | 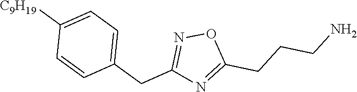 |
| 5s | 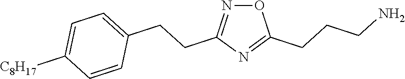 |
| 5t | 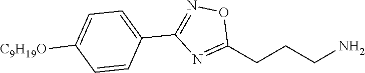 |
| 5u | 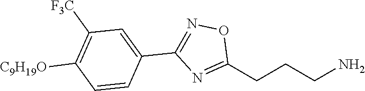 |
| 5v | 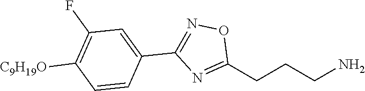 |
| 5w | 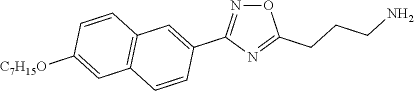 |
| 5x | 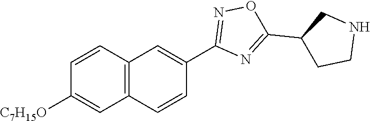 |
| 5y | 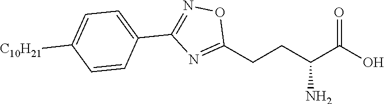 |
| 6a | 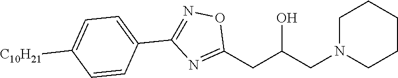 |
| 6b | 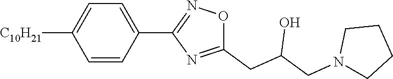 |
| 6c | 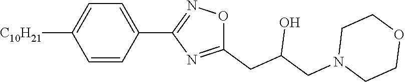 |
| 6d | 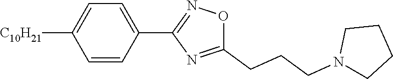 |
| 6e | 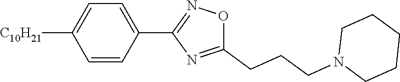 |
| 6f | 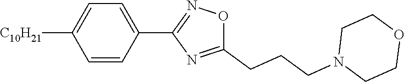 |
| 7a | 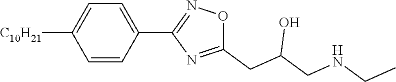 |
| 7b | 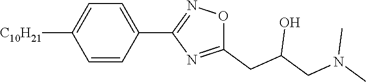 |
| 9a | 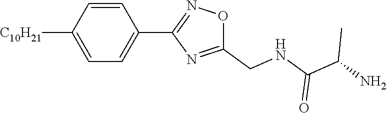 |
| 9b | 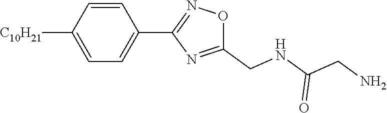 |
| 9c | 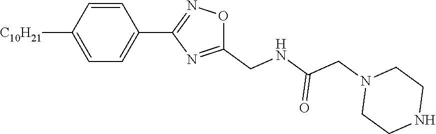 |
| 9d | 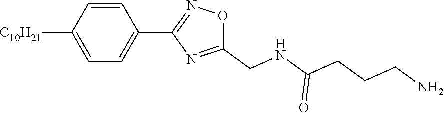 |
| 9e | 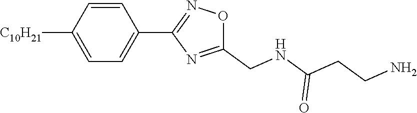 |
| 9f | 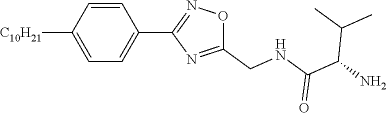 |
| 9g | 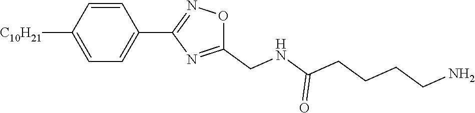 |
| 9h | 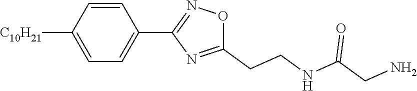 |
| 9i | 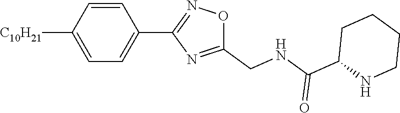 |
| 9j | 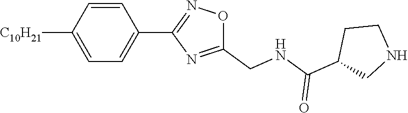 |
| 9k | 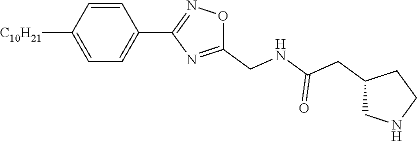 |
| 9l | 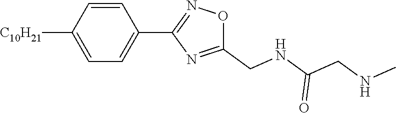 |
| 9m | 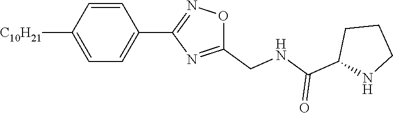 |
| 9n | 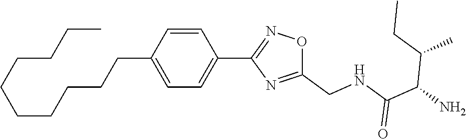 [SMILES: CCCCCCCCCCc1ccc(-c2noc(CNC(=O)[C@@H](N)[C@@H](C)CC)n2)cc1] |
| 9o | 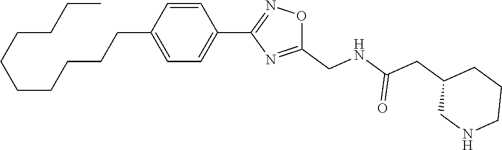 [SMILES: CCCCCCCCCCc1ccc(-c2noc(CNC(=O)C[C@@H]3CCCNC3)n2)cc1] |
| 9p | 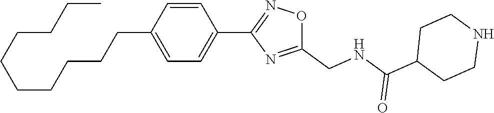 [SMILES: CCCCCCCCCCc1ccc(-c2noc(CNC(=O)C3CCNCC3)n2)cc1] |
| 9q | 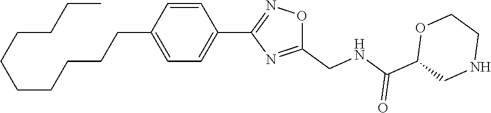 [SMILES: CCCCCCCCCCc1ccc(-c2noc(CNC(=O)[C@H]3CNCCO3)n2)cc1] |
| 9r | 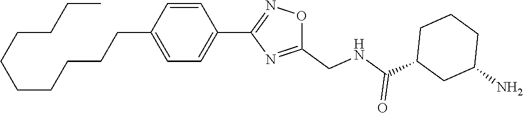 [SMILES: CCCCCCCCCCc1ccc(-c2noc(CNC(=O)[C@@H]3CCC[C@H](N)C3)n2)cc1] |
| 9s | 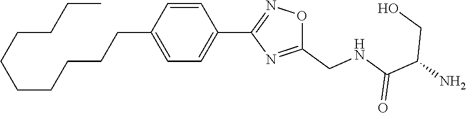 [SMILES: CCCCCCCCCCc1ccc(-c2noc(CNC(=O)[C@@H](N)CO)n2)cc1] |
| 9t | 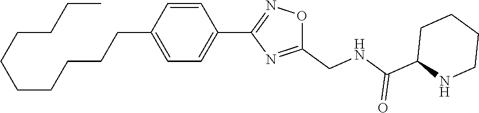 [SMILES: CCCCCCCCCCc1ccc(-c2noc(CNC(=O)[C@H]3CCCCN3)n2)cc1] |
| 9u | 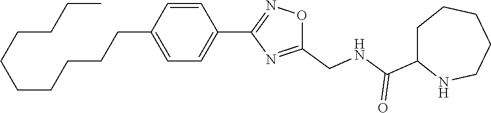 [SMILES: CCCCCCCCCCc1ccc(-c2noc(CNC(=O)C3CCCCCN3)n2)cc1] |
| 9v | 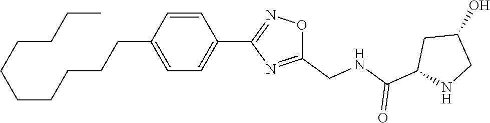 [SMILES: CCCCCCCCCCc1ccc(-c2noc(CNC(=O)[C@@H]3C[C@H](O)CN3)n2)cc1] |
| 9w | 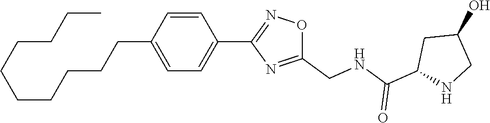 [SMILES: CCCCCCCCCCc1ccc(-c2noc(CNC(=O)[C@@H]3C[C@@H](O)CN3)n2)cc1] |
| 9x | 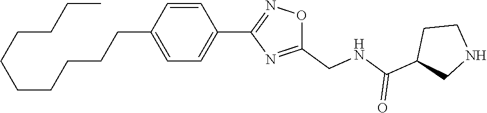 [SMILES: CCCCCCCCCCc1ccc(-c2noc(CNC(=O)[C@H]3CCNC3)n2)cc1] |
| 9y | 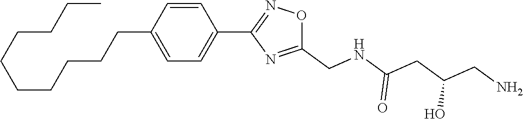 [SMILES: CCCCCCCCCCc1ccc(-c2noc(CNC(=O)C[C@@H](O)CN)n2)cc1] |
| 9z | 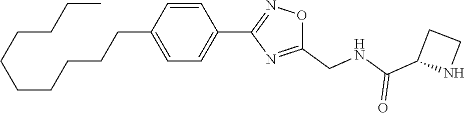 [SMILES: CCCCCCCCCCc1ccc(-c2noc(CNC(=O)[C@@H]3CCN3)n2)cc1] |
| 9aa | 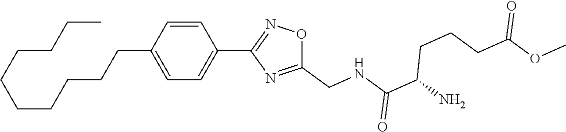 [SMILES: CCCCCCCCCCc1ccc(-c2noc(CNC(=O)[C@@H](N)CCCC(=O)OC)n2)cc1] |
| 9ab | 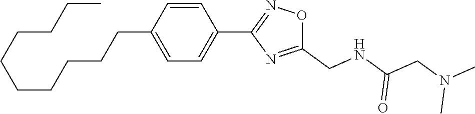 [SMILES: CCCCCCCCCCc1ccc(-c2noc(CNC(=O)CN(C)C)n2)cc1] |
| 9ac | 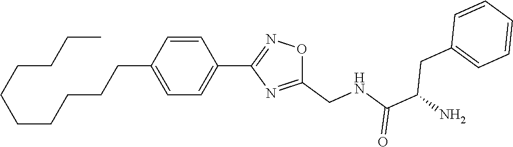 [SMILES: CCCCCCCCCCc1ccc(-c2noc(CNC(=O)[C@@H](N)Cc3ccccc3)n2)cc1] |
| 9ad | 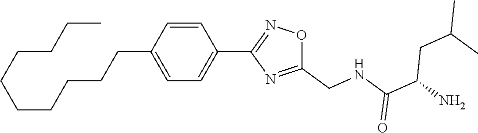 [SMILES: CCCCCCCCCCc1ccc(-c2noc(CNC(=O)[C@@H](N)CC(C)C)n2)cc1] |
| 9ae | 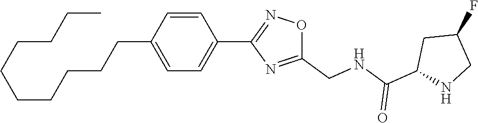 [SMILES: CCCCCCCCCCc1ccc(-c2noc(CNC(=O)[C@@H]3C[C@@H](F)CN3)n2)cc1] |
| 9af | 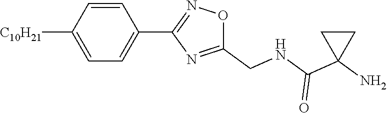 |
| 9ag | 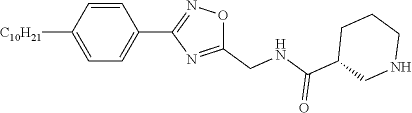 |
| 9ah | 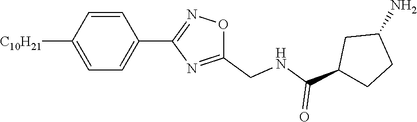 |
| 9ai | 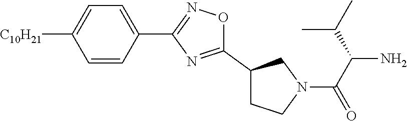 |
| 9aj | 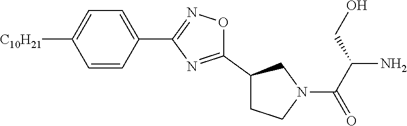 |
| 9ak | 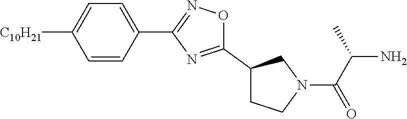 |
| 9al | 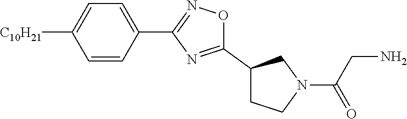 |
| 13a | 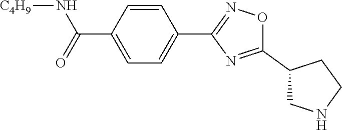 |
| 13b | 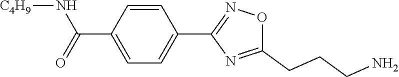 |
| 13c | 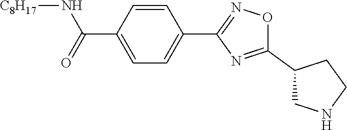 |
| 13d | 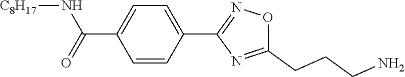 |
| 16a | 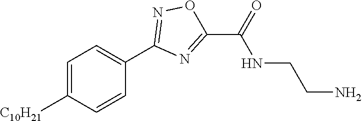 |
| 16b | 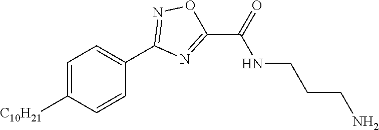 |
| 19a | 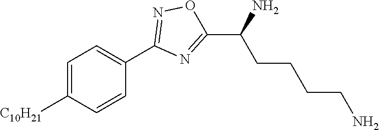 |
| 19b | 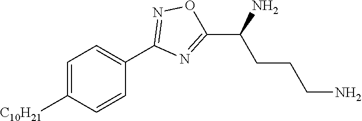 |
| 19c | 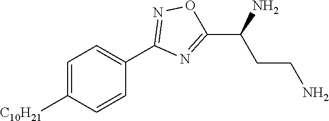 |
| 19d | 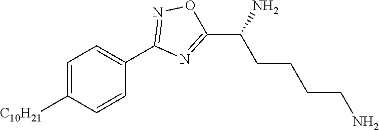 |
| 21 | 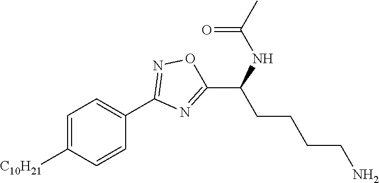 |
| 28 | 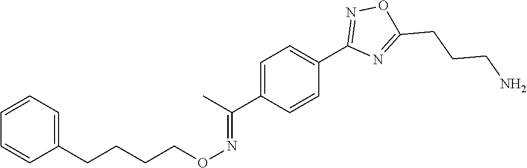 [SMILES: C/C(=N\OCCCCc1ccccc1)c1ccc(-c2noc(CCCN)n2)cc1] |

Pharmaceutical Composition

The disclosure also provides a pharmaceutical composition comprising a therapeutically effective amount of one or more compounds according to Formula I or a pharmaceutically acceptable salt, stereoisomer, and/or tautomer thereof in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Table 1 or a pharmaceutically acceptable salt, stereoisomer, and/or tautomer thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound (or a pharmaceutically acceptable salt, stereoisomer, and/or tautomer thereof that is administered is governed by such considerations, and is the minimum amount necessary to induce dose dependent lymphopenia and a modest decrease in plasma S1P, or to inhibit SPNS2 activity, or both. Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 1 mg to about 1000 mg of a compound (or a pharmaceutically acceptable salt, stereoisomer, or tautomer thereof) of the present disclosure. In another embodiment, such dosage forms contain from about 50 mg to about 500 mg of a compound (or a pharmaceutically acceptable salt, stereoisomer, or tautomer thereof) of the present disclosure. In yet another embodiment, such dosage forms contain from about 25 mg to about 200 mg of a compound (or a pharmaceutically acceptable salt, stereoisomer, or tautomer thereof) of the present disclosure. In still another embodiment, such dosage forms contain from about 10 mg to about 100 mg of a compound (or a pharmaceutically acceptable salt, stereoisomer, or tautomer thereof) of the present disclosure. In a further embodiment such dosage forms contain from about 5 mg to about 50 mg of a compound (or a pharmaceutically acceptable salt, stereoisomer, or tautomer thereof) of the present disclosure.

The disclosed compositions can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups, or elixirs.

Also encompassed by the present disclosure are pharmaceutical compositions suitable for single unit dosages that comprise a compound of the disclosure or its pharmaceutically acceptable stereoisomer, salt, or tautomer and a pharmaceutically acceptable carrier.

Compositions suitable for oral use may be prepared according to any method known to the art for the manufacture of pharmaceutical compositions. For instance, liquid formulations of the inventive compounds contain one or more agents selected from the group consisting of sweetening agents, flavoring agents, coloring agents and preserving agents in order to provide pharmaceutically palatable preparations of the SPNS2 inhibitor.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### Methods of Use

S1P gradients are chemotactic, a property that enables correct positioning of immune cells, and they help to maintain endothelial barrier integrity. Accordingly, S1P gradients are manipulated for therapeutic benefit using Formula I compounds because they target the endothelial S1P exporter, SPNS2.

Thus, in one embodiment, the disclosure provides a method of inhibiting spinster homolog 2 (SPNS2). The method comprises contacting SPNS2 with an effective amount of a compound as described herein. In some embodiments, the contacting occurs in vitro. In other embodiments, the contacting occurs ex vivo or in vivo.

Another embodiment is a method of treating a patient afflicted by a neoplastic disease, comprising administering to the patient a therapeutically effective amount a compound as described herein. In some embodiments, the neoplastic disease is metastatic neoplasms.

An additional embodiment is a method of treating a patient afflicted with an allergic disease, comprising administering to the patient a therapeutically effective amount of a compound as described herein. An illustrative allergic disease is asthma.

Formula I compounds also are useful in a method of treating a patient afflicted with an autoimmune disease, comprising administering to the patient a therapeutically effective amount of the compound. In various embodiments, the autoimmune disease is chosen from multiple sclerosis, type I diabetes, inflammatory bowel diseases, Crohn's disease, ulcerative colitis, Grave's disease, Addison's disease, dermatomyositis, myasthenia gravis, systemic lupus erythematosus, scleroderma, and psoriasis. An exemplary autoimmune disease is multiple sclerosis. In accordance with some embodiments, multiple sclerosis comprises one or more progressive forms of multiple sclerosis as well as the remitting relapsing form of the disease.

Additional embodiments include a method of treating a patient afflicted with atherosclerosis or pulmonary arterial hypertension. The method comprises administering to the patient a therapeutically effective amount of a compound as described herein.

### LITERATURE CITED IN THE PRESENT DISCLOSURE

1. Chun J, Brinkmann V. A mechanistically novel, first oral therapy for multiple sclerosis: the development of fingolimod (FTY720, Gilenya). Discovery Medicine 12, 213-228 (2011) PMC3694367.
2. Cusack K P, Stoffel R H. SP(1) receptor agonists: Assessment of selectivity and current clinical activity. Current Opinion Drug Discovery & Development 13, 481-488 (2010).
3. Schwab S R, Pereira J P, Matloubian M, Y. Xu Y, Huang Y, Cyster J G. Lymphocyte sequestration through S1P lyase inhibition and disruption of S1P gradients. Science 309, 1735-1739 (2005).
4. Mendoza A, Breat B, Ramoz-Perez W D, Pitt L A, Gobert M, Sunkara M, Lafaille J J, Morris A J, Schwab S R. The transporter SPNS2 is required for secretion of lymph but not plasma sphingosine-1-phosphate. Cell Reports 2, 1104-1110 (2012) PMC3616496.
5. Vu T M, Ishizu A-N, Foo J C, Toh X R, Zhang F, Whee D M, Torta F, Cazenave-Gassiot A, Matsumura T, Kim S, To S-AES, Suda T, Silver D L, Wenk M R, Nguyen L N. MFSD2B is essential for the sphingosine-1-phosphate export in erythrocytes and platelets. Nature 550, 524-528 (2017).
6. Kobayashi N, Kawasaki-Nishi S, Otsuka M, Hisano Y, Yamaguchi A, Nishi T. MSFD2B is a sphingosine 1-phosphate transporter in erythroid cells. Scientific Reports 8, 4969 (2018).
7. Camerer E, Regard J B, Cornelisssen I, Srinivasan Y, Duong D N, Palmer D, Pham T H, Wong J S, Pappu R, Coughlin S R. Sphingosine-1-phosphate in the plasma compartment regulates basal and inflammation-induced vascular leak in mice. J Clinical Investigation 119, 1871-1879 (2009).
8. Xiong Y, Hia T. S1P control of endothelial integrity. Current Topics in Microbiology & Immunology 378, 85-105 (2014) PMC4240614.
9. Yanagida K, Hla T. Vascular and immunobiology of the circulatory sphingosine 1-phosphate gradient. Annual Review Physiology 79, 67-91 (2016) PMC5500220.
10. Schumann J, Grevot A, Ledieu D, Wolf A, Schubart A, Piaia A, Sutter E, Cate S, Beerli C, Pognan F, Billich A, Moulin P, Walker U J. Reduced activity of sphingosine-1-phosphate lyase induces podocyte-related glomerular proteinuria, skin irritation, and platelet activation. Toxicologic Pathology 43, 694-703 (2015).
11. Prasad R, Hadjidemetriou I, Maharaj A, Meimaridou E, Buonocore F, Saleem M, Hurcombe J, Bierzynska A, Barbagelata E, Bergadá I, Cassinelli H, Das U, Krone R, Hacihamdioglu B, Sari E, Yesilkaya E, Storr H L, Clemente M, Fernandez-Cancio M, Camats N, Ram N, Achermann J C, Van Veldhoven P P, Guasti L, Braslavsky D, Guran T, Metherell L A. Sphingosine 1-phosphate lyase mutations cause primary adrenal insufficiency and steroid-resistant nephrotic syndrome. J Clinical Investigation 127, 942-953 (2017) PMC5330744.
12. Lovric S, Goncalves S, Gee H Y, Oskouian B, Srinivas H, Choi W I, Shril S, Ashraf S, Tan W, Rao J, Airik M, Schapiro D, Braun D A, Sadowski C E, Widmeier E, Jobst-Schwan T, Schmidt J M, Girik V, Capitani G, Suh J H, Lachaussée N, Arrondel C, Patat J, Gribouval O, Furlano M, Boyer O, Schmitt A, Vuiblet V, Hashmi S, Wilcken R, Bernier F P, Innes A M, Parboosingh J S, Lamont R E, Midgley J P, Wright N, Majewski J, Zenker M, Schaefer F, Kuss N, Greil J, Giese T, Schwarz K, Catheline V, Schanze D, Franke I, Sznajer Y, Truant A S, Adams B, Desir J, Biemann R, Pei Y, Ars E, Lloberas N, Madrid A, Dharnidharka V R, Connolly A M, Willing M C, Cooper M A, Lifton R P, Simons M, Riezman H, Antignac C, Saba J D, Hildebrandt F. Mutations in sphingosine-1-phosphate lyase cause nephrosis with ichthyosis and adrenal insufficiency. J Clinical Investigation 127, 912-928 (2017) PMC5330730.
13. van der Weyden L, Arends M J, Campbell A D, Bald T, Wardle-Jones H, Griggs N, Velasco-Herrera M D, Tuting T, Sansom O J, Karp N A, Clare S, Gleeson D, Ryder E, Galli A, Tuck E, Cambridge E L, Voet T, Macaulay I C, Wong K, Sanger Mouse Genetics Project, Spiegel S, Speak A O, Adams D J. Genome-wide in vivo screen identifies novel new regulators for metastatic colonization. Nature 541, 233-236 (2017) PMC5603286.

### EXAMPLES

The present disclosure will be more fully understood by reference to the following examples. The examples should not, however, be construed as limiting the scope of the present disclosure.

General Procedure 1: Suzuki-Miyaura Cross Coupling

To a round bottom flask a containing alkene (1.1 equiv) in THF was added 9BBN (1.5 equiv) and then heated to reflux until consumption of alkene as monitored by TLC (30-60 minutes). Aryl iodide (1 equiv) and Pd(dppf)Cl₂ (0.05 equiv) were then added to the mixture, followed by dropwise addition of a 3M KOH(.q) solution (3 equiv). The resulting mixture was then heated to reflux until consumption of aryl iodide as monitored by TLC (2-6 hours). Upon cooling to room temperature, the reaction mixture was filtered over a pad of celite, diluted in ethyl acetate, and washed with a brine solution. The organic layer was then dried over sodium sulfate and concentrated in vacuo to afford the crude product as a yellow oil, which was then purified by column chromatography with an appropriate hexanes:ethyl acetate solvent system to afford the pure product.

General Procedure 2: Amidoxime Synthesis A

To a round bottom flask containing ethanol was added 4-decylbenzonitrile (1 equiv), hydroxylamine hydrochloride (2 equiv), and triethylamine (3 equiv) under ambient air. The reaction mixture was then heated to reflux until complete as monitored by TLC (1-4 hours). The resulting solution was allowed to cool to room temperature, followed by concentration in vacuo, to afford the crude mixture as a solid. Purification by column chromatography (0-20% ethyl acetate in dichloromethane) afforded the pure amidoxime product.

General Procedure 3: Amidoxime Synthesis B

To a round bottom flask containing ethanol:water (1:1) was added benzonitrile (1 equiv), hydroxylamine hydrochloride (2 equiv), and sodium carbonate (5 equiv) under ambient air. The reaction mixture was then heated to reflux until complete as monitored by TLC (1-6 hours). The resulting solution was allowed to cool to room temperature. A white precipitate formed upon cooling which was vacuum filtered over a filter frit and washed with water and ethanol. This afforded the pure amidoxime product.

General Procedure 4: 1,2,4-Oxadiazole Synthesis

Amidoxime (1 equiv), N-Boc protected amino acid (1.1 equiv), and DIEA (1.8 equiv) were added to a round bottom flask containing DMF at room temperature. HCTU (1.1 equiv) was then added and the resulting mixture was heated to 100° C. until completion as monitored by TLC (6-16 hours). Upon cooling to room temperature, the resulting mixture was diluted in ethyl acetate and washed with a saturated lithium bromide solution. The resulting aqueous layer was then extracted with ethyl acetate. The organic layers were then combined and washed with a brine solution, followed by drying over anhydrous sodium sulfate. Concentration in vacuo afforded the crude product, which was then purified by column chromatography using the appropriate ethyl acetate:hexanes solvent system to afford the pure 1,2,4-oxadiazole product.

General Procedure 5: TFA Boc Deprotection

To a round bottom flask containing Boc-protected amine or diboc protected guanidine compounds (1 equiv) dissolved in dichloromethane was added TFA (30 equiv). The resulting solution was allowed to stir until consumption of starting material as monitored by TLC (1-6 hours). Concentration in vacuo and filtration of the resulting off-white solid, followed by washing with diethyl ether afforded the corresponding TFA salts.

General Procedure 6: HCl Boc Deprotection A

To a 6-dram vial containing boc protected amine (1 equiv) was added hydrogen chloride (30-100 equiv, 4M in dioxane). The resulting mixture was allowed to stir until consumption of starting material as monitored by TLC (0.5-6 hours). A thick white precipitate forms during the course of the reaction. This precipitate was vacuum filtered over a filter frit and washed with diethyl ether to afford the pure product as an HCl salt.

General Procedure 7: Microwave Assisted Cyclization of Primary Amines

To a microwave vial were added primary amine salt (1 equiv), water, K₂CO₃ (1.1 equiv), and dibromoalkane at rt. The vial was then heated to 120° C. for 20 minutes in a CEM Discover SP Microwave Synthesizer. The reaction was then extracted with ethyl acetate and washed with a 50:50 brine:10% NaOH solution. The organic layer was then dried over anhydrous sodium sulfate and concentrated in vacuo to afford the crude product as a colorless oil. The crude product was then subjected to flash chromatography in an appropriate methanol:dichloromethane solvent system. The chromatographed material was then dissolved in methanolic HCl and concentrated in vacuo to afford the cyclic amine HCl salt.

General Procedure 8: HCTU Coupling

To a 6-dram vial containing N-boc-amino acid (1.1 equiv) was added DMF (0.2 M), DIEA (1.8 equiv) and HCTU (1.1 equiv). The resulting mixture was allowed to stir at rt for 5 minutes, followed by addition of aniline derivative (1 equiv). The resulting mixture was allowed to stir at rt until consumption of aniline as monitored by TLC (1-4 hours). The resulting reaction mixture was diluted in ethyl acetate and washed with a saturated lithium bromide solution. The organic layer was then dried over anhydrous sodium sulfate and concentrated in vacuo to afford an orange oil which was then subjected to flash chromatography with an appropriate ethyl acetate in dichloromethane solvent system to afford the pure product.

General Procedure 9: 1,2,4-Oxadiazole Synthesis

Amidoxime (1 equiv), acyl chloride or carboxylic acid (1.1 equiv), and DIEA (1.1 equiv) were added to a round bottom flask containing THF at room temperature. The resulting mixture was heated to 60° C. for 12 hours. Concentration in vacuo afforded the crude product, which was then purified by column chromatography using the appropriate ethyl acetate:hexanes solvent system to afford the pure 1,2,4-oxadiazole product.

General Procedure 10: Amide Synthesis

Ester (1 equiv) and primary amine (1.5 equiv) were added to a round bottom flask followed by 1:1 MeOH:DCM. The resulting reaction mixture was stirred at room temperature for 2 hours. Concentration in vacuo afforded the crude product, which was then purified by column chromatography using the appropriate ethyl acetate:hexanes solvent system to afford the pure product.

General Procedure 11: Removal of Fmoc Protecting Group

To a round bottom flask containing a stir bar and an Fmoc-protected amine was added morpholine (60 equiv) and dichloromethane. The reaction was allowed to stir overnight for 18 hours. Upon completion of the reaction, the mixture was concentrated under reduced pressure and subjected to silica gel chromatography with an appropriate ethyl acetate:hexanes solvent system to afford the pure product.

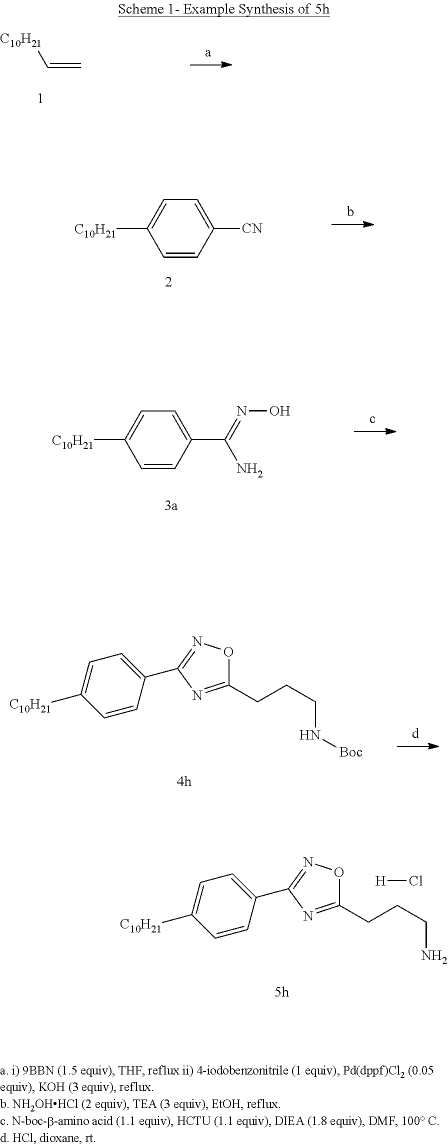

### N′-hydroxy-4-iodobenzimidamide (3a)

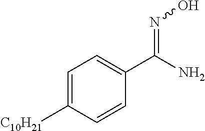

Synthesized according to General Procedure 2. Isolated as a mixture of Z:E (20:1). White solid, 560 mg (76%) yield. ¹H NMR (400 MHz, CDCl₃) δ 7.54 (d, J=8.2 Hz, 2H), 7.20 (d, J=8.2 Hz, 2H), 4.89 (brs, 2H), 2.62 (t, J=7.7 Hz, 2H), 1.66-1.55 (m, 2H), 1.37 (m, 14H), 0.88 (t, J=6.7 Hz, 3H).

### (Z)-2-(4-bromophenyl)-N′-hydroxyacetimidamide (3b)

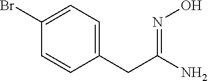 [SMILES: N/C(Cc1ccc(Br)cc1)=N\O]

Synthesized according to General Procedure 3. White solid (62%, 542 mg). ¹H NMR (400 MHz, CD₃OD) δ 7.44 (d, J=8.4 Hz, 2H), 7.23 (d, J=8.4 Hz, 2H), 3.34 (s, 2H). ¹³C NMR (101 MHz, CD₃OD) δ 155.5, 137.7, 132.5, 131.6, 121.5, 37.5. HRMS: (ESI) [M+H]+ calc. for C₈H₁₀BrN₂O, 228.9971, observed, 228.9977.

### (Z)-3-(4-bromophenyl)-N′-hydroxypropanimidamide (3c)

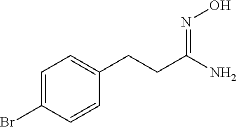 [SMILES: N/C(CCc1ccc(Br)cc1)=N\O]

Synthesized according to General Procedure 3. White solid (41%, 352 mg). ¹H NMR (400 MHz, CD₃OD) δ 7.40 (d, J=8.2 Hz, 2H), 7.15 (d, J=8.2 Hz, 2H), 2.84 (d, J=7.7 Hz, 2H), 2.34 (d, J=7.7 Hz, 2H). ¹³C NMR (101 MHz, CD₃OD) δ 156.4, 141.5, 132.4, 131.5, 120.8, 33.8, 33.7. HRMS: (ESI) [M+H]+ calc. for C₉H₁₂BrN₂O, 243.0128, observed, 243.0135.

### (Z)—N′-hydroxy-4-(nonyloxy)benzimidamide (3d)

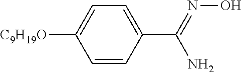

Synthesized according to General Procedure 3. White solid (35%, 320 mg). ¹H NMR (400 MHz, CDCl₃) δ 8.76 (s, 1H), 7.64 (d, J=8.9 Hz, 2H), 6.91 (d, J=8.8 Hz, 2H), 5.38 (s, 2H), 4.01 (t, J=6.5 Hz, 2H), 1.82-1.72 (m, 2H), 1.53-1.43 (m, 2H), 1.42-1.22 (m, 10H), 0.88 (t, J=6.8 Hz, 3H). ¹³C NMR (101 MHz, CDCl₃) δ 160.9, 152.0, 127.6, 126.8, 114.8, 68.6, 32.6, 30.3, 30.1, 30.0, 30.0, 26.8, 23.3, 14.4. HRMS: (ESI) [M+H]+ calc. for C₁₆H₂₇N₂O₂, 279.2073, observed, 279.2061.

### (Z)—N′-hydroxy-4-(nonyloxy)-3-(trifluoromethyl)benzimidamide (3e)

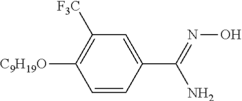

Synthesized according to General Procedure 3. White solid (89%, 616 mg). ¹H NMR (400 MHz, Acetone-d₆) δ 9.0 (s, 1H), 8.0 (d, J=2.3 Hz, 1H), 7.9 (dd, J=8.7, 2.0 Hz, 1H), 7.2 (d, J=8.7 Hz, 1H), 5.6 (s, 2H), 4.2 (t, J=6.3 Hz, 2H), 1.9-1.8 (m, 2H), 1.6-1.5 (m, 2H), 1.4-1.2 (m, 10H), 0.9 (t, J=6.8 Hz, 3H). ¹³C NMR (101 MHz, Acetone-d₆) δ 157.6 (d, J=1.6 Hz), 150.1, 130.7, 125.5, 124.0 (q, J=5.4 Hz), 123.9 (q, J=271.7 Hz), 117.7 (q, J=30.6 Hz), 112.9, 68.7, 31.7, 29.0, 28.8, 25.6, 22.4, 13.4. HRMS: (ESI) [M+H]+ calc. for C₁₇H₂₆N₂OF₃, 347.1946, observed, 347.1944.

### 6-(heptyloxy)-N′-hydroxy-2-naphthimidamide (3f)

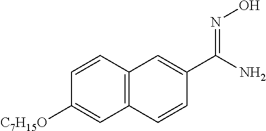

Synthesized according to General Procedure 3. Purified by silica plug (dichloromethane wash followed by elution with 100% ethyl acetate). Grey solid (93%, 2.1 g). ¹H NMR (400 MHz, acetone-d₆) δ 8.98 (s, 1H), 8.13 (d, J=1.7 Hz, 1H), 7.85 (dd, J=8.7, 1.8 Hz, 1H), 7.81 (d, J=8.9 Hz, 1H), 7.74 (d, J=8.7 Hz, 1H), 7.29 (d, J=8.7 Hz, 1H), 7.16 (dd, J=8.9, 2.5 Hz, 1H), 5.56 (s, 2H), 4.12 (t, J=6.5 Hz, 2H), 1.89-1.79 (m, 2H), 1.57-1.47 (m, 2H), 1.45-1.28 (m, 7H), 0.90 (t, J=6.8 Hz, 3H). ¹³C NMR (101 MHz, acetone-d₆) δ 158.6, 152.1, 136.1, 130.6, 129.6, 129.3, 127.3, 125.0, 124.8, 120.1, 107.4, 69.6, 32.6, 30.0, 29.8, 26.8, 23.3, 14.4. HRMS: (ESI) [M+H]+ calc. for C₁₈H₂₅N₂O₂, 301.1911, observed, 301.1912.

### tert-butyl 4-(3-(4-decylphenyl)-1,2,4-oxadiazol-5-yl)piperidine-1-carboxylate (4a)

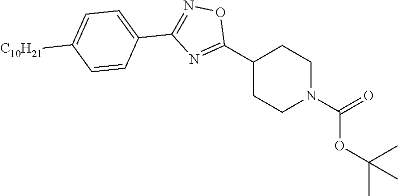

Synthesized by General Procedure 4. Purified by silica chromatography (14% EtOAc in hexanes). Yellow oil, 335 mg (76%) yield. ¹H NMR (400 MHz, CDCl₃) δ 7.97 (d, J=8.3 Hz, 2H), 7.27 (d, J=8.3 Hz, 2H), 4.11 (brs, 2H), 3.18-3.09 (m, 1H), 298 (t, J=11.6 Hz, 1H), 2.64 (t, J=7.7 Hz, 2H), 2.14-2.05 (m, 2H), 1.94-1.82 (m, 2H), 1.67-1.56 (m, 2H), 1.48 (brs, 9H), 1.37-1.19 (m, 14H), 0.87 (t, J=6.9 Hz, 3H). ¹³C NMR (100 MHz, CDCl₃) δ 181.0, 168.2, 154.5, 146.4, 128.8, 127.3, 124.2, 79.7, 42.8, 35.9, 34.4, 31.9, 31.2, 29.6, 29.6, 29.5, 29.3, 29.2, 29.1, 28.4, 22.7, 14.1. HRMS: (ESI) [M+H]+ calc. for C₂₈H₄₄N₃O₃, 470.3377, observed, 470.3369.

### tert-butyl (R)-(1-(3-(4-decylphenyl)-1,2,4-oxadiazol-5-yl)-3-methylbutan-2-yl)carbamate (4b)

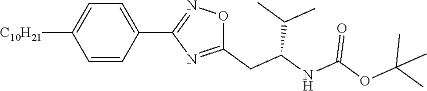

Synthesized according to General Procedure 4. Purified by silica chromatography (18% EtOAc in hexanes). Light yellow solid, 125 mg (37%) yield. ¹H NMR (400 MHz, CDCl₃) δ 7.97 (d, J=8.0 Hz, 2H), 7.27 (d, J=8.0 Hz, 2H), 4.91 (d, J=9.4 Hz, 1H), 3.97-3.87 (m, 1H), 3.17-3.06 (m, 2H), 2.65 (t, J=7.6 Hz, 2H), 1.85-1.74 (m, 1H), 1.68-1.57 (m, 2H), 1.39 (brs, 9H), 1.36-1.19 (m, 14H), 1.00-0.94 (m, 6H), 0.87 (t, J=6.6 Hz, 3H). ¹³C NMR (100 MHz, CDCl₃) δ 177.3, 168.2, 155.4, 146.5, 128.9, 127.4, 124.1, 79.4, 53.9, 35.9, 31.9, 31.5, 31.2, 29.9, 29.6, 29.6, 29.5, 29.3, 29.2, 28.3, 22.7, 19.4, 18.4, 14.1. HRMS: (ESI) [M+H]+ calc. for C₂₈H₄₆N₃O₃, 472.3534, observed, 472.3537.

### tert-butyl (2-(3-(4-decylphenyl)-1,2,4-oxadiazol-5-yl)ethyl)carbamate (4c)

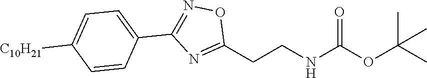

Synthesized according to General Procedure 4. Purified by silica chromatography (20% EtOAc in hexanes). Light yellow solid, 155 mg (50%) yield. ¹H NMR (400 MHz, CDCl₃) δ 7.97 (d, J=8.1 Hz, 2H), 7.27 (d, J=8.1 Hz, 2H), 5.27 (brs, 1H), 3.69-3.62 (m, 2H) 3.12 (t, J=6.0 Hz, 2H), 2.65 (t, J=7.7 hz, 2H), 1.68-1.58 (m, 2H), 1.43 (brs, 9H), 1.37-1.21 (m, 14H), 0.89 (t, J=6.8 Hz, 3H). ¹³C NMR (100 MHz, CDCl₃) δ 177.8, 168.3, 155.8, 146.6, 129.0, 127.4, 124.1, 79.7, 37.3, 36.0, 32.0, 31.3, 29.7, 29.6, 29.5, 29.4, 29.3, 28.4, 27.6, 22.8, 14.2. HRMS: (ESI) [M+H]+ calc. for C₂₅H₄₀N₃O₃, 430.3064, observed, 430.3076.

### tert-butyl (S)-(1-(3-(4-decylphenyl)-1,2,4-oxadiazol-5-yl)propan-2-yl)carbamate (4d)

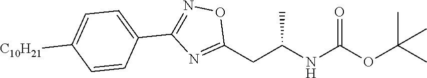

Synthesized by General Procedure 4. Purified by silica chromatography (15% EtOAc in hexanes). Yellow solid, 110 mg (46%) yield. ¹H NMR (400 MHz, CDCl₃) δ 7.98 (d, J=8.2 Hz, 2H), 7.28 (d, J=8.2 Hz, 2H), 5.02-4.90 (m, 1H), 4.29-4.17 (m, 1H), 3.15 (d, J=5.8 Hz, 2H), 2.66 (t, J=7.7 Hz, 2H), 1.68-1.57 (m, 2H), 1.43 (brs, 9H), 1.36-1.20 (m, 17H), 0.88 (t, J=6.8 Hz, 3H). ¹³C NMR (100 MHz, CDCl₃) δ 177.0, 168.3, 155.0, 146.6, 129.0, 127.5, 124.2, 79.7, 44.5, 36.0, 33.5, 32.0, 31.3, 29.7, 29.7, 29.6, 29.4, 20.3, 28.4, 22.8, 20.30, 14.2. HRMS: (ESI) [M+Na]+ calc. for C₂₆H₄₂N₃O₃, 444.3221, observed, 3244.

### (t)-tert-butyl 3-((3-(4-decylphenyl)-1,2,4-oxadiazol-5-yl)methyl)morpholine-4-carboxylate (4e)

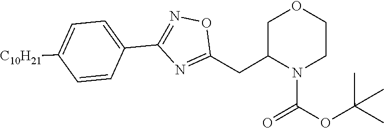

Synthesized according to General Procedure 4. Purified by silica chromatography (25% ethyl acetate in hexanes). Yellow solid (57%, 151 mg). ¹H NMR (400 MHz, CDCl₃) δ 7.94 (d, J=8.0 Hz, 2H), 7.24 (d, J=7.9 Hz, 2H), 4.59-4.33 (m, 1H), 4.02-3.70 (m, 3H), 3.63 (dd, J=11.8, 3.2 Hz, 1H), 3.54-3.18 (m, 4H), 2.62 (t, J=7.7 Hz, 2H), 1.61 (p, J=7.2 Hz, 2H), 1.41-1.15 (m, 23H), 0.84 (t, J=6.8 Hz, 3H). ¹³C NMR (101 MHz, CDCl₃) δ 176.9, 168.4, 154.1, 146.4, 128.9, 127.4, 124.2, 80.5, 68.9, 66.9, 50.4, 48.8, 40.0, 38.5, 36.0, 31.9, 31.3, 29.6, 29.6, 29.5, 29.4, 29.3, 28.2, 26.4, 22.7, 14.2. HRMS: (ESI) [M]+ calc. for C₂₈H₄₄N₃O₄, 486.3326, observed, 486.3342.

### tert-butyl (R)-2-(3-(4-decylphenyl)-1,2,4-oxadiazol-5-yl)morpholine-4-carboxylate (4f)

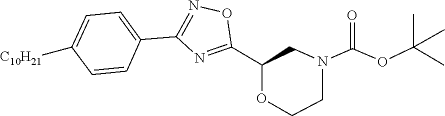

Synthesized according to General Procedure 4. Purified by silica chromatography (20 EtOAc in hexanes). Yellow oil, 246 mg (64%) yield. ¹H NMR (400 MHz, CDCl₃) δ 7.97 (d, J=8.2 Hz, 2H), 7.25 (d, J=8.2 Hz, 2H), 4.82-4.77 (m, 1H), 4.44-4.11 (m, 1H), 4.05 (d, J=11.1 Hz, 1H), 3.84 (d, J=13.3 Hz, 1H), 3.70 (t, J=10 Hz, 1H), 3.38 (brs, 1H), 3.23-3.13 (m, 1H), 2.61 (t, J=7.7 Hz, 2H), 1.65-1.54 (m, 2H), 1.45 (brs, 9H), 1.34-1.15 (m, 14H), 0.84 (t, J=6.8 Hz, 3H). ¹³C NMR (100 MHz, CDCl₃) δ 175.3, 168.4, 154.3, 146.7, 128.9, 127.5, 123.6, 80.7, 70.0, 66.5, 46.3, 43.1, 36.0, 31.9, 31.2, 29.6, 29.6, 29.5, 29.3, 29.3, 28.3, 22.7, 14.1. HRMS: (ESI) [M+H]+ calc. for C₂₇H₄₂N₃O₄, 472.3175, observed, 472.3204.

### tert-butyl ((1S,3R)-3-(3-(4-decylphenyl)-1,2,4-oxadiazol-5-yl)cyclopentyl)carbamate (4g)

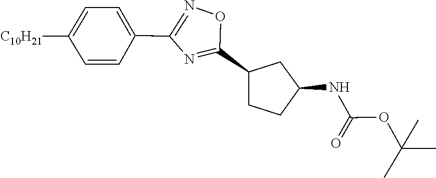

Synthesized according to General Procedure 4. Purified by silica chromatography (20% EtOAc in hexanes). Yellow solid, 213 mg (63%) yield. ¹H NMR (400 MHz, CDCl₃) δ 7.98 (d, J=8.2 Hz, 2H), 7.27 (d, J=8.2 Hz, 2H), 5.67 (brs, 1H), 4.23 (brs, 1H), 3.59-3.48 (m, 1H), 2.66 (t, J=7.7 Hz, 2H), 2.53-2.43 (m, 1H), 2.29-1.91 (m, 4H), 1.85-1.74 (m, 1H), 1.68-1.58 (m, 2H), 1.47 (brs, 9H), 1.38-1.19 (m, 14H), 0.88 (t, J=6.8 Hz, 3H). ¹³C NMR (100 MHz, CDCl₃) δ 183.6, 168.2, 155.5, 146.7, 129.0, 127.5, 124.1, 79.2, 52.0, 38.1, 36.1, 35.2, 33.4, 32.0, 31.4, 30.1, 29.7, 29.7, 29.6, 29.4, 29.4, 28.6, 28.6, 22.8, 14.3. HRMS: (ESI) [M+H]+ calc. for C₂₈H₄₄N₃O₃, 470.3377, observed, 470.3372.

### tert-butyl (3-(3-(4-decylphenyl)-1,2,4-oxadiazol-5-yl)propyl)carbamate (4h)

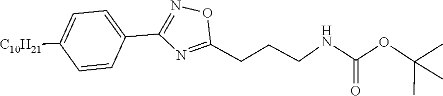

Synthesized according to General Procedure 4. Purified by silica chromatography (20% EtOAc in hexanes). Yellow oil, 260 mg (65%) yield. ¹H NMR (400 MHz, CDCl₃) δ 7.97 (d, J=8.2 Hz, 2H), 7.26 (d, J=8.2 Hz, 2H), 5.07 (brs, 1H), 3.32-3.22 (m, 2H), 2.97 (t, J=7.5 Hz, 2H), 2.64 (t, J=7.7 Hz, 2H), 2.11-2.02 (m, 2H), 1.67-1.57 (m, 2H), 1.43 (brs, 9H), 1.36-1.20 (m, 14H), 0.88 (t, J=6.8 Hz, 3H). ¹³C NMR (100 MHz, CDCl₃) δ 179.2, 168.2, 156.0, 146.4, 128.9, 127.3, 124.2, 79.2, 39.8, 35.9, 31.9, 31.2, 29.6, 29.6, 29.5, 29.3, 29.3, 28.4, 26.9, 24.1, 22.7, 14.1. HRMS: (ESI) [M+Na]+ calc. for C₂₆H₄₁N₃NaO₃, 466.3040, observed, 466.3034.

### tert-butyl ((3-(4-decylphenyl)-1,2,4-oxadiazol-5-yl)methyl)carbamate

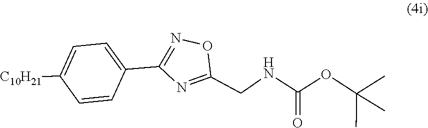

Synthesized according to General Procedure 4. Purified by silica chromatography (15% EtOAc in hexanes). White solid, 281 mg (75%) yield. ¹H NMR (400 MHz, CDCl₃) δ 7.95 (d, J=8.0 Hz, 2H), 7.26 (d, J=8.0 Hz, 2H), 5.59 (brs, 1H), 4.66-4.55 (m, 2H), 2.64 (t, J=7.7 Hz, 2H), 1.67-1.57 (m, 2H), 1.46 (brs, 9H), 1.36-1.20 (m, 14H), 0.88 (t, J=6.7 Hz, 3H). ¹³C NMR (100 MHz, CDCl₃) δ 176.5, 168.4, 155.6, 146.7, 128.9, 127.4, 123.9, 80.6, 37.2, 36.0, 32.0, 31.3, 29.7, 29.6, 29.5, 29.4, 29.4, 29.3, 28.3, 22.7, 14.2. HRMS: (ESI) [M+H]+ calc. for C₂₄H₃₈N₃O₃, 416.2908, observed, 416.2925.

### tert-butyl (3-(3-(4-decylphenyl)-1,2,4-oxadiazol-5-yl)-2-hydroxypropyl)carbamate

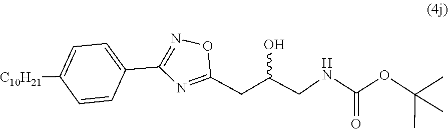

Synthesized according to General Procedure 4. Purified by silica chromatography (40% EtOAc in hexanes). Yellow solid, 452 mg (60%) yield. ¹H NMR (400 MHz, CDCl₃) δ 7.94 (d, J=8.0 Hz, 2H), 7.26 (d, J=8.0 Hz, 2H), 5.30 (brs, 1H), 4.30 (brs, 2H), 3.49-3.41 (m, 1H), 3.32-3.22 (m, 1H), 3.14-3.05 (m, 2H), 2.64 (t, J=7.7 Hz, 2H), 1.66-1.58 (m, 2H), 1.44 (brs, 9H), 1.36-1.20 (m, 14H), 0.88 (t, J=6.8 Hz, 3H). ¹³C NMR (100 MHz, CDCl₃) δ 177.3, 168.1, 156.9, 146.7, 129.0, 127.4, 123.8, 80.0, 68.5, 45.7, 36.0, 32.0, 31.7, 31.3, 29.7, 29.7, 29.6, 29.4, 29.3, 28.4, 22.8, 14.2. HRMS: (ESI) [M+H]+ calc. for C₂₆H₄₂N₃O₄, 460.3170, observed, 460.3169.

### tert-butyl 4-((3-(4-decylphenyl)-1,2,4-oxadiazol-5-yl)methyl)piperazine-1-carboxylate (4k)

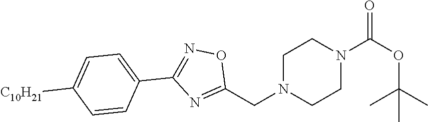

Synthesized according to General Procedure 4. Purified by silica chromatography (35% ethyl acetate in hexanes). White solid (63%, 248 mg). ¹H NMR (400 MHz, CDCl₃) δ 7.99 (d, J=8.2 Hz, 2H), 7.29 (d, J=8.2 Hz, 2H), 3.93 (s, 2H), 3.5 (t, J=5.0 Hz, 4H), 2.66 (t J=7.7 Hz, 2H), 2.60 (t J=5.0 Hz, 4H), 1.64 (p, J=7.3 Hz, 2H), 1.45 (s, 9H), 1.38-1.19 (m, 14H), 0.87 (t, J=6.7 Hz, 3H). ¹³C NMR (101 MHz, CDCl₃) δ 175.8, 168.5, 154.7, 146.8, 129.1, 127.6, 124.0, 80.0, 53.2, 52.7, 43.6, 36.1, 32.0, 31.4, 29.7, 29.7, 29.6, 29.5, 29.4, 28.5, 22.8, 14.3. HRMS: (ESI) [M+H]+ calc. for C₂₈H₄₅N₄O₃, 485.3486, observed, 485.3479.

### tert-butyl (3-(3-(4-(benzyloxy)phenyl)-1,2,4-oxadiazol-5-yl)propyl)carbamate (41)

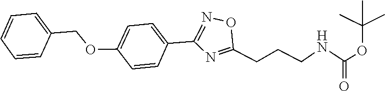 [SMILES: CC(C)(C)OC(=O)NCCCc1nc(-c2ccc(OCc3ccccc3)cc2)no1]

Synthesized according to General Procedure 4. Purified by silica chromatography (35% ethyl acetate in hexanes). White solid (31%, 585 mg). ¹H NMR (400 MHz, CDCl₃) δ 8.00 (d, J=8.9 Hz, 2H), 7.49-7.31 (m, 5H), 7.05 (d, J=8.9 Hz, 2H), 5.12 (s, 2H), 4.83 (brs, 1H), 3.35-3.22 (m, 2H), 2.98 (t, J=7.5 Hz, 2H), 2.06 (p, J=7.1 Hz, 2H), 1.44 (s, 9H). ¹³C NMR (101 MHz, CDCl₃) δ 179.2, 168.1, 161.2, 156.1, 136.5, 129.1, 128.8, 128.3, 127.6, 119.6, 115.2, 79.5, 70.2, 39.9, 28.5, 27.0, 24.2. HRMS: (ESI) [M+H]+ calc. for C₂₃H₂₈N₃O₄, 410.2074, observed, 410.2055.

### tert-butyl (3-(3-(3-decylphenyl)-1,2,4-oxadiazol-5-yl)propyl)carbamate (4m)

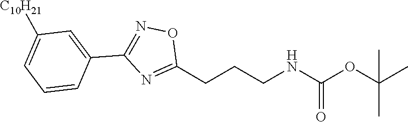

Synthesized according to General Procedure 4. Passed through a silica column silica (ethyl acetate in hexanes) to afford crude mixture which was carried forward without further purification.

### tert-butyl (E)-(3-(3-(4-decylphenyl)-1,2,4-oxadiazol-5-yl)allyl)carbamate (4n)

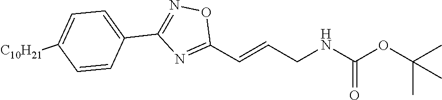

Synthesized according to General Procedure 4. Purified by silica chromatography (15% ethyl acetate in hexanes). White solid (74%, 143 mg). ¹H NMR (400 MHz, CDCl₃) δ 7.98 (d, J=8.2 Hz, 2H), 7.28 (d, J=8.1 Hz, 2H), 7.09 (dt, J=16.1, 4.9 Hz, 1H), 6.58 (d, J=16.1 Hz, 1H), 5.00 (brs, 1H), 4.03 (brs, 2H), 2.65 (t, J=7.7 Hz, 2H), 1.63 (p, J=7.1 Hz, 2H), 1.47 (s, 9H), 1.37-1.19 (m, 14H), 0.88 (t, J=6.7 Hz, 3H). ¹³C NMR (101 MHz, CDCl₃) δ 174.2, 168.7, 155.7, 146.6, 143.1, 129.0, 127.4, 124.2, 113.5, 80.0, 41.9, 36.0, 32.0, 31.3, 29.7, 29.7, 29.6, 29.4, 29.3, 28.4, 22.8, 14.2. HRMS: (ESI) [M+H]+ calc. for C₂₆H₄₀N₃O₃, 442.3070, observed, 442.3063.

### tert-butyl (3-(3-(4-decylphenyl)-1,2,4-oxadiazol-5-yl)propyl)(methyl)carbamate (4o)

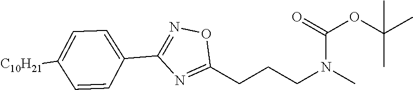

Synthesized according to General Procedure 4. Purified by silica chromatography (15% ethyl acetate in hexanes). Yellow oil (54%, 319 mg). ¹H NMR (400 MHz, CDCl₃) δ 7.96 (d, J=8.2 Hz, 2H), 7.28 (d, J=8.2 Hz, 2H), 3.38 (t, J=6.9 Hz, 2H), 2.94 (t, J=7.6 Hz, 2H), 2.88 (s, 3H), 2.65 (t, J=7.7 Hz, 2H), 2.10 (p, J=7.3 Hz, 2H), 1.63 (p, J=7.5 Hz, 2H), 1.44 (s, 9H), 1.38-1.19 (m, 14H), 0.87 (t, J=6.8 Hz, 3H). ¹³C NMR (101 MHz, CDCl₃) δ 179.0, 168.3, 146.4, 128.9, 127.3, 124.2, 79.3, 47.7, 35.9, 34.2, 31.9, 31.2, 29.6, 29.6, 29.5, 29.3, 29.2, 28.4, 24.7, 23.9, 22.7, 14.1. HRMS: (ESI) [M+H]+ calc. for C₂₇H₄₄N₃O₃, 458.3377, observed, 458.3372.

### 1-((tert-butoxycarbonyl)amino)-3-(3-(4-decylphenyl)-1,2,4-oxadiazol-5-yl)propan-2-yl acetate (4p)

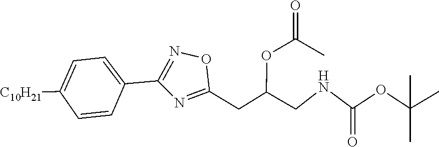

To a 6-dram vial containing 4j was added acetic anhydride (51 equiv) and TEA (9 equiv) and the mixture was allowed to stir at rt for 30 minutes. The mixture was then diluted with ethyl acetate and washed with a saturated sodium carbonate solution followed by a brine solution. The organic layer was then concentrated in vacuo and subjected to flash chromatography (25% ethyl acetate in hexanes) which afforded a crude mixture which was carried forward without any further purification.

### tert-butyl (3-(3-(4-decylphenyl)-1,2,4-oxadiazol-5-yl)benzyl)carbamate (4q)

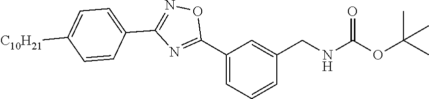

Synthesized according to General Procedure 4. Purified by silica chromatography (17% ethyl acetate in hexanes). Yellow solid (59%, 232 mg). ¹H NMR (400 MHz, CDCl₃) δ 8.14-8.09 (m, 2H), 8.07 (d, J=8.2 Hz, 2H), 7.56-7.49 (m, 2H), 7.32 (d, J=8.3 Hz, 2H), 4.99 (brs, 1H), 4.43 (d, J=6.2 Hz, 2H), 2.68 (t, J=7.5 Hz, 2H), 1.65 (p, J=7.4 Hz, 2H), 1.49 (s, 9H), 1.39-1.20 (m, 14H), 0.87 (t, J=6.8 Hz, 3H). ¹³C NMR (101 MHz, CDCl₃) δ 175.5, 169.1, 156.0, 146.7, 140.5, 131.8, 129.6, 129.1, 127.6, 127.2, 127.0, 124.8, 124.4, 80.0, 44.4, 36.1, 32.1, 31.4, 29.8, 29.7, 29.6, 29.5, 29.4, 28.5, 22.8, 14.3. HRMS: (ESI) [M+H]+ calc. for C₃₀H₄₂N₃O₃, 492.3221, observed, 492.3209.

### tert-butyl ((±)-cis-3-(3-(4-decylphenyl)-1,2,4-oxadiazol-5-yl)cyclohexyl)carbamate (4r)

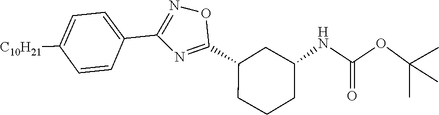

Synthesized according to General Procedure 4. Purified by silica chromatography (12% ethyl acetate in hexanes). Yellow solid (72%, 314 mg). ¹H NMR (400 MHz, CDCl₃) δ 7.96 (d, J=8.2 Hz, 2H), 7.27 (d, J=8.2 Hz, 2H), 4.58 (s, 1H), 3.61 (s, 1H), 3.10 (dt, J=11.6, 5.9 Hz, 1H), 2.71-2.58 (m, 2H), 2.48 (d, J=12.4 Hz, 1H), 2.15 (d, J=9.4 Hz, 1H), 2.05 (d, J=12.7 Hz, 1H), 1.99-1.90 (m, 1H), 1.70-1.49 (m, 6H), 1.45 (s, 9H), 1.38-1.13 (m, 14H), 0.87 (t, J=6.8 Hz, 3H). ¹³C NMR (101 MHz, CDCl₃) δ 181.5, 168.2, 155.0, 146.4, 128.9, 127.3, 124.2, 79.3, 48.8, 36.6, 35.9, 35.5, 32.6, 31.9, 31.2, 29.6, 29.6, 29.5, 29.3, 29.2, 28.4, 24.1, 22.7, 14.1. HRMS: (ESI) [M+Na]+ calc. for C₂₉H₄₅N₃NaO₃, 506.3353, observed, 506.3337.

### tert-butyl ((±)-trans-3-(3-(4-decylphenyl)-1,2,4-oxadiazol-5-yl)cyclohexyl)carbamate (4s)

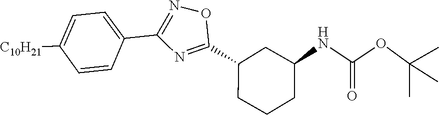

Synthesized according to General Procedure 4. Purified by silica chromatography (12% ethyl acetate in hexanes). Yellow solid (73%, 318 mg). ¹H NMR (400 MHz, CDCl₃) δ 7.97 (d, J=8.2 Hz, 2H), 7.27 (d, J=8.3 Hz, 2H), 4.63 (brs, 1H), 3.95 (brs 1H), 3.29 (brs, 1H), 2.65 (d, J=7.7 Hz, 2H), 2.22-1.54 (m, 10H), 1.45 (s, 9H), 1.28 (d, J=19.6 Hz, 14H), 0.87 (t, J=6.8 Hz, 3H). ¹³C NMR (101 MHz, CDCl₃) δ 181.9, 168.4, 155.2, 146.5, 129.0, 127.5, 124.4, 79.5, 45.5, 36.1, 34.5, 32.2, 32.0, 31.4, 30.9, 29.7, 29.7, 29.6, 29.5, 29.4, 29.2, 28.6, 22.8, 20.6, 14.2. HRMS: (ESI) [M+Na]+ calc. for C₂₉H₄₅N₃NaO₃, 506.3353, observed, 506.3336.

### tert-butyl (3-(3-(4-bromobenzyl)-1,2,4-oxadiazol-5-yl)propyl)carbamate (4t)

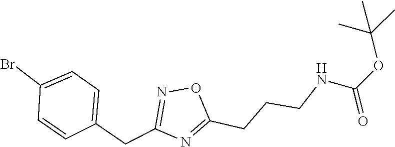 [SMILES: CC(C)(C)OC(=O)NCCCc1nc(Cc2ccc(Br)cc2)no1]

Synthesized according to General Procedure 4. Purified by silica chromatography (35% ethyl acetate in hexanes). Yellow oil (68%, 292 mg). ¹H NMR (400 MHz, CDCl₃) δ 7.44 (d, J=8.4 Hz, 2H), 7.19 (d, J=8.5 Hz, 2H), 4.65 (s, 1H), 3.99 (s, 2H), 3.21 (q, J=6.3 Hz, 2H), 2.89 (t, J=7.6 Hz, 2H), 1.97 (p, J=7.0 Hz, 2H), 1.43 (s, 9H). ¹³C NMR (101 MHz, CDCl₃) δ 179.7, 169.1, 156.0, 134.5, 132.0, 130.8, 121.3, 79.6, 39.8, 31.9, 28.5, 27.0, 24.1. HRMS: (ESI) [M+H]+ calc. for C₁₇H₂₃BrN₃O₃, 418.0737, observed, 418.0746.

### tert-butyl (3-(3-(4-nonylbenzyl)-1,2,4-oxadiazol-5-yl)propyl)carbamate (4u)

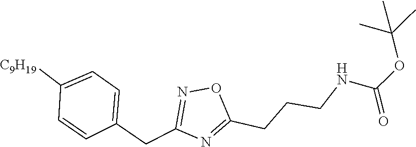

Synthesized according to General Procedure 1 from 4ag. Purified by silica chromatography (25% ethyl acetate in hexanes). Colorless oil (48%, 149 mg). ¹H NMR (400 MHz, CDCl₃) δ 7.21 (d, J=8.0 Hz, 2H), 7.12 (d, J=8.1 Hz, 2H), 4.69 (s, 1H), 4.00 (s, 2H), 3.20 (q, J=6.2 Hz, 2H), 2.88 (t, J=7.6 Hz, 2H), 2.56 (d, J=7.7 Hz, 2H), 1.97 (p, J=7.0 Hz, 2H), 1.58 (p, J=7.4 Hz, 2H), 1.43 (s, 9H), 1.27 (d, J=14.6 Hz, 12H), 0.86 (t, 3H). ¹³C NMR (101 MHz, CDCl₃) δ 179.4, 169.7, 156.0, 141.9, 132.7, 128.9, 128.9, 79.5, 39.8, 35.7, 32.0, 32.0, 31.6, 29.7, 29.6, 29.5, 29.4, 28.5, 27.0, 24.1, 22.8, 14.2. HRMS: (ESI) [M+Na]+ calc. for C₂₆H₄₁N₃O₃Na, 466.3040, observed, 466.3049.

### tert-butyl (3-(3-(4-bromophenethyl)-1,2,4-oxadiazol-5-yl)propyl)carbamate (4v)

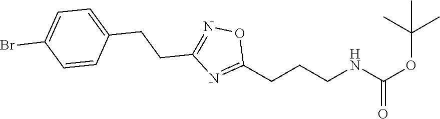 [SMILES: CC(C)(C)OC(=O)NCCCc1nc(CCc2ccc(Br)cc2)no1]

Synthesized according to General Procedure 4. Purified by silica chromatography (35% ethyl acetate in hexanes). Yellow oil (76%, 322 mg). ¹H NMR (400 MHz, CDCl₃) δ 7.40 (d, J=8.3 Hz, 2H), 7.08 (d, J=8.3 Hz, 2H), 4.71 (s, 1H), 3.22 (q, J=6.2 Hz, 2H), 3.00 (q, J=3.1, 2.2 Hz, 4H), 2.90 (t, J=7.5 Hz, 2H), 1.99 (p, J=7.1 Hz, 2H), 1.43 (s, 9H). ¹³C NMR (101 MHz, CDCl₃) δ 179.3, 169.6, 156.1, 139.3, 131.7, 130.2, 120.3, 79.6, 39.8, 32.5, 28.5, 27.7, 27.0, 24.0. HRMS: (ESI) [M+Na]+ calc. for C₁₈H₂₄BrN₃O₃Na, 432.0893, observed, 432.0894.

### tert-butyl (3-(3-(4-octylphenethyl)-1,2,4-oxadiazol-5-yl)propyl)carbamate (4w)

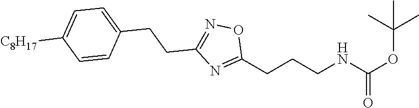

Synthesized according to General Procedure 1 from 4ai. Purified by silica chromatography (20% ethyl acetate in hexanes). Colorless oil (50%, 166 mg). ¹H NMR (400 MHz, CDCl₃) δ 7.16-7.06 (m, 4H), 4.72 (s, 1H), 3.23 (q, J=6.3 Hz, 2H), 3.05-2.98 (m, 4H), 2.91 (t, J=7.5 Hz, 2H), 2.56 (t, 2H), 2.01 (p, J=6.9 Hz, 2H), 1.58 (p, J=7.5 Hz, 2H), 1.44 (s, 9H), 1.38-1.20 (m, 10H), 0.87 (d, J=6.8 Hz, 3H). ¹³C NMR (101 MHz, CDCl₃) δ 179.1, 170.1, 156.0, 141.1, 137.6, 128.7, 128.3, 79.5, 39.8, 35.7, 32.8, 32.0, 31.7, 29.6, 29.5, 29.4, 28.5, 28.1, 27.0, 24.0, 22.8, 14.2. HRMS: (ESI) [M+Na]+ calc. for C₂₆H₄₁N₃O₃Na, 466.3040, observed, 466.3044.

### tert-butyl (3-(3-(4-(nonyloxy)phenyl)-1,2,4-oxadiazol-5-yl)propyl)carbamate (4x)

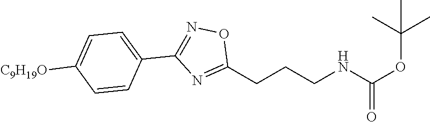

Synthesized according to General Procedure 4. Purified by silica chromatography (15% ethyl acetate in hexanes). White solid (79%, 316 mg). ¹H NMR (400 MHz, CDCl₃) δ 7.98 (d, J=9.0 Hz, 2H), 6.96 (d, J=9.0 Hz, 2H), 4.81 (s, 1H), 4.00 (t, J=6.6 Hz, 2H), 3.28 (q, J=6.1 Hz, 2H), 2.97 (t, J=7.4 Hz, 2H), 2.06 (p, J=7.0 Hz, 2H), 1.88-1.71 (m, 2H), 1.44 (s, 11H), 1.36-1.26 (m, 10H), 0.87 (t, 3H). ¹³C NMR (101 MHz, CDCl₃) δ 179.1, 168.2, 161.7, 156.1, 129.1, 119.1, 114.9, 68.3, 39.9, 32.0, 29.7, 29.5, 29.4, 29.3, 28.5, 27.1, 26.2, 24.2, 22.8, 14.2. HRMS: (ESI) [M+H]+ calc. for C₂₅H₄₀N₃O₄, 446.3013, observed, 446.3004.

### tert-butyl (3-(3-(4-(nonyloxy)-3-(trifluoromethyl)phenyl)-1,2,4-oxadiazol-5-yl)propyl)carbamate (4y)

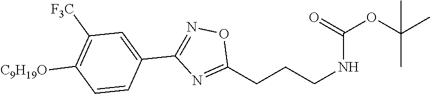

Synthesized according to General Procedure 4. Purified by silica chromatography (20% ethyl acetate in hexanes). Yellow solid (57%, 260 mg). ¹H NMR (400 MHz, CDCl₃) δ 1H NMR (400 MHz, Chloroform-d) δ 8.3 (d, J=1.9 Hz, 1H), 8.2 (dd, J=8.7, 2.0 Hz, 1H), 7.1 (d, J=8.7 Hz, 1H), 4.7 (s, 1H), 4.1 (t, J=6.4 Hz, 2H), 3.3 (q, J=6.1 Hz, 2H), 3.0 (t, J=7.5 Hz, 2H), 2.1 (p, J=7.0 Hz, 2H), 1.9-1.8 (m, 2H), 1.4 (s, 11H), 1.4-1.2 (m, 10H), 0.9 (t, J=6.8 Hz, 3H). ¹³C NMR (101 MHz, CDCl₃) δ 179.6, 167.4, 159.3, 132.5, 126.8 (q, J=5.3 Hz), 123.4 (q, J=272.6 Hz), 119.6 (q, J=31.4 Hz), 118.7, 113.0, 79.6, 69.2, 39.9, 32.0, 29.6, 29.3, 29.3, 29.0, 28.5, 27.1, 25.9, 24.1, 22.8, 14.2. HRMS: (ESI) [M+H]+ calc. for C₂₆H₃₉F₃N₃O₄, 514.2887, observed, 514.2881.

### tert-butyl (3-(3-(3-fluoro-4-(nonyloxy)phenyl)-1,2,4-oxadiazol-5-yl)propyl)carbamate (4z)

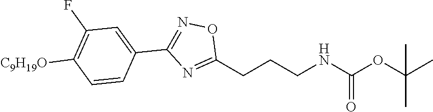

Synthesized according to General Procedure 4. Purified by silica chromatography (20% ethyl acetate in hexanes). Yellow solid (87%, 360 mg). ¹H NMR (400 MHz, CD₃OD) δ 7.8-7.8 (m, 2H), 7.0 (t, J=8.6 Hz, 1H), 4.8 (s, 1H), 4.1 (t, J=6.6 Hz, 2H), 3.3 (q, J=6.1 Hz, 2H), 3.0 (t, J=7.4 Hz, 2H), 2.1 (p, J=7.0 Hz, 2H), 1.9-1.8 (m, 2H), 1.4 (s, 11H), 1.4-1.2 (m, 10H), 0.9 (t, 3H). ¹³C NMR (101 MHz, CD₃OD) δ HRMS: (ESI) [M+H]+ calc. for C₂₅H₃₉FN₃O₄, 464.2919, observed, 464.2910.

### 3-tert-butyl (3-(3-(6-(heptyloxy)naphthalen-2-yl)-1,2,4-oxadiazol-5-yl)propyl)carbamate (4aa)

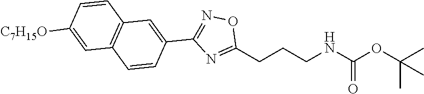

Synthesized according to General Procedure 4. Purified by silica chromatography (20% ethyl acetate in hexanes). White solid (68%, 265 mg). ¹H NMR (400 MHz, CDCl₃) δ 8.49 (s, 1H), 8.06 (dd, J=8.6, 1.7 Hz, 1H), 7.82 (d, J=9.0 Hz, 1H), 7.78 (d, J=8.6 Hz, 1H), 7.18 (dd, J=8.9, 2.5 Hz, 1H), 7.14 (d, J=2.4 Hz, 1H), 4.82 (s, 1H), 4.08 (t, J=6.6 Hz, 2H), 3.35-3.23 (m, 2H), 3.01 (t, J=7.5 Hz, 2H), 2.10 (p, J=7.1 Hz, 2H), 1.85 (p, J=6.7 Hz, 2H), 1.55-1.25 (m, 17H), 0.90 (t, J=6.8 Hz, 3H). ¹³C NMR (101 MHz, CDCl₃) δ 179.3, 168.6, 158.6, 156.1, 136.3, 130.4, 128.4, 127.8, 127.5, 124.4, 121.8, 120.0, 106.7, 79.5, 68.2, 39.9, 29.3, 29.2, 28.5, 27.1, 26.2, 24.2, 22.8, 14.2. HRMS: (ESI) [M+H]+ calc. for C₂₇H₃₈N₃O₄, 468.2857, observed, 468.2857.

### tert-butyl (R)-3-(3-(6-(heptyloxy)naphthalen-2-yl)-1,2,4-oxadiazol-5-yl)pyrrolidine-1-carboxylate (4ab)

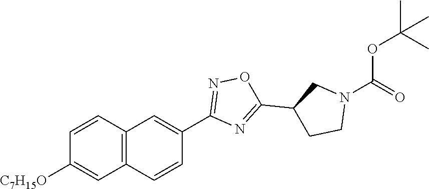

Synthesized according to General Procedure 4. Purified by silica chromatography (20% ethyl acetate in hexanes). Yellow solid (76%, 305 mg). ¹H NMR (400 MHz, CDCl₃) δ 8.51 (brs, 1H), 8.07 (dd, J=8.6, 1.7 Hz, 1H), 7.83 (d, J=8.9 Hz, 1H), 7.79 (d, J=8.6 Hz, 1H), 7.20 (dd, J=8.9, 2.5 Hz, 1H), 7.15 (d, J=2.4 Hz, 1H), 4.09 (t, J=6.6 Hz, 2H), 3.97-3.44 (m, 5H), 2.48-2.32 (m, 2H), 1.89-1.81 (m, 2H), 1.54-1.27 (m, 17H), 0.89 (t, J=6.8 Hz, 3H). ¹³C NMR (101 MHz, CDCl₃) δ 168.7, 158.7, 136.3, 130.4, 128.5, 127.9, 127.6, 124.4, 121.7, 120.1, 106.7, 79.9, 68.3, 49.3, 45.3, 36.8, 36.0, 31.9, 30.6, 29.8, 29.3, 29.2, 28.6, 26.2, 22.8, 14.2. HRMS: (ESI) [M+H]+ calc. for C₂₈H₃₈N₃O₄, 480.2857, observed, 480.2854.

### 3-(4-decylphenyl)-5-(piperidin-4-yl)-1,2,4-oxadiazole 2,2,2-trifluoroacetate (5a)

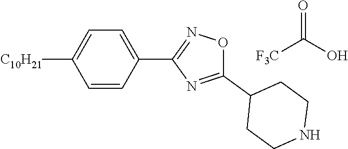

Synthesized according to General Procedure 5. Purified by silica chromatography (7% MeOH in DCM). White solid, 330 mg (89%) yield. ¹H NMR (400 MHz, CD₃OD) δ 7.96 (d, J=8.3 Hz, 2H), 7.33 (d, J=8.3 Hz, 2H), 3.56-3.43 (m, 3H), 3.27-3.16 (m, 2H), 2.68 (t, J=7.7 Hz, 2H), 2.48-2.35 (m, 2H), 2.21-2.06 (m, 2H), 1.73-1.57 (m, 2H), 1.42-1.16 (m, 14H), 0.89 (t, J=6.9 Hz, 3H). ¹³C NMR (100 MHz, CD₃OD) δ 181.5, 169.5, 148.1, 130.1, 128.3, 125.3, 71.4, 44.0, 36.9, 33.1, 32.9, 32.5, 30.7, 30.6, 30.5, 30.3, 27.2, 23.7, 14.5. HRMS: (ESI) [M+H]+ calc. for C₂₃H₃₆N₃O, 370.2853, observed, 370.2826.

### 2-(3-(4-decylphenyl)-1,2,4-oxadiazol-5-yl)ethan-1-amine 2,2,2-trifluoroacetate (5b)

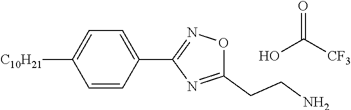

Synthesized according to General Procedure 5. White solid, 110 mg (76%) yield. ¹H NMR (400 MHz, CD₃OD) δ 7.99 (d, J=8.2 Hz, 2H), 7.34 (d, J=8.2 Hz, 2H), 3.52 (t, J=6.8 Hz, 2H), 3.38 (t, J=6.7 Hz, 2H), 2.68 (t, J=7.7 Hz, 2H), 1.70-1.60 (m, 2H), 1.40-1.22 (m, 14H), 0.89 (t, J=6.8 Hz, 3H). ¹³C NMR (100 MHz, CD₃OD) δ 177.6, 169.6, 148.2, 130.1, 28.4, 125.2, 37.3, 36.9, 33.1, 32.5, 30.7, 30.6, 30.5, 30.3, 25.4, 23.7, 14.5. HRMS: (ESI) [M+H]+ calc. for C₂₀H₃₂N₃O, 330.254, observed, 330.2529.

### (S)-1-(3-(4-decylphenyl)-1,2,4-oxadiazol-5-yl)propan-2-amine 2,2,2-trifluoroacetate (5c)

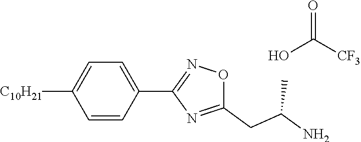

Synthesized according to General Procedure 5. White solid, 76 mg (67%) yield. ¹H NMR (400 MHz, CD₃OD) δ 7.98 (d, J=8.1 Hz, 2H), 7.31 (d, J=8.1 Hz, 2H), 4.03-3.87 (m, 1H), 3.36 (d, J=6.3 Hz, 2H), 2.66 (t, J=7.7 Hz, 2H), 1.71-1.56 (m, 2H), 1.47 (d, J=6.7 Hz, 3H), 1.39-1.20 (m, 14H), 0.88 (t, J=6.7 Hz, 3H). ¹³C NMR (100 MHz, CD₃OD) δ 177.0, 169.6, 163.1 (q, J=34.7 Hz), 148.1, 130.1, 128.4, 125.1, 46.6, 36.9, 33.1, 32.4, 32.0, 30.7, 30.6, 30.5, 30.3, 23.7, 18.7, 14.5. HRMS: (ESI) [M+H]+ calc. for C₂₁H₃₄N₃O, 344.2696, observed, 344.2705.

### (1S,3R)-3-(3-(4-decylphenyl)-1,2,4-oxadiazol-5-yl)cyclopentan-1-amine hydrochloride (5d)

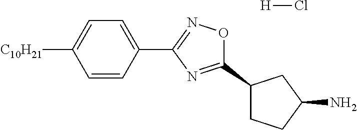

Synthesized according to General Procedure 7. White solid, 122 mg (64%) yield. ¹H NMR (400 MHz, CD₃OD) δ 7.95 (d, J=8.3 Hz, 2H), 7.32 (d, J=8.3 Hz, 2H), 3.82 (p, J=7.5 Hz, 1H), 3.66 (p, J=7.5 Hz, 1H), 2.78-2.64 (m, 3H), 2.38-2.07 (m, 4H), 1.98-1.88 (m, 1H), 1.70-1.59 (m, 2H), 1.38-1.21 (m, 14H), 0.89 (t, J=6.8 Hz, 3H). ¹³C NMR (100 MHz, CD₃OD) δ 183.1, 169.4, 148.0, 130.1, 128.3, 125.4, 52.6, 36.9, 36.8, 36.8, 36.8, 33.1, 32.5, 31.3, 30.7, 30.6, 30.5, 30.3, 30.3, 23.7, 14.5. HRMS: (ESI) [M+H]+ calc. for C₂₃H₃₆N₃O, 370.2853, observed, 370.2859.

### 3-(3-(4-decylphenyl)-1,2,4-oxadiazol-5-yl)propan-1-amine 2,2,2-trifluoroacetate (5e)

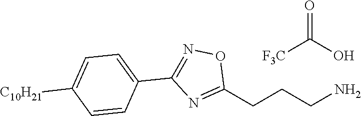

Synthesized according to General Procedure 5. Purified by silica chromatography (10% MeOH in DCM). White solid, 160 mg (63%) yield. ¹H NMR (400 MHz, CD₃OD) δ 7.93 (d, J=8.1 Hz, 2H), 7.29 (d, J=8.1 Hz, 2), 3.19-3.07 (m, 4H), 2.64 (t, J=7.6 Hz, 2H), 2.25 (p, J=7.5 Hz, 2H), 1.69-1.58 (m, 2H), 1.37-1.20 (m, 14H), 0.87 (t, J=6.6 Hz, 3H). ¹³C NMR (100 MHz, CD₃OD) δ 180.1, 169.4, 147.9, 130.0, 128.3, 125.3, 39.8, 36.8, 33.0, 32.4, 30.7, 30.6, 30.4, 30.3, 25.1, 24.3, 23.7, 14.5. HRMS: (ESI) [M+H]+ calc. for C₂₁H₃₄N₃O, 344.2696, observed, 344.2701.

### (3-(4-decylphenyl)-1,2,4-oxadiazol-5-yl)methanamine 2,2,2-trifluoroacetate (5f)

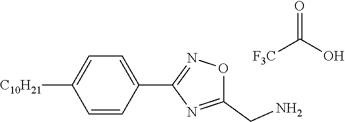

Synthesized according to General Procedure 5. Purified by silica chromatography (10% MeOH in DCM). White solid, 250 mg (86%) yield. ¹H NMR (400 MHz, CD₃OD) δ 7.99 (d, J=7.8 Hz, 2H), 7.31 (d, J=7.8 Hz, 2H), 4.60 (brs, 2H), 2.66 (t, J=7.7 Hz, 2H), 1.70-1.57 (m, 2H), 1.39-1.20 (m, 14H), 0.88 (t, J=6.8 Hz, 3H). ¹³C NMR (100 MHz, CD₃OD) δ 174.4, 169.7, 148.4, 130.1, 128.5, 124.7, 36.9, 36.1, 33.0, 32.4, 30.7, 30.6, 30.4, 30.3, 23.7, 14.5. HRMS: (ESI) [M+H]+ calc. for C₁₉H₃₀N₃O, 316.2383, observed, 316.2393.

### 1-amino-3-(3-(4-decylphenyl)-1,2,4-oxadiazol-5-yl)propan-2-ol hydrochloride (5g)

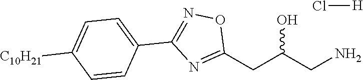

Synthesized according to General Procedure 6. White solid, 298 mg (87%) yield. ¹H NMR (400 MHz, CD₃OD) δ 7.95 (d, J=8.3 Hz, 2H), 7.33 (d, J=8.3 Hz, 2H), 4.43-4.34 (m, 1H), 3.30=3.15 (m, 3H), 3.09-3.01 (m, 1H), 2.67 (t, J=7.7 Hz, 2H), 1.70-1.60 (m, 2H), 1.38-1.21 (m, 14H), 0.88 (t, J=6.9 Hz, 3H). ¹³C NMR (100 MHz, CD₃OD) δ 178.2, 169.6, 148.1, 130.1, 128.3, 125.4, 66.5, 45.2, 36.9, 33.3, 33.1, 32.5, 30.7, 30.6, 30.5, 30.3, 23.7, 14.5.

### 3-(4-decylphenyl)-5-(piperazin-1-ylmethyl)-1,2,4-oxadiazole 2,2,2-trifluoroacetate (5h)

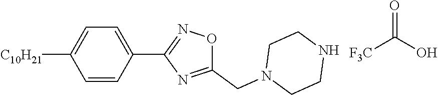

Synthesized according to General Procedure 6. White solid (49%, 100 mg). ¹H NMR (400 MHz, CD₃OD) δ 7.91 (d, J=8.0 Hz, 2H), 7.27 (d, J=8.0 Hz, 2H), 4.03 (s, 2H), 4.03-3.16 (m, 4H), 2.96-2.82 (m, 4H), 2.61 (t J=7.7 Hz, 2H), 1.63-1.53 (m, 2H), 1.42-1.45 (m, 14H), 0.84 (t, J=6.7 Hz, 3H). ¹³C NMR (101 MHz, CD₃OD) δ 177.2 169.5, 148.1, 130.1, 128.4, 125.2, 122.9, 53.0, 50.2, 44.8, 36.8, 33.1, 32.4, 30.7, 30.6, 30.5, 30.3, 23.7, 14.5. HRMS: (ESI) [M+H]+ calc. for C₂₃H₃₇N₄O, 385.2962, observed, 385.2980.

### 3-(3-(4-(benzyloxy)phenyl)-1,2,4-oxadiazol-5-yl)propan-1-amine hydrochloride (5i)

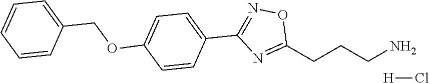

Synthesized according to General Procedure 6. White solid (89%, 120 mg). ¹H NMR (400 MHz, CDCl₃) δ 7.98 (d, J=9.0 Hz, 2H), 7.55-7.27 (m, 5H), 7.13 (d, J=9.1 Hz, 2H), 5.16 (s, 2H), 3.19-3.06 (m, 4H), 2.23 (p, J=7.7 Hz, 2H). ¹³C NMR (101 MHz, CDCl₃) δ 180.0, 169.2, 162.8, 138.2, 129.9, 129.6, 129.1, 128.6, 120.4, 116.4, 71.1, 39.9, 25.2, 24.3. HRMS: (ESI) [M+H]+ calc. for C₁₈H₂₀N₃O₂, 310.1550, observed, 310.1530.

### 3-(3-(3-decylphenyl)-1,2,4-oxadiazol-5-yl)propan-1-amine hydrochloride (5j)

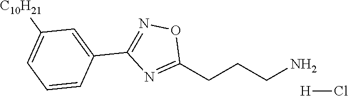

Synthesized according to General Procedure 6. White solid (53%, 90 mg). ¹H NMR (400 MHz, CD₃OD) δ 7.87-7.81 (m, 2H), 7.38 (t, J=7.6 Hz, 1H), 7.32 (d, J=7.6 Hz, 1H), 3.21-3.09 (m, 4H), 2.64 (t, J=7.7 Hz, 2H), 2.27 (p, J=7.5 Hz, 2H), 1.62 (p, J=7.2 Hz, 2H), 1.39-1.17 (m, 14H), 0.87 (t, J=6.7 Hz, 3H). ¹³C NMR (101 MHz, CD₃OD) δ 180.2, 169.5, 144.9, 132.5, 129.9, 128.1, 127.8, 125.7, 39.8, 36.7, 33.0, 32.6, 30.7, 30.6, 30.4, 30.3, 28.8, 25.1, 24.3, 23.7, 14.5. HRMS: (ESI) [M+H]+ calc. for C₂₁H₃₄N₃O, 344.2696, observed, 344.2685.

### (±)-3-chloro-3-(3-(4-decylphenyl)-1,2,4-oxadiazol-5-yl)propan-1-amine hydrochloride (5k)

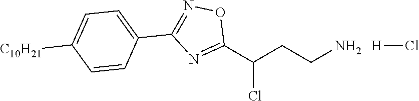

Synthesized according to General Procedure 6. White solid (58%, 54 mg). ¹H NMR (400 MHz, CDCl₃) δ 7.96 (d, J=8.2 Hz, 2H), 7.34 (d, J=8.2 Hz, 2H), 4.84-4.76 (m, 1H), 3.72-3.55 (m, 3H), 3.44 (dd, J=13.7, 9.9 Hz, 1H), 2.69 (t, J=7.7 Hz, 2H), 1.66 (p, J=7.7 Hz, 2H), 1.43-1.18 (m, 14H), 0.89 (t, J=6.5 Hz, 3H). ¹³C NMR (101 MHz, CDCl₃) δ 176.9, 169.7, 148.2, 130.1, 128.3, 125.2, 55.6, 46.0, 36.8, 33.9, 33.1, 32.4, 30.7, 30.5, 30.4, 30.3, 23.7, 14.4. HRMS: (ESI) [M+H]+ calc. for C₂₁H₃₃ClN₃O, 378.2312, observed, 378.2312.

### 3-(3-(4-decylphenyl)-1,2,4-oxadiazol-5-yl)-N,N-dimethylpropan-1-amine hydrochloride (51)

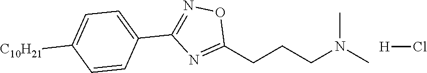

Amine free base of the title compound was prepared according to General Procedure 4. The title compound was prepared by dissolving the amine free base in methanolic HCl followed by concentration in vacuo. White solid (33%, 80 mg). ¹H NMR (400 MHz, CDCl₃) δ 7.96 (d, J=8.2 Hz, 2H), 7.34 (d, J=7.34 (d, J=8.2 Hz, 2H), 3.40-3.30 (m, 2H), 3.14 (t, J=7.3 Hz, 2H), 2.97 (s, 6H), 2.69 (t, J=7.7 Hz 2H), 2.40-2.29 (m, 2H), 1.66 (p, J=7.2 Hz, 2H), 1.42-1.22 (m, 14H), 0.90 (t, J=6.8 Hz, 3H). ¹³C NMR (101 MHz, CDCl₃) δ 179.9, 169.5, 148.0, 130.1, 128.3, 125.3, 57.8, 43.6, 36.8, 33.0, 32.4, 30.7, 30.5, 30.4, 30.3, 24.2, 23.7, 22.4, 14.4. HRMS: (ESI) [M+H]+ calc. for C₂₃H₃₈N₃O, 372.3009, observed, 372.3005.

### 3-(3-(4-decylphenyl)-1,2,4-oxadiazol-5-yl)-N-methylpropan-1-amine hydrochloride (5m)

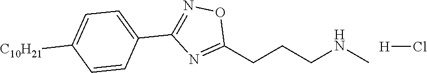

Synthesized according to General Procedure 6. White solid (79%, 211 mg). ¹H NMR (400 MHz, CDCl₃) δ 7.95 (d, J=7.8 Hz, 2H), 7.34 (d, J=7.7 Hz, 2H), 3.20 (t, J=7.6 Hz, 2H), 3.13 (t, J=7.1 hz, 2H), 2.75 (s, 3H), 2.68 (t, J=7.6 Hz, 2H), 2.35-2.20 (m, 2H), 1.71-1.59 (m, 2H), 1.42-1.20 (m, 14H), 0.89 (t, J=6.4 Hz, 3H). ¹³C NMR (101 MHz, CDCl₃) δ 179.9, 169.4, 147.9, 130.0, 128.3, 125.3, 49.7, 36.8, 34.4, 33.0, 32.4, 30.6, 30.5, 30.3, 30.2, 24.6, 24.0, 23.6, 14.4. HRMS: (ESI) [M+H]+ calc. for C₂₂H₃₆N₃O, 358.2853, observed, 358.2850.

### 1-amino-3-(3-(4-decylphenyl)-1,2,4-oxadiazol-5-yl)propan-2-yl acetate hydrochloride (5n)

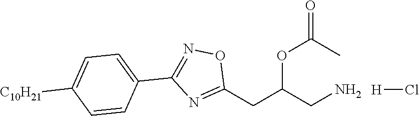

Synthesized according to General Procedure 6. White solid (71%, 93 mg). ¹H NMR (400 MHz, CDCl₃) δ 7.96 (d, J=7.8 Hz, 2H), 7.35 (d, J=7.9 Hz, 2H), 5.57 (q, J=7.5, 7.1 Hz, 1H), 3.59-3.35 (m, 4H), 2.69 (t, J=7.6 Hz, 2H), 2.12 (s, 3H), 1.66 (q, J=7.5 Hz, 2H), 1.32 (d, J=22.8 Hz, 14H), 0.90 (t, J=6.6 Hz, 3H). ¹³C NMR (101 MHz, CDCl₃) δ 176.9, 171.9, 169.6, 148.2, 130.1, 128.3, 125.2, 68.8, 43.1, 36.8, 33.1, 32.4, 30.7, 30.5, 30.4, 30.3, 30.0, 23.7, 20.8, 14.4 HRMS: (ESI) [M+H]+ calc. for C₂₃H₃₆N₃O₃, 402.2751, observed, 402.2747.

### (3-(3-(4-decylphenyl)-1,2,4-oxadiazol-5-yl)phenyl)methanamine hydrochloride (5o)

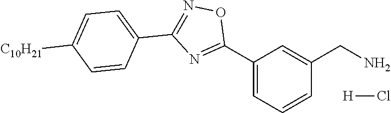

Synthesized according to General Procedure 6. White solid (91%, 174 mg). ¹H NMR (400 MHz, CD₃OD) δ 8.37 (s, 1H), 8.29 (dt, J=7.7, 1.5 Hz, 1H), 8.05 (d, J=8.2 Hz, 2H), 7.79 (dt, J=7.8, 1.6 Hz, 1H), 7.73 (t, J=7.7 Hz, 1H), 7.38 (d, J=8.3 Hz, 2H), 4.28 (s, 2H), 2.71 (t, J=7.5 Hz, 2H), 1.66 (p, J=7.2 Hz, 2H), 1.44-1.21 (m, 14H), 0.88 (t, J=6.8 Hz, 3H). ¹³C NMR (101 MHz, CD₃OD) δ 174.6, 167.0, 148.9, 136.4, 134.6, 131.4, 130.2, 129.7, 129.6, 128.4, 126.3, 125.4, 43.9, 36.9, 34.2, 32.4, 30.7, 30.6, 30.4, 30.3, 23.1, 14.0. HRMS: (ESI) [M+H]+ calc. for C₂₅H₃₄N₃O, 392.2696, observed, 392.2698.

### (f)-cis-3-(3-(4-decylphenyl)-1,2,4-oxadiazol-5-yl)cyclohexan-1-amine hydrochloride (5p)

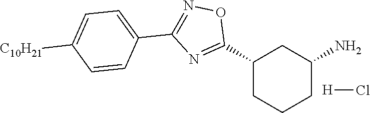

Synthesized according to General Procedure 6. White solid (87%, 226 mg). ¹H NMR (400 MHz, CD₃OD) δ 7.95 (d, J=8.2 Hz, 2H), 7.32 (d, J=8.2 Hz, 2H), 3.71-3.52 (m, J=20.7, 10.2, 5.1 Hz, 2H), 2.67 (t, J=7.7 Hz, 2H), 2.61-2.51 (m, 1H), 2.26-2.16 (m, 1H), 2.10-1.77 (m, 4H), 1.71-1.51 (m, 4H), 1.40-1.20 (m, 14H), 0.88 (t, J=6.8 Hz, 3H). ¹³C NMR (101 MHz, CD₃OD) δ 182.4, 169.5, 148.0, 130.1, 128.3, 125.4, 48.0, 36.8, 33.3, 33.1, 32.9, 32.4, 30.8, 30.7, 30.5, 30.4, 30.3, 28.5, 23.7, 21.3, 14.4. HRMS: (ESI) [M+H]+ calc. for C₂₄H₃₈N₃O, 384.3009, observed, 384.2967.

### (±)-trans-3-(3-(4-decylphenyl)-1,2,4-oxadiazol-5-yl)cyclohexan-1-amine hydrochloride (5q)

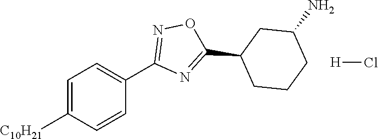

Synthesized according to General Procedure 6. White solid (74%, 192 mg). ¹H NMR (400 MHz, CD₃OD) δ 7.93 (d, J=8.3 Hz, 2H), 7.31 (d, J=8.3 Hz, 2H), 3.39-3.32 (m, 1H), 3.29-3.19 (m, 1H), 2.66 (d, J=7.8 Hz, 2H), 2.57-2.50 (m, 1H), 2.28-2.19 (m, 1H), 2.17-2.09 (m, 1H), 2.08-2.01 (m, 1H), 1.77 (q, J=12.3 Hz, 1H), 1.70-1.41 (m, 5H), 1.39-1.21 (m, 14H), 0.88 (d, J=6.8 Hz, 3H). ¹³C NMR (101 MHz, CD₃OD) δ 182.6, 169.5, 148.0, 130.1, 128.3, 125.4, 50.5, 36.8, 35.9, 34.8, 33.0, 32.4, 31.0, 30.7, 30.5, 30.4, 30.3, 30.1, 24.4, 23.7, 14.4. HRMS: (ESI) [M+H]+ calc. for C₂₄H₃₈N₃O, 384.3009, observed, 384.2970.

### 3-(3-(4-nonylbenzyl)-1,2,4-oxadiazol-5-yl)propan-1-amine hydrochloride (5r)

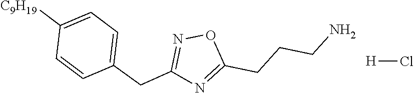

Synthesized according to General Procedure 6. White solid (45%, 50 mg). ¹H NMR (400 MHz, CD₃OD) δ 7.19 (d, J=8.0 Hz, 2H), 7.12 (d, J=8.0 Hz, 2H), 4.01 (s, 2H), 3.13-2.97 (m, 4H), 2.57 (d, J=7.8 Hz, 2H), 2.13 (p, J=7.5 Hz, 2H), 1.58 (d, J=7.5 Hz, 2H), 1.40-1.22 (m, 12H), 0.89 (t, J=6.8 Hz, 3H). ¹³C NMR (101 MHz, CD₃OD) δ 178.8, 169.5, 141.5, 132.7, 128.4, 128.3, 38.4, 35.1, 31.6, 31.3, 31.0, 29.3, 29.2, 29.0, 28.9, 23.6, 22.8, 22.3, 13.0. HRMS: (ESI) [M+H]+ calc. for C₂₁H₃₄N₃O, 344.2696, observed, 344.2703.

### 3-(3-(4-octylphenethyl)-1,2,4-oxadiazol-5-yl)propan-1-amine hydrochloride (5s)

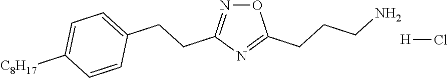

Synthesized according to General Procedure 6. White solid (46%, 49 mg). ¹H NMR (400 MHz, CD₃OD) δ 7.13-7.06 (m, 4H), 3.07 (dt, J=14.5, 7.5 Hz, 4H), 3.00 (s, 4H), 2.55 (d, J=7.7 Hz, 2H), 2.17 (p, J=7.5 Hz, 2H), 1.59 (p, J=7.5, 6.6 Hz, 2H), 1.37-1.21 (m, 10H), 0.89 (t, J=6.8 Hz, 3H). ¹³C NMR (101 MHz, CD₃OD) δ 179.9, 171.1, 142.1, 138.8, 129.6, 129.2, 39.8, 36.5, 33.5, 33.0, 32.8, 30.6, 30.4, 30.3, 28.7, 25.1, 24.2, 23.7, 14.4. HRMS: (ESI) [M+H]+ calc. for C₂₁H₃₄N₃O, 344.2696, observed, 344.2700.

### 3-(3-(4-(nonyloxy)phenyl)-1,2,4-oxadiazol-5-yl)propan-1-amine hydrochloride (5t)

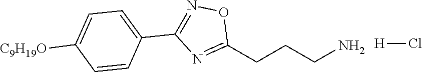

Synthesized according to General Procedure 6. White solid (88%, 226 mg). ¹H NMR (400 MHz, CD₃OD) δ 7.96 (d, J=9.0 Hz, 2H), 7.03 (d, J=9.0 Hz, 2H), 4.04 (t, J=6.4 Hz, 2H), 3.17-3.08 (m, 4H), 2.29-2.17 (m, 2H), 1.85-1.74 (m, 2H), 1.55-1.44 (m, 2H), 1.44-1.23 (m, 10H), 0.93-0.86 (m, 3H). ¹³C NMR (101 MHz, CD₃OD) δ 180.0, 169.3, 163.2, 129.9, 120.0, 115.9, 69.3, 39.9, 33.0, 30.7, 30.5, 30.4, 30.3, 27.1, 25.2, 24.3, 23.7, 14.4. HRMS: (ESI) [M+H]+ calc. for C₂₀H₃₂N₃O₂, 346.2489, observed, 346.2480.

### 3-(3-(4-(nonyloxy)-3-(trifluoromethyl)phenyl)-1,2,4-oxadiazol-5-yl)propan-1-amine hydrochloride (5u)

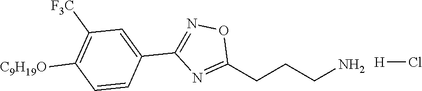

Synthesized according to General Procedure 6. White solid (72%, 155 mg). ¹H NMR (400 MHz, CD₃OD) δ 8.25-8.20 (m, 2H), 7.30 (d, J=9.2 Hz, 1H), 4.17 (t, J=6.2 Hz, 2H), 3.22-3.11 (m, 4H), 2.26 (p, J=7.5 Hz, 2H), 1.87-1.75 (m, 2H), 1.52 (p, J=7.0 Hz, 2H), 1.41-1.26 (m, 10H), 0.89 (d, J=6.8 Hz, 3H). ¹³C NMR (101 MHz, CD₃OD) δ 180.5, 168.4, 160.7 (q, J=1.6 Hz), 133.8, 127.0 (q, J=5.5 Hz), 124.8 (q, J=271.8 Hz), 120.2 (q, J=31.3 Hz), 119.8, 114.8, 70.3, 39.8, 33.0, 30.6, 30.3, 30.3, 30.0, 26.9, 25.1, 24.3, 23.7, 14.4. HRMS: (ESI) [M+H]+ calc. for C₂₁H₃₁F₃N₃O₂, 414.2363, observed, 414.2355.

### 3-(3-(3-fluoro-4-(nonyloxy)phenyl)-1,2,4-oxadiazol-5-yl)propan-1-amine hydrochloride (5v)

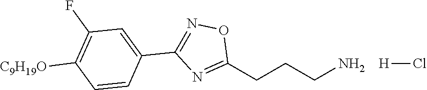

Synthesized according to General Procedure 6. White solid (79%, 236 mg). ¹H NMR (400 MHz, CD₃OD) δ 7.8 (dt, J=8.6, 1.5 Hz, 1H), 7.7 (dd, J=11.9, 2.0 Hz, 1H), 7.2 (t, J=8.5 Hz, 1H), 4.1 (t, J=6.4 Hz, 2H), 3.1 (q, J=7.4 Hz, 2H), 2.2 (p, J=7.4 Hz, 2H), 1.9-1.7 (m, 2H), 1.6-1.4 (m, 2H), 1.4-1.2 (m, 10H), 0.9 (d, J=6.8 Hz, 3H). ¹³C NMR (101 MHz, CD₃OD) δ 180.3, 168.6 (d, J=2.6 Hz), 153.7 (d, J=245.8 Hz), 151.2 (d, J=10.7 Hz), 125.1 (d, J=3.7 Hz), 120.5 (d, J=7.4 Hz), 115.8, 115.7 (d, J=23.9 Hz), 70.4, 39.9, 33.0, 30.6, 30.4, 30.4, 30.2, 27.0, 25.2, 24.3, 23.7, 14.4. HRMS: (ESI) [M+H]+ calc. for C₂₀H₃₁FN₃O₂, 364.2395, observed, 364.2387.

### 3-(3-(6-(heptyloxy)naphthalen-2-yl)-1,2,4-oxadiazol-5-yl)propan-1-amine hydrochloride (5w)

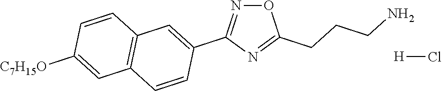

Synthesized according to General Procedure 6. White solid (85%, 166 mg). ¹H NMR (400 MHz, CD₃OD) δ 8.50 (d, J=1.6 Hz, 1H), 8.03 (dd, J=8.6 Hz, 1H), 7.88 (d, J=3.5 Hz, 1H), 7.86 (d, J=3.8 Hz, 1H), 7.29 (d, J=2.4 Hz, 1H), 7.21 (dd, J=9.0 Hz, 2.5 Hz, 1H), 4.13 (t, J=6.4 Hz, 2H), 3.21-3.11 (m, 4H), 2.27 (p, J=7.4 Hz, 2H), 1.91-1.81 (m, 2H), 1.58-1.49 (m, 2H), 1.47-1.31 (m, 6H), 0.93 (t, J=6.8 Hz, 3H). HRMS: (ESI) [M+H]+ calc. for C₂₁H₃₁F₃N₃O₂, 414.2363, observed, 414.2355.

### (R)-3-(6-(heptyloxy)naphthalen-2-yl)-5-(pyrrolidin-3-yl)-1,2,4-oxadiazole hydrochloride (5x)

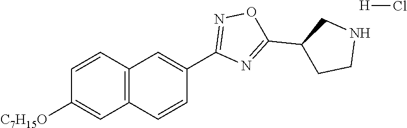

Synthesized according to General Procedure 6. White solid (75%, 175 mg). ¹H NMR (400 MHz, CDCl₃) δ 10.3 (brs, 1H), 8.52 (d, J=1.7 Hz, 1H), 8.04 (dd, J=8.6, 1.7 Hz, 1H), 7.83 (d, J=9.0 Hz, 1H), 7.77 (d, J=8.6 Hz, 1H), 7.18 (dd, J=8.9, 2.5 Hz, 1H), 7.12 (d, J=2.5 Hz, 1H), 4.07 (t, J=6.6 Hz, 2H), 4.01-3.91 (m, 2H), 3.88-3.78 (m, 2H), 3.69-3.58 (m, 2H), 2.71-2.59 (m, 1H), 2.55-2.43 (m, 1H), 1.90-1.79 (m, 2H), 1.55-1.44 (m, 2H), 1.43-1.25 (m, 6H), 0.90 (t, J=6.8 Hz, 3H). ¹³C NMR (101 MHz, CDCl₃) δ 180.0, 169.8, 160.1, 137.9, 131.2, 129.7, 128.7, 125.0, 122.6, 121.1, 111.4, 107.7, 69.2, 49.5, 46.6, 36.7, 33.0, 30.4, 30.3, 30.2, 27.2, 23.7, 14.4. HRMS: (ESI) [M+H]+ calc. for C₂₃H₃₀N₃O₂, 380.2333, observed, 380.2331.

### (R)-2-amino-4-(3-(4-decylphenyl)-1,2,4-oxadiazol-5-yl)butanoic acid hydrochloride (5y)

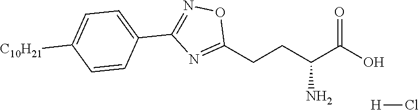

Synthesized according to general procedure 4 with the following adaptation. The Boc-protected product was then subjected to General Procedure 6. White solid (69%, 145 mg). ¹H NMR (400 MHz, CD₃OD) δ 7.90 (d, J=8.2 Hz, 2H), 7.27 (d, J=8.1 Hz, 2H), 4.19 (t, J=6.6 Hz, 1H), 3.24-3.11 (m, 2H), 2.61 (t J=7.7 Hz, 2H), 2.55-2.35 (m, 2H), 1.59 (p, J=7.2 Hz, 2H), 1.35-1.15 (m, 14H), 0.83 (t, J=6.7 Hz, 3H). ¹³C NMR (101 MHz, CD₃OD) δ 179.7, 171.1, 169.5, 148.0, 130.1, 128.3, 125.3, 53.0, 36.8, 33.1, 32.4, 30.7, 30.6, 30.4, 30.3, 28.1, 28.0, 23.7, 23.5, 14.5. HRMS: (ESI) [M+H]+ calc. for C₂₂H₃₄N₃O₃, 388.2595, observed, 388.2595.

### 1-(3-(4-decylphenyl)-1,2,4-oxadiazol-5-yl)-3-(piperidin-1-yl)propan-2-ol hydrochloride (6a)

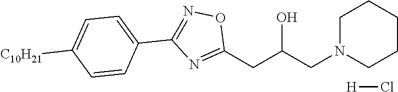

Synthesized according to General Procedure 7 from 5g. Purified by silica chromatography (10% methanol in dichloromethane). White solid (19%, 10 mg). ¹H NMR (400 MHz, CD₃OD) δ 7.96 (d, J=8.3 Hz, 2H), 7.33 (d, J=8.2 Hz, 2H), 4.68-4.59 (m, 1H), 3.64 (d, J=11.9 Hz, 1H), 3.56 (d, J=12.5 Hz, 1H), 3.39 (dd, J=13.3, 3.0 Hz, 1H), 3.27-2.95 (m, 5H), 2.68 (t, J=7.7 Hz, 2H), 2.02-1.47 (m, 8H), 1.40-1.19 (m, 14H), 0.88 (t, J=6.7 Hz, 3H). ¹³C NMR (101 MHz, CD₃OD) δ 176.5, 168.2, 146.7, 128.7, 126.9, 123.9, 62.5, 60.4, 54.8, 51.7, 35.4, 32.1, 31.6, 31.0, 29.3, 29.1, 29.0, 28.9, 22.4, 22.4, 22.3, 21.2, 13.0. HRMS: (ESI) [M+H]+ calc. for C₂₆H₄₂N₃O₂, 428.3272, observed, 428.3264.

### 1-(3-(4-decylphenyl)-1,2,4-oxadiazol-5-yl)-3-(pyrrolidin-1-yl)propan-2-ol hydrochloride (6b)

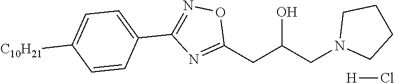

Synthesized according to General Procedure 7 from 5g. Purified by silica chromatography (5-15% methanol in dichloromethane). White solid (40%, 30 mg). ¹H NMR (400 MHz, CD₃OD) δ 7.96 (d, J=8.2 Hz, 2H), 7.34 (d, J=8.2 Hz, 2H), 4.55-4.47 (m, 1H), 3.39-3.32 (m, 6H), 3.28-3.14 (m, 2H), 2.68 (t, J=7.7 Hz, 2H), 2.11-2.03 (m, 4H), 1.65 (p, J=7.4 Hz, 2H), 1.40-1.22 (m, 14H), 0.89 (t, J=6.8 Hz, 3H). ¹³C NMR (101 MHz, CD₃OD) δ 178.2, 169.6, 148.1, 130.1, 128.3, 125.4, 65.8, 60.7, 55.5, 36.8, 33.6, 33.1, 32.5, 30.7, 30.6, 30.5, 30.3, 24.0, 23.7, 14.4. HRMS: (ESI) [M+H]+ calc. for C₂₅H₄₀N₃O₂, 414.3115, observed, 414.3133.

### 1-(3-(4-decylphenyl)-1,2,4-oxadiazol-5-yl)-3-morpholinopropan-2-ol hydrochloride (6c)

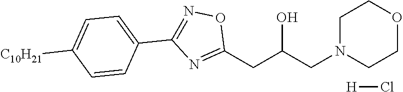

Synthesized according to General Procedure 7 from 5g. Purified by silica chromatography (5-10% methanol in dichloromethane). White solid (64%, 84 mg). ¹H NMR (400 MHz, CD₃OD) δ 7.96 (d, J=8.3 Hz, 2H), 7.33 (d, J=8.2 Hz, 2H), 4.73-4.65 (m, 1H), 4.11-3.98 (m, 2H), 3.93-3.76 (m, 2H), 3.65-3.52 (m, 2H), 3.51-3.63 (m, 2H), 3.34-3.17 (m, 4H), 2.68 (t, J=7.7 Hz, 2H), 1.64 (p, J=7.4 Hz, 2H), 1.39-1.21 (m, 14H), 0.89 (t, J=6.7 Hz, 3H). ¹³C NMR (101 MHz, CD₃OD) δ ¹³C NMR (101 MHz, CD₃OD) δ 177.9, 169.6, 148.06, 130.1, 128.3, 125.4, 64.7, 63.7, 62.2, 54.8, 52.3, 36.8, 33.5, 33.1, 32.5, 30.7, 30.6, 30.5, 30.3, 23.7, 14.5. HRMS: (ESI) [M+H]+ calc. for C₂₅H₄₀N₃O₃, 430.3064, observed, 430.3061.

### 3-(4-decylphenyl)-5-(3-(pyrrolidin-1-yl)propyl)-1,2,4-oxadiazole hydrochloride (6d)

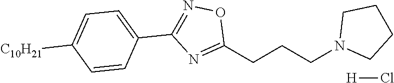

Synthesized according to General Procedure 7 from 5e. Purified by silica chromatography (5-10% methanol in dichloromethane). White solid (68%, 56 mg). ¹H NMR (400 MHz, CD₃OD) δ 7.95 (d, J=8.2 Hz, 2H), 7.33 (d, J=8.2 Hz, 2H), 3.40-3.29 (m, 6H), 3.13 (t, J=7.3 Hz, 2H), 2.68 (t, J=7.7 Hz, 2H), 2.37-2.27 (m, 2H), 2.14-2.04 (m, 4H), 1.65 (p, J=7.3 Hz, 2H), 1.40-1.21 (m, 14H), 0.89 (t, J=6.8 Hz, 3H). ¹³C NMR (101 MHz, CD₃OD) δ 180.1, 169.5, 148.1, 130.1, 128.3, 125.4, 55.3, 55.1, 36.8, 33.1, 32.5, 30.7, 30.6, 30.5, 30.3, 24.4, 24.0, 24.0, 23.7, 14.5. HRMS: (ESI) [M+H]+ calc. for C₂₅H₄₀N₃O, 398.3166, observed, 398.3155.

### 3-(4-decylphenyl)-5-(3-(piperidin-1-yl)propyl)-1,2,4-oxadiazole hydrochloride (6e)

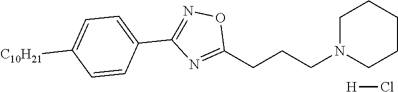

Synthesized according to General Procedure 7 from 5e. Purified by silica chromatography (5-10% methanol in dichloromethane). White solid (38%, 32 mg). ¹H NMR (400 MHz, CD₃OD) δ 7.94 (d, J=8.3 Hz, 2H), 7.31 (d, J=8.2 Hz, 2H), 3.05 (t, J=7.3 Hz, 2H), 2.94-2.75 (m, 6H), 2.66 (t, J=7.7 Hz, 2H), 2.20 (p, J=7.5 Hz, 2H), 1.72 (p, J=5.7 Hz, 2H), 1.69-1.51 (m, 4H), 1.39-1.18 (m, 14H), 0.89 (t, J=6.7 Hz, 3H). ¹³C NMR (101 MHz, CD₃OD) δ 180.7, 169.4, 147.9, 130.1, 128.3, 125.5, 58.2, 55.0, 36.9, 33.1, 32.5, 30.7, 30.6, 30.5, 30.3, 25.5, 24.9, 24.0, 23.8, 23.2, 14.5. HRMS: (ESI) [M+H]+ calc. for C₂₆H₄₂N₃O, 412.3322, observed, 412.3315.

### 4-(3-(3-(4-decylphenyl)-1,2,4-oxadiazol-5-yl)propyl)morpholine hydrochloride (6f)

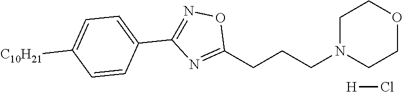

Synthesized according to General Procedure 7 from 5e. Purified by silica chromatography (5-10% methanol in dichloromethane). White solid (73%, 62 mg). ¹H NMR (400 MHz, CD₃OD) δ 7.95 (d, J=8.2 Hz, 2H), 7.33 (d, J=8.1 Hz, 2H), 4.13-3.76 (m, 4H), 3.63-3.48 (m, 2H), 3.41-3.33 (m, 2H), 3.27-3.17 (m, 2H), 3.14 (t, J=7.3 Hz, 2H), 2.68 (t, J=7.7 Hz, 2H), 2.43-2.32 (m, 2H), 1.65 (p, J=7.2 Hz, 2H), 1.39-1.21 (m, 14H), 0.89 (t, J=6.8 Hz, 3H). ¹³C NMR (101 MHz, CD₃OD) δ 179.9, 169.5, 148.1, 130.1, 128.3, 125.4, 65.1, 57.3, 63.3, 36.8, 33.1, 32.5, 30.7, 30.6, 30.5, 30.3, 24.3, 23.7, 21.6, 14.5. HRMS: (ESI) [M+H]+ calc. for C₂₅H₄₀N₃O₂, 414.3115, observed, 414.3113.

### 1-(3-(4-decylphenyl)-1,2,4-oxadiazol-5-yl)-3-(ethylamino)propan-2-ol hydrochloride (7a)

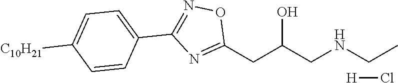

To a round bottom flask containing amine salt 5g was added methanol (0.1 M), glacial acetic acid (2 equiv), and sodium cyanoborohydride at rt. Acetaldehyde (1.2 equiv) was then added and the mixture stirred overnight (ca. 16 hours) at rt. The reaction mixture was diluted in dichloromethane and washed with 2M sodium bicarbonate solution and brine. The aqueous layer was washed with dichloromethane 3×. The combined organic layers were dried over sodium sulfate and concentrated. Purified by silica chromatography (10% methanol in dichloromethane). The product was then dissolved in methanolic HCl and concentrated to afford the title compound as an HCl salt. White solid (27%, 20 mg). ¹H NMR (400 MHz, CD₃OD) δ 7.96 (d, J=7.9 Hz, 2H), 7.34 (d, J=7.9 Hz, 2H), 4.54-4.41 (m, 1H), 3.39-3.06 (m, 5H), 2.68 (t, J=7.8 Hz, 2H), 1.65 (p, J=7.2 Hz, 2H), 1.41-1.20 (m, 17H), 0.89 (t, J=6.8 Hz, 3H). ¹³C NMR (101 MHz, CD₃OD) δ 178.1, 169.6, 148.1, 130.1, 128.3, 125.4, 65.7, 52.5, 44.1, 36.8, 33.4, 33.1, 32.5, 30.7, 30.6, 30.5, 30.3, 23.7, 14.5, 11.4. HRMS: (ESI) [M+H]+ calc. for C₂₃H₃₈N₃O₂, 388.2959, observed, 388.2977.

### 1-(3-(4-decylphenyl)-1,2,4-oxadiazol-5-yl)-3-(dimethylamino)propan-2-ol hydrochloride (7b)

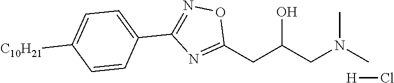

To a round bottom flask was added amine salt 5g (1 equiv) followed by paraformaldehyde (10 equiv) and methanol (0.2M). Sodium borohydride (6 equiv) was then added and the mixture was heated to reflux overnight (ca. 16 hours). The reaction mixture was diluted in dichloromethane and washed with 2M sodium bicarbonate solution and brine. The aqueous layer was washed with dichloromethane 3x. The combined organic layers were dried over sodium sulfate and concentrated. Purified by silica chromatography (0-15% methanol in dichloromethane). The product was then dissolved in methanolic HCl and concentrated to afford the title compound as an HCl salt. White solid (20%, 16 mg). ¹H NMR (400 MHz, CD₃OD) δ 7.96 (d, J=8.3 Hz, 2H), 7.34 (d, J=8.2 Hz, 2H), 4.53-4.44 (m, 1H), 3.22 (dd, J=15.4, 5.0 Hz, 1H), 3.14 (dd, J=15.4, 7.0 Hz, 1H), 3.09-3.04 (m, 2H), 2.73 (s, 6H), 2.68 (t, J=7.7 Hz, 2H), 1.65 (p, J=7.6 hz, 2H), 1.39-1.22 (m, 14H), 0.89 (t, J=6.8 Hz, 3H). ¹³C NMR (101 MHz, CD₃OD) δ 178.4, 169.6, 148.0, 130.1, 128.3, 125.4, 65.4 63.7, 44.6, 36.8, 33.6, 33.1, 32.5, 30.7, 30.6, 30.5, 30.3, 23.7, 14.4. HRMS: (ESI) [M+H]+ calc. for C₂₃H₃₈N₃O₂, 388.2959, observed, 388.2953.

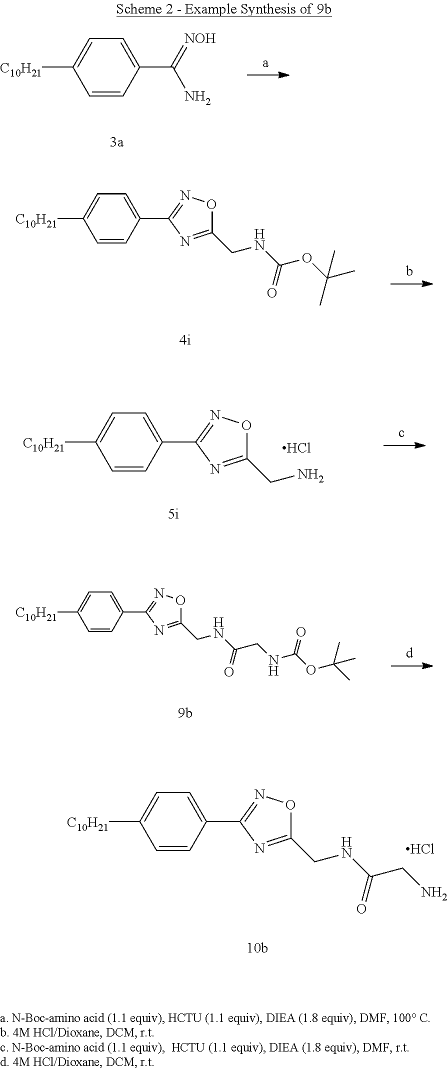

### tert-butyl (S)-(1-(((3-(4-decylphenyl)-1,2,4-oxadiazol-5-yl)methyl)amino)-1-oxopropan-2-yl)carbamate (8a)

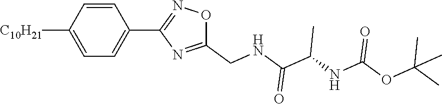

Synthesized according to General Procedure 8. Purified via column chromatography (35% ethyl acetate/hexanes). White solid (91%, 252 mg). ¹H NMR (400 MHz, CDCl₃) δ 7.91 (d, J=8.3 Hz, 2H), 7.55 (brs, 1H), 7.24 (d, J=8.2 Hz, 2H), 5.39 (d, J=7.7 Hz, 1H), 4.72 (d, J=5.7 Hz, 2H), 4.34 (brs, 1H), 2.62 (t, J=7.7 Hz, 2H), 1.61 (p, J=7.2 Hz, 2H), 1.44-1.37 (s, 12H), 1.33-1.22 (m, 14H), 0.86 (t, J=6.8 Hz, 3H). ¹³C NMR (101 MHz, CDCl₃) δ 175.81, 173.50, 168.46, 155.84, 146.75, 128.96, 127.47, 123.81, 80.39, 50.02, 36.04, 35.80, 31.98, 31.28, 29.69, 29.66, 29.56, 29.41, 29.36, 28.38, 22.76, 18.30, 14.20.

### tert-butyl (2-(((3-(4-decylphenyl)-1,2,4-oxadiazol-5-yl)methyl)amino)-2-oxoethyl)carbamate (8b)

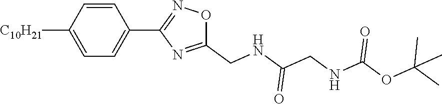

Synthesized according to General Procedure 8. Purified by silica chromatography (35% ethyl acetate in hexanes). Yellow oil (99%, 268 mg). ¹H NMR (400 MHz, Chloroform-d) δ 7.92 (d, J=8.3 Hz, 2H), 7.39 (s, 1H), 7.26 (d, J=10.4 Hz, 2H), 5.51 (s, 1H), 4.76 (s, 2H), 3.93 (s, 2H), 2.64 (t, J=7.7 Hz, 2H), 1.61 (p, J=7.1 Hz, 2H), 1.44 (s, 9H), 1.35-1.22 (m, 14H), 0.88 (t, J=6.7 Hz, 3H). ¹³C NMR (101 MHz, CDCl₃) δ 175.67, 170.31, 168.52, 156.40, 146.87, 129.01, 127.50, 123.73, 80.62, 44.38, 36.06, 35.76, 32.00, 31.30, 29.71, 29.69, 29.58, 29.43, 29.39, 28.39, 22.79, 14.22.

### tert-butyl 4-(2-(((3-(4-decylphenyl)-1,2,4-oxadiazol-5-yl)methyl)amino)-2-oxoethyl)piperazine-1-carboxylate (8c)

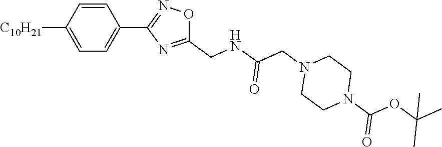

Synthesized according to General Procedure 8. Purified via column chromatography (80% ethyl acetate/hexanes). Clear oil (82%, 190 mg). ¹H NMR (400 MHz, CDCl₃) δ 7.93 (d, J=8.0 Hz, 2H), 7.87 (t, J=5.9 Hz, 1H), 7.29 (d, J=7.8 Hz, 2H), 4.80 (d, J=5.9 Hz, 2H), 3.51 (t, J=5.0 Hz, 4H), 3.14 (s, 2H), 2.66 (t, J=7.4 Hz, 2H), 2.58 (t, J=5.1 Hz, 4H), 1.63 (p, J=7.4 Hz, 2H), 1.47 (s, 9H), 1.38-1.20 (m, 14H), 0.88 (t, J=6.8 Hz, 3H). ¹³C NMR (101 MHz, CDCl₃) δ 175.94, 170.47, 168.50, 154.71, 146.93, 129.09, 127.43, 123.77, 80.07, 61.48, 53.38, 43.94, 36.07, 35.40, 32.00, 31.29, 29.71, 29.66, 29.57, 29.42, 29.37, 28.51, 22.78, 14.22.

### tert-butyl (4-(((3-(4-decylphenyl)-1,2,4-oxadiazol-5-yl)methyl)amino)-4-oxobutyl)carbamate (8d)

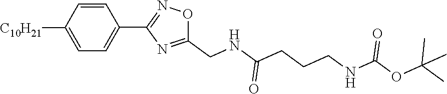

Synthesized according to General Procedure 8. Purified by silica chromatography (35-50% ethyl acetate in hexanes). Yellow oil (87%, 124 mg). ¹H NMR (400 MHz, Methanol-d₄) δ 7.92 (d, J=8.0 Hz, 2H), 7.29 (d, J=8.0 Hz, 2H), 4.66 (s, 2H), 3.10 (t, J=6.9 Hz, 2H), 2.65 (t, J=7.7 Hz, 2H), 2.32 (t, J=7.5 Hz, 2H), 1.80 (p, J=7.2 Hz, 2H), 1.62 (p, J=7.3 Hz, 2H), 1.41 (s, 9H), 1.34-1.23 (m, 14H), 0.87 (t, J=6.7 Hz, 3H). ¹³C NMR (101 MHz, Methanol-d₄) δ 178.31, 176.05, 169.51, 158.51, 147.98, 130.06, 128.34, 125.26, 79.92, 40.71, 36.85, 36.46, 33.95, 33.07, 32.43, 30.71, 30.70, 30.57, 30.46, 30.31, 28.78, 27.08, 23.74, 14.47.

### tert-butyl (3-(((3-(4-decylphenyl)-1,2,4-oxadiazol-5-yl)methyl)amino)-3-oxopropyl)carbamate (8e)

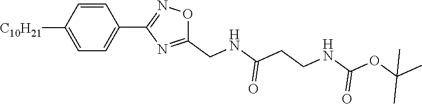

Synthesized according to General Procedure 8. Purified via column chromatography (35% ethyl acetate/hexanes). White solid (65%, 225 mg). ¹H NMR (400 MHz, CDCl₃) δ 7.94 (d, J=8.2 Hz, 2H), 7.27 (d, J=7.9 Hz, 2H), 6.95 (brs, 1H), 5.39 (brs, 1H), 4.73 (d, J=5.7 Hz, 2H), 3.46 (q, J=6.2 Hz, 2H), 2.65 (t, J=7.7 Hz, 2H), 2.53 (t, J=6.0 Hz, 2H), 1.63 (p, 2H), 1.41 (s, 9H), 1.37-1.20 (m, 14H), 0.88 (t, J=6.7 Hz, 3H). ¹³C NMR (101 MHz, CDCl₃) δ 175.96, 172.09, 168.47, 156.36, 146.87, 129.04, 127.51, 123.75, 79.61, 36.75, 36.43, 36.06, 35.88, 32.00, 31.30, 29.70, 29.68, 29.58, 29.42, 29.37, 28.46, 22.78, 14.21.

### tert-butyl (S)-(1-(((3-(4-decylphenyl)-1,2,4-oxadiazol-5-yl)methyl)amino)-3-methyl-1-oxobutan-2-yl)carbamate (8f)

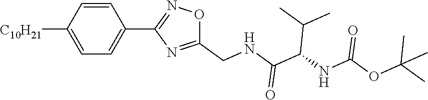

Synthesized according to General Procedure 8. Purified via column chromatography (30% ethyl acetate/hexanes). Yellow oil (80%, 47 mg). ¹H NMR (400 MHz, CDCl₃) δ 7.94 (d, J=8.4 Hz, 2H), 7.27 (d, J=7.9 Hz, 2H), 7.00 (t, J=5.8 Hz, 1H), 5.18-5.04 (m, 1H), 4.75 (qd, J=17.4, 5.8 Hz, 2H), 4.10-4.02 (m, 1H), 2.65 (t, J=7.8 Hz, 2H), 2.30-2.15 (m, 1H), 1.63 (p, J=7.3 Hz, 2H), 1.44 (s, 9H), 1.34-1.22 (m, 14H), 1.02 (d, J=6.7 Hz, 3H), 0.98 (d, J=6.8 Hz, 3H), 0.88 (t, J=6.8 Hz, 3H). ¹³C NMR (101 MHz, CDCl₃) δ 175.81, 172.50, 168.48, 156.19, 146.77, 129.00, 127.48, 123.88, 80.21, 59.97, 36.07, 35.77, 32.01, 31.31, 31.08, 29.72, 29.69, 29.59, 29.43, 29.39, 28.40, 22.79, 19.34, 18.10, 14.22. HRMS: (ESI) [M+H]+ calc. for C₂₉H₄₇N₄O₄, 515.3592, observed, 515.3594.

### tert-butyl (5-(((3-(4-decylphenyl)-1,2,4-oxadiazol-5-yl)methyl)amino)-5-oxopentyl)carbamate (8g)

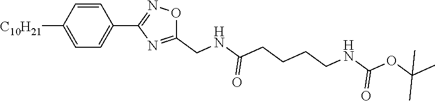

Synthesized according to General Procedure 8. Purified via column chromatography (40% ethyl acetate/hexanes). Yellow oil (96%, 141 mg). ¹H NMR (400 MHz, CDCl₃) δ 7.94 (d, J=8.1 Hz, 2H), 7.27 (d, J=8.1 Hz, 2H), 6.72 (brs, 1H), 4.78-4.70 (m, 3H), 3.15 (q, J=6.7 Hz, 2H), 2.65 (t, J=7.6 Hz, 2H), 2.35 (t, J=7.4 Hz, 2H), 1.72 (p, J=7.2 Hz, 2H), 1.66-1.51 (m, 4H), 1.41 (s, 9H), 1.36-1.22 (m, 14H), 0.88 (t, J=7.1 Hz, 3H). ¹³C NMR (101 MHz, CDCl₃) δ 176.07, 173.36, 168.48, 156.39, 146.85, 129.05, 127.50, 123.85, 79.35, 39.76, 36.08, 35.89, 35.48, 32.01, 31.32, 29.72, 29.69, 29.59, 29.48, 29.44, 29.39, 28.51, 22.80, 22.62, 14.23.

### tert-butyl (2-((2-(3-(4-decylphenyl)-1,2,4-oxadiazol-5-yl)ethyl)amino)-2-oxoethyl)carbamate (8h)

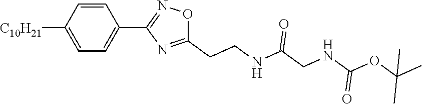

Synthesized according to General Procedure 8. Purified via column chromatography (35% ethyl acetate/dichloromethane). Clear oil (97%, 135 mg). ¹H NMR (400 MHz, cd₃od) δ 7.94 (d, J=8.1 Hz, 2H), 7.30 (d, J=8.1 Hz, 2H), 3.75-3.65 (m, 4H), 3.16 (t, J=6.6 Hz, 2H), 2.65 (t, J=7.7 Hz, 2H), 1.63 (p, J=7.3 Hz, 2H), 1.38 (s, 9H), 1.29 (d, J=22.5 Hz, 14H), 0.88 (t, J=6.7 Hz, 3H). ¹³C NMR (101 MHz, cd₃od) δ 179.42, 172.82, 169.38, 158.32, 147.85, 130.04, 128.37, 125.44, 80.69, 44.66, 37.25, 36.84, 33.07, 32.45, 30.73, 30.71, 30.59, 30.47, 30.30, 28.64, 27.55, 23.74, 14.48.

### tert-butyl (S)-2-(((3-(4-decylphenyl)-1,2,4-oxadiazol-5-yl)methyl)carbamoyl)piperidine-1-carboxylate (8i)

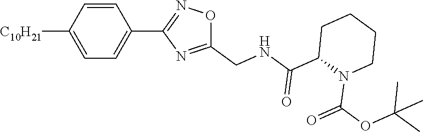

Synthesized according to General Procedure 8. Purified by silica chromatography (65% ethyl acetate in hexanes). Yellow oil (100%, 150 mg). ¹H NMR (400 MHz, Chloroform-d) δ 7.95 (d, J=8.3 Hz, 2H), 7.27 (d, J=8.2 Hz, 2H), 7.04 (brs, 1H), 4.98-4.87 (m, 2H), 4.61 (brs, 1H), 4.11 (brs, 1H), 2.95 (t, J=12.8 Hz, 1H), 2.65 (t, J=7.7 Hz, 3H), 2.34 (brs, 1H), 1.70-1.55 (m, 6H), 1.49 (s, 9H), 1.37-1.19 (m, 14H), 0.88 (t, J=6.7 Hz, 3H). ¹³C NMR (101 MHz, Chloroform-d) δ 175.97, 171.91, 168.47, 146.84, 129.03, 127.49, 123.87, 81.00, 53.93, 42.44, 36.08, 35.93, 32.01, 31.32, 29.72, 29.68, 29.59, 29.44, 29.39, 28.49, 25.45, 25.02, 22.80, 20.60, 14.23.

### tert-butyl (R)-3-(((3-(4-decylphenyl)-1,2,4-oxadiazol-5-yl)methyl)carbamoyl)pyrrolidine-1-carboxylate (8j)

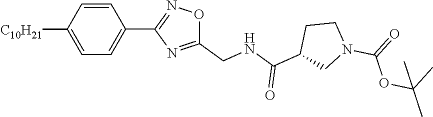

Synthesized according to General Procedure 8. Purified via column chromatography (35% ethyl acetate/hexanes). White solid (100%, 219 mg). ¹H NMR (400 MHz, CDCl₃) δ 7.88 (d, J=8.0 Hz, 2H), 7.41-7.16 (m, 3H), 4.81-4.55 (m, 2H), 3.71-3.40 (m, 3H), 3.29 (q, J=8.7 Hz, 1H), 3.00 (q, J=7.8 Hz, 1H), 2.60 (t, J=7.7 Hz, 2H), 2.20-2.04 (m, 2H), 1.57 (p, J=7.2 Hz, 2H), 1.40 (s, 9H), 1.33-1.16 (m, 14H), 0.84 (t, J=6.7 Hz, 3H). ¹³C NMR (101 MHz, CDCl₃) δ 175.88, 172.95, 168.40, 154.45, 146.77, 128.95, 127.37, 123.72, 79.57, 48.54, 45.54 (d, J=21.0 Hz), 43.91 (d, J=98.7 Hz), 38.60, 35.97, 35.86, 31.92, 31.22, 29.63, 29.60, 29.50, 29.35, 29.31, 28.49, 22.70, 14.14. HRMS: (ESI) [M+Na]+ calc. for C₂₉H₄₄N₄NaO₄, 535.3255, observed, 535.3253.

### tert-butyl (S)-3-(2-(((3-(4-decylphenyl)-1,2,4-oxadiazol-5-yl)methyl)amino)-2-oxoethyl)pyrrolidine-1-carboxylate (8k)

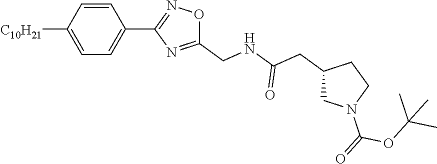

Synthesized according to General Procedure 8. Purified via column chromatography (60% ethyl acetate/hexanes). Yellow oil (98%, 221 mg). ¹H NMR (400 MHz, CDCl₃) δ 7.92 (d, J=7.9 Hz, 2H), 7.25 (d, J=8.3 Hz, 2H), 6.77 (dt, J=16.6, 10.7 Hz, 1H), 4.81-4.61 (m, 2H), 3.58 (q, J=9.4 Hz, 1H), 3.49-3.36 (m, 1H), 3.28 (h, J=8.8 Hz, 1H), 2.96 (t, J=9.6 Hz, 1H), 2.71-2.56 (m, 3H), 2.46-2.27 (m, 2H), 2.14-2.01 (m, 1H), 1.68-1.53 (m, 3H), 1.42 (s, 9H), 1.35-1.20 (m, 14H), 0.86 (t, J=6.6 Hz, 3H).

### tert-butyl (2-(((3-(4-decylphenyl)-1,2,4-oxadiazol-5-yl)methyl)amino)-2-oxoethyl)(methyl)carbamate (8l)

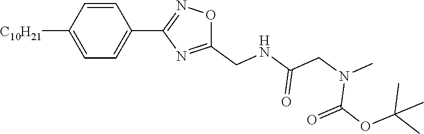

Synthesized according to General Procedure 8. Purified via column chromatography (35% ethyl acetate/hexanes). White solid (86%, 83 mg). ¹H NMR (400 MHz, CDCl₃) δ 7.95 (d, J=8.3 Hz, 2H), 7.27 (d, J=8.3 Hz, 2H), 6.89 (brs, 1H), 4.77 (d, J=5.7 Hz, 2H), 3.97 (s, 2H), 3.00 (s, 3H), 2.65 (t, J=7.6 Hz, 2H), 1.63 (p, J=7.3 Hz, 2H), 1.48 (s, 9H), 1.38-1.21 (m, 14H), 0.87 (t, J=6.8 Hz, 3H). ¹³C NMR (101 MHz, CDCl₃) δ 175.60, 168.54, 146.92, 129.06, 127.54, 123.80, 81.27, 53.27, 36.11, 36.01, 35.78, 32.03, 31.35, 29.74, 29.71, 29.61, 29.46, 29.41, 28.45, 22.82, 14.26. HRMS: (ESI) [M+H]+ calc. for C₂₇H₄₃N₄O₄, 487.3279, observed, 487.3277.

### tert-butyl (S)-2-(((3-(4-decylphenyl)-1,2,4-oxadiazol-5-yl)methyl)carbamoyl)pyrrolidine-1-carboxylate (8m)

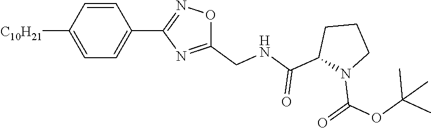

Synthesized according to General Procedure 8. Purified by silica chromatography (35% ethyl acetate in hexanes). Yellow oil (95%, 278 mg). ¹H NMR (400 MHz, Chloroform-d) δ 7.95 (d, J=8.2 Hz, 2H), 7.27 (d, J=8.2 Hz, 2H), 4.87-4.60 (m, 2H), 4.41 (s, 1H), 3.43 (d, J=47.0 Hz, 2H), 2.68-2.60 (m, 2H), 2.49-2.13 (m, 1H), 2.02-1.85 (m, 3H), 1.68-1.58 (m, 2H), 1.47 (s, 9H), 1.38-1.20 (m, 14H), 0.90-0.85 (m, 3H). ¹³C NMR (101 MHz, Chloroform-d) δ 175.95, 172.59, 168.49, 156.28, 146.76, 128.98, 127.50, 123.88, 80.89, 60.72, 47.26, 36.06, 35.83, 31.99, 31.30, 29.70, 29.67, 29.57, 29.50, 29.42, 29.36, 28.47, 24.44, 22.78, 14.21.

### tert-butyl ((2S,3S)-1-(((3-(4-decylphenyl)-1,2,4-oxadiazol-5-yl)methyl)amino)-3-methyl-1-oxopentan-2-yl)carbamate (8n)

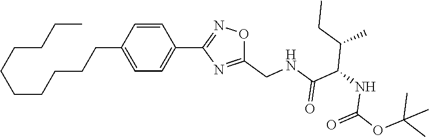 [SMILES: CCCCCCCCCCc1ccc(-c2noc(CNC(=O)[C@@H](NC(=O)OC(C)(C)C)[C@@H](C)CC)n2)cc1]

Synthesized according to General Procedure 8. Purified via column chromatography (30% ethyl acetate/hexanes). Yellow oil (92%, 138 mg). ¹H NMR (400 MHz, CDCl₃) δ 7.93 (d, J=8.2 Hz, 2H), 7.34-7.22 (m, 3H), 5.22 (d, J=8.9 Hz, 1H), 4.89-4.61 (m, 2H), 4.12 (t, J=8.1 Hz, 1H), 2.64 (t, J=7.7 Hz, 2H), 1.98-1.89 (m, 1H), 1.69-1.54 (m, 3H), 1.42 (s, 9H), 1.37-1.22 (m, 14H), 0.99 (d, J=6.8 Hz, 3H), 0.96-0.80 (m, 6H). ¹³C NMR (101 MHz, CDCl₃) δ 175.78, 172.46, 168.48, 156.12, 146.80, 129.01, 127.51, 123.89, 80.27, 59.30, 37.31, 36.09, 35.80, 32.02, 31.33, 29.73, 29.70, 29.60, 29.45, 29.40, 28.41, 24.88, 22.81, 15.64, 14.24, 11.49.

### tert-butyl (S)-3-(2-(((3-(4-decylphenyl)-1,2,4-oxadiazol-5-yl)methyl)amino)-2-oxoethyl)piperidine-1-carboxylate (8o)

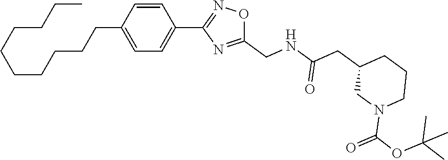 [SMILES: CCCCCCCCCCc1ccc(-c2noc(CNC(=O)C[C@@H]3CCCN(C(=O)OC(C)(C)C)C3)n2)cc1]

Synthesized according to General Procedure 8. Purified via column chromatography (40% ethyl acetate/hexanes). Yellow oil (92%, 141 mg). ¹H NMR (400 MHz, CDCl₃) δ 7.94 (d, J=8.1 Hz, 2H), 7.45 (brs, 1H), 7.26 (d, J=8.1 Hz, 2H), 4.73 (d, J=5.7 Hz, 2H), 3.94-2.80 (m, 6H), 2.64 (t, J=7.7 Hz, 2H), 2.40-2.26 (m, 1H), 2.16-2.08 (m, 2H), 1.96-1.82 (m, 1H), 1.66-1.54 (m, 3H), 1.44 (s, 9H), 1.38-1.18 (m, 14H), 0.87 (t, J=6.8 Hz, 3H). ¹³C NMR (101 MHz, CDCl₃) δ 176.10, 172.33, 168.48, 146.79, 129.02, 127.51, 123.93, 79.87, 48.13, 45.09, 39.44, 36.08, 36.01, 33.22, 32.01, 31.32, 30.75, 29.72, 29.69, 29.59, 29.44, 29.39, 28.54, 23.56, 22.80, 14.23.

### tert-butyl 4-(((3-(4-decylphenyl)-1,2,4-oxadiazol-5-yl)methyl)carbamoyl)piperidine-1-carboxylate (8p)

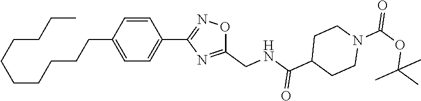 [SMILES: CCCCCCCCCCc1ccc(-c2noc(CNC(=O)C3CCN(C(=O)OC(C)(C)C)CC3)n2)cc1]

Synthesized according to General Procedure 8. Purified via column chromatography (60% ethyl acetate/hexanes). Clear oil (100%, 150 mg). ¹H NMR (400 MHz, CDCl₃) δ 7.95 (d, J=8.1 Hz, 2H), 7.28 (d, J=8.1 Hz, 2H), 6.37 (t, J=5.6 Hz, 1H), 4.74 (d, J=5.5 Hz, 2H), 4.30-4.03 (m, 2H), 2.79 (t, J=12.7 Hz, 2H), 2.65 (t, J=7.7 Hz, 2H), 2.39 (tt, J=11.4, 3.7 Hz, 1H), 1.92-1.84 (m, 2H), 1.81-1.57 (m, 4H), 1.46 (s, 9H), 1.39-1.20 (m, 14H), 0.88 (t, J=6.7 Hz, 3H). ¹³C NMR (101 MHz, CDCl₃) δ 175.83, 174.68, 168.50, 154.78, 146.96, 129.09, 127.52, 123.75, 79.84, 43.11, 36.10, 35.96, 32.02, 31.33, 29.73, 29.70, 29.60, 29.45, 29.40, 29.39, 28.59, 28.56, 22.81, 14.24.

### tert-butyl (R)-2-(((3-(4-decylphenyl)-1,2,4-oxadiazol-5-yl)methyl)carbamoyl)morpholine-4-carboxylate (8q)

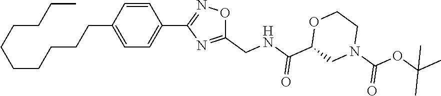 [SMILES: CCCCCCCCCCc1ccc(-c2noc(CNC(=O)[C@H]3CN(C(=O)OC(C)(C)C)CCO3)n2)cc1]

Synthesized according to General Procedure 8. Purified via column chromatography (65% ethyl acetate/hexanes). Clear oil that turns to white solid (40%, 60 mg). ¹H NMR (400 MHz, CDCl₃) δ 7.96 (d, J=8.2 Hz, 2H), 7.29 (d, J=8.0 Hz, 3H), 4.78 (d, J=5.8 Hz, 2H), 4.36 (d, J=12.2 Hz, 1H), 4.06-3.90 (m, 3H), 3.62 (td, J=11.7, 2.8 Hz, 1H), 2.89 (dt, J=40.4, 12.0 Hz, 2H), 2.66 (t, J=7.7 Hz, 2H), 1.63 (p, J=7.8 Hz, 2H), 1.47 (s, 9H), 1.38-1.20 (m, 14H), 0.88 (t, J=6.7 Hz, 3H). ¹³C NMR (101 MHz, CDCl₃) δ 175.37, 169.37, 168.63, 154.65, 146.93, 129.08, 127.56, 123.78, 80.74, 75.06, 66.70, 46.13, 42.88, 36.10, 35.29, 32.02, 31.33, 29.73, 29.70, 29.60, 29.45, 29.38, 28.49, 22.81, 14.25.

### tert-butyl ((1S,3R)-3-(((3-(4-decylphenyl)-1,2,4-oxadiazol-5-yl)methyl)carbamoyl)cyclohexyl)carbamate (8r)

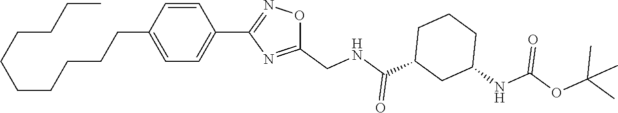 [SMILES: CCCCCCCCCCc1ccc(-c2noc(CNC(=O)[C@@H]3CCC[C@H](NC(=O)OC(C)(C)C)C3)n2)cc1]

Synthesized according to General Procedure 8. Purified via column chromatography (35% ethyl acetate/hexanes). Yellow oil (93%, 143 mg). ¹H NMR (400 MHz, CDCl₃) δ 7.94 (d, J=8.0 Hz, 2H), 7.26 (d, J=8.1 Hz, 2H), 6.78 (brs, 1H), 4.78-4.62 (m, 3H), 3.55-3.45 (m, 1H), 2.64 (t, J=15.5 Hz, 2H), 2.35 (tt, J=10.2, 5.4 Hz, 1H), 2.24-2.14 (m, 1H), 2.00-1.91 (m, 1H), 1.91-1.82 (m, 2H), 1.63 (p, J=7.3 Hz, 2H), 1.50-1.20 (m, 26H), 1.16-1.04 (m, 1H), 0.87 (t, J=7.0 Hz, 3H). ¹³C NMR (101 MHz, CDCl₃) δ 176.02, 175.34, 168.47, 155.35, 146.81, 129.01, 127.50, 123.83, 79.38, 49.15, 44.12, 36.20, 36.06, 35.86, 32.75, 31.99, 31.30, 29.70, 29.67, 29.57, 29.42, 29.38, 28.57, 28.52, 24.39, 22.78, 14.22. HRMS: (ESI) [M+H]+ calc. for C₃₁H₄₉N₄O₄, 541.3748, observed, 541.3743.

### tert-butyl (S)-(1-(((3-(4-decylphenyl)-1,2,4-oxadiazol-5-yl)methyl)amino)-3-hydroxy-1-oxopropan-2-yl)carbamate (8s)

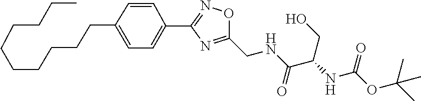 [SMILES: CCCCCCCCCCc1ccc(-c2noc(CNC(=O)[C@H](CO)NC(=O)OC(C)(C)C)n2)cc1]

Synthesized according to General Procedure 8. Purified via column chromatography (80-100% ethyl acetate/hexanes). Yellow oil (99%, 143 mg). ¹H NMR (400 MHz, CDCl₃) δ 7.89 (d, J=7.8 Hz, 2H), 7.67 (t, J=6.0 Hz, 1H), 7.26 (d, J=8.1 Hz, 2H), 5.73 (d, J=7.4 Hz, 1H), 4.83-4.65 (m, 2H), 4.44-4.29 (m, 1H), 4.12 (dd, J=11.4, 3.4 Hz, 1H), 4.04-3.86 (m, 1H), 3.82-3.66 (m, 1H), 2.64 (t, J=7.8 Hz, 2H), 1.62 (p, J=7.0 Hz, 2H), 1.44 (s, 9H), 1.38-1.20 (m, 14H), 0.88 (t, J=6.7 Hz, 3H). ¹³C NMR (101 MHz, CDCl₃) δ 176.26, 172.26, 168.20, 156.17, 147.03, 129.12, 127.45, 123.46, 80.82, 63.26, 55.66, 36.08, 36.04, 32.02, 31.31, 29.73, 29.70, 29.60, 29.45, 29.40, 28.41, 22.81, 14.25. HRMS: (ESI) [M+H]+ calc. for C₂₇H₄₃N₄O₅, 503.3228, observed, 503.3228.

### tert-butyl (R)-2-(((3-(4-decylphenyl)-1,2,4-oxadiazol-5-yl)methyl)carbamoyl)piperidine-1-carboxylate (8t)

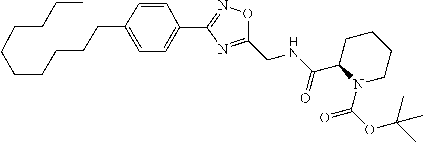 [SMILES: CCCCCCCCCCc1ccc(-c2noc(CNC(=O)[C@H]3CCCCN3C(=O)OC(C)(C)C)n2)cc1]

Synthesized according to General Procedure 8. Purified via column chromatography (30% ethyl acetate/hexanes). Yellow oil (93%, 139 mg). ¹H NMR (400 MHz, CDCl₃) δ 7.95 (d, J=8.2 Hz, 2H), 7.27 (d, J=8.1 Hz, 2H), 7.01 (brs, 1H), 4.97-4.79 (m, 2H), 4.70-4.51 (m, 1H), 4.22-3.99 (m, 1H), 2.96 (t, J=12.9 Hz, 1H), 2.65 (t, J=7.7 Hz, 2H), 2.39-2.26 (m, 1H), 1.72-1.53 (m, 7H), 1.49 (s, 9H), 1.36-1.21 (m, 14H), 0.88 (t, J=6.7 Hz, 3H). ¹³C NMR (101 MHz, CDCl₃) δ 175.97, 171.91, 168.45, 146.82, 129.01, 127.48, 123.87, 80.97, 53.77, 42.50, 36.07, 35.92, 32.00, 31.31, 29.71, 29.67, 29.58, 29.43, 29.37, 28.47, 25.47, 25.01, 22.79, 20.59, 14.22. HRMS: (ESI) [M+Na]+ calc. for C₃₀H₄₆N₄NaO₄, 549.3411, observed, 549.3411.

### tert-butyl 2-(((3-(4-decylphenyl)-1,2,4-oxadiazol-5-yl)methyl)carbamoyl)azepane-1-carboxylate (8u)

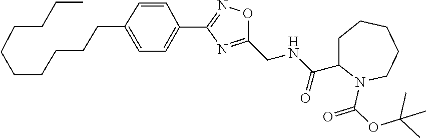 [SMILES: CCCCCCCCCCc1ccc(-c2noc(CNC(=O)C3CCCCCN3C(=O)OC(C)(C)C)n2)cc1]

Synthesized according to General Procedure 8. Purified via column chromatography (20% ethyl acetate/hexanes). Yellow oil (94%, 144 mg). ¹H NMR (400 MHz, CDCl₃) δ 7.94 (d, J=8.3 Hz, 2H), 7.34 (t, J=5.8 Hz, 1H), 7.26 (d, J=8.2 Hz, 2H), 4.84-4.52 (m, 3H), 4.00-3.70 (m, 1H), 3.04-2.85 (m, 1H), 2.64 (t, J=7.8 Hz, 2H), 2.40-2.09 (m, 1H), 2.00-1.56 (m, 7H), 1.51-1.41 (m, 11H), 1.37-1.19 (m, 14H), 0.87 (t, J=6.8 Hz, 3H). ¹³C NMR (101 MHz, CDCl₃) δ 175.97, 172.85, 168.46, 157.20, 146.71, 128.97, 127.50, 123.97, 80.68, 57.96, 43.91, 36.06, 35.74, 31.99, 31.30, 29.70, 29.67, 29.57, 29.45, 29.42, 29.36, 28.89, 28.52, 28.49, 24.74, 22.77, 14.21. HRMS: (ESI) [M+H]+ calc. for C₃₁H₄₉N₄O₄, 541.3748, observed, 541.3735.

### tert-butyl (2S,4S)-2-(((3-(4-decylphenyl)-1,2,4-oxadiazol-5-yl)methyl)carbamoyl)-4-hydroxypyrrolidine-1-carboxylate (8v)

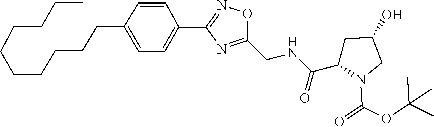 [SMILES: CCCCCCCCCCc1ccc(-c2noc(CNC(=O)[C@@H]3C[C@H](O)CN3C(=O)OC(C)(C)C)n2)cc1]

Synthesized according to General Procedure 8. Purified via column chromatography (80-100% ethyl acetate/hexanes). White solid (93%, 84 mg). ¹H NMR (400 MHz, CDCl₃) δ 8.10 (brs, 1H), 7.94 (d, J=7.9 Hz, 2H), 7.27 (d, J=7.8 Hz, 2H), 4.87-4.66 (m, 3H), 4.59-4.28 (m, 2H), 3.75-3.42 (m, 2H), 2.65 (t, J=7.7 Hz, 2H), 2.44-2.14 (m, 2H), 1.62 (p, J=7.4 Hz, 2H), 1.46 (s, 9H), 1.37-1.21 (m, 14H), 0.88 (t, J=6.7 Hz, 3H). ¹³C NMR (101 MHz, CDCl₃) δ 175.52, 174.17, 168.44, 155.82, 146.78, 128.98, 127.49, 123.80, 81.09, 70.86, 59.43, 56.93, 38.68, 36.57, 36.04, 31.97, 31.28, 29.68, 29.65, 29.56, 29.40, 29.35, 28.43, 22.76, 14.20. HRMS: (ESI) [M+H]+ calc. for C₂₉H₄₅N₄O₅, 529.3384, observed, 529.3374.

### tert-butyl (2S,4R)-2-(((3-(4-decylphenyl)-1,2,4-oxadiazol-5-yl)methyl)carbamoyl)-4-hydroxypyrrolidine-1-carboxylate (8w)

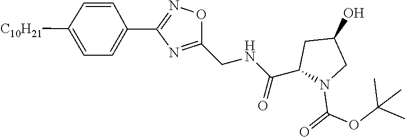

Synthesized according to General Procedure 8. Purified via column chromatography (80-100% ethyl acetate/hexanes). Yellow oil (96%, 144 mg). ¹H NMR (400 MHz, CDCl₃) δ 7.92 (d, J=7.9 Hz, 2H), 7.26 (d, J=8.0 Hz, 2H), 4.72 (d, J=5.8 Hz, 2H), 4.59-4.41 (m, 2H), 3.75-3.12 (m, 3H), 2.64 (t, J=7.7 Hz, 2H), 2.54-2.08 (m, 2H), 1.62 (p, J=7.2 Hz, 2H), 1.44 (s, 9H), 1.36-1.22 (m, 14H), 0.88 (t, J=6.7 Hz, 3H). ¹³C NMR (101 MHz, CDCl₃) δ 175.76, 172.45, 168.50, 156.23, 146.84, 129.00, 127.47, 123.76, 81.20, 69.79, 58.52, 54.87, 38.71, 36.05, 35.76, 31.99, 31.29, 29.70, 29.67, 29.57, 29.42, 29.38, 28.40, 22.77, 14.21. HRMS: (ESI) [M+H]+ calc. for C₂₉H₄₅N₄O₅, 529.3384, observed, 529.3380.

### tert-butyl (S)-3-(((3-(4-decylphenyl)-1,2,4-oxadiazol-5-yl)methyl)carbamoyl)pyrrolidine-1-carboxylate (8x)

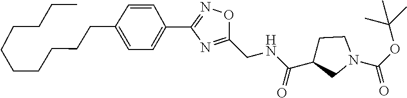 [SMILES: CCCCCCCCCCc1ccc(-c2noc(CNC(=O)[C@H]3CCN(C(=O)OC(C)(C)C)C3)n2)cc1]

Synthesized according to General Procedure 8. Purified via column chromatography (35% ethyl acetate/hexanes). White solid (100%, 220 mg). ¹H NMR (400 MHz, CDCl₃) δ 7.93 (d, J=8.0 Hz, 2H), 7.26 (d, J=8.0 Hz, 2H), 7.07 (brs, 1H), 4.83-4.61 (m, 2H), 3.75-3.45 (m, 3H), 3.34 (q, J=8.5 Hz, 1H), 3.01 (q, J=7.3 Hz, 1H), 2.64 (t, J=7.7 Hz, 2H), 2.28-2.07 (m, 2H), 1.62 (p, J=7.7 Hz, 2H), 1.44 (s, 9H), 1.37-1.21 (m, 14H), 0.88 (t, J=6.7 Hz, 3H). ¹³C NMR (101 MHz, CDCl₃) δ 175.84, 172.90, 168.46, 154.47, 146.86, 129.01, 127.44, 123.74, 79.63, 48.60, 45.63 (d, J=13.6 Hz), 44.06 (d, J=100.0 Hz), 38.67, 36.03, 35.94, 31.97, 31.27, 29.68, 29.65, 29.55, 29.40, 29.35, 28.55, 22.75, 14.19. HRMS: (ESI) [M+Na]+ calc. for C₂₉H₄₄N₄NaO₄, 535.3255, observed, 535.3247.

### tert-butyl (R)-(4-(((3-(4-decylphenyl)-1,2,4-oxadiazol-5-yl)methyl)amino)-2-hydroxy-4-oxobutyl)carbamate (8y)

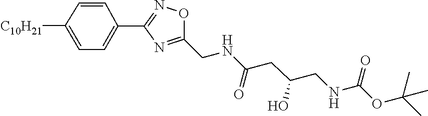

Synthesized according to General Procedure 8. Purified via column chromatography (80% ethyl acetate/hexanes). White solid (94%, 32 mg). ¹H NMR (400 MHz, CDCl₃) δ 11.43 (s, 1H), 8.33 (t, J=5.4 Hz, 1H), 7.52 (d, J=1.8 Hz, 1H), 7.31-7.20 (m, 4H), 6.24 (d, J=1.8 Hz, 1H), 4.19 (t, J=7.1 Hz, 2H), 3.38 (q, J=6.9 Hz, 2H), 2.63 (t, J=7.9 Hz, 2H), 2.11 (p, J=7.0 Hz, 2H), 1.65 (p, J=7.6 Hz, 2H), 1.49 (s, 18H), 1.42-1.21 (m, 14H), 0.88 (t, J=6.6 Hz, 3H). ¹³C NMR (101 MHz, CDCl₃) δ 163.66, 156.27, 153.22, 143.82, 143.61, 139.00, 128.88, 128.87, 128.02, 106.18, 83.13, 79.34, 47.00, 38.30, 35.90, 32.05, 31.49, 29.87, 29.78, 29.73, 29.65, 29.55, 29.48, 28.44, 28.21, 22.83, 14.26.

### tert-butyl (S)-2-(((3-(4-decylphenyl)-1,2,4-oxadiazol-5-yl)methyl)carbamoyl)azetidine-1-carboxylate (8z)

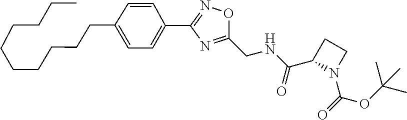 [SMILES: CCCCCCCCCCc1ccc(-c2noc(CNC(=O)[C@@H]3CCN3C(=O)OC(C)(C)C)n2)cc1]

Synthesized according to General Procedure 8. Purified via column chromatography (70% ethyl acetate/hexanes). Yellow solid (100%, 142 mg). ¹H NMR (400 MHz, CDCl₃) δ 8.48-7.80 (m, 3H), 7.27 (d, J=8.1 Hz, 2H), 4.89-4.68 (m, 3H), 3.95 (q, J=8.3 Hz, 1H), 3.89-3.79 (m, 1H), 2.65 (t, J=7.7 Hz, 2H), 2.56-2.43 (m, 2H), 1.63 (q, J=7.5 Hz, 2H), 1.48 (s, 9H), 1.36-1.22 (m, 14H), 0.88 (t, J=6.8 Hz, 3H). ¹³C NMR (101 MHz, CDCl₃) δ 175.65, 172.12, 168.53, 157.57, 146.76, 128.97, 127.52, 123.87, 81.35, 62.30, 47.41, 36.06, 35.71, 31.99, 31.32, 29.70, 29.67, 29.57, 29.42, 29.37, 28.39, 22.78, 19.64, 14.23. HRMS: (ESI) [M+H]+ calc. for C₂₈H₄₃N₄O₄, 499.3279, observed, 499.3277.

### tert-butyl (S)-5-((tert-butoxycarbonyl)amino)-6-(((3-(4-decylphenyl)-1,2,4-oxadiazol-5-yl)methyl)amino)-6-oxohexanoate (8aa)

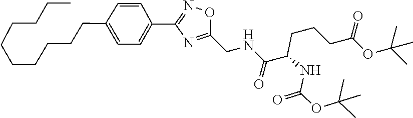 [SMILES: CCCCCCCCCCc1ccc(-c2noc(CNC(=O)[C@H](CCCC(=O)OC(C)(C)C)NC(=O)OC(C)(C)C)n2)cc1]

Synthesized according to General Procedure 8. Purification via column chromatography (30-40% ethyl acetate/hexanes). Yellow oil (30%, 67 mg). ¹H NMR (400 MHz, CDCl₃) δ 7.95 (d, J=8.3 Hz, 2H), 7.36 (t, J=6.1 Hz, 1H), 7.27 (d, J=8.0 Hz, 2H), 5.23 (d, J=7.8 Hz, 1H), 4.75 (d, J=5.6 Hz, 2H), 4.23 (s, 1H), 2.65 (t, J=7.7 Hz, 2H), 2.34-2.22 (m, 2H), 1.80-1.56 (m, 6H), 1.53-1.40 (m, 18H), 1.37-1.26 (m, 14H), 0.88 (t, J=6.8 Hz, 3H). ¹³C NMR (101 MHz, CDCl₃) δ 175.71, 172.90, 172.54, 168.51, 156.04, 146.81, 129.02, 127.54, 123.88, 80.63, 80.52, 54.20, 45.58, 36.09, 35.89, 34.91, 32.02, 31.33, 29.73, 29.70, 29.60, 29.45, 29.40, 28.43, 28.23, 22.81, 21.06, 14.24. HRMS: (ESI) [M+H]+ calc. for C₃₄H₅₅N₄O₆, 615.4116, observed, 615.4113.

### N-((3-(4-decylphenyl)-1,2,4-oxadiazol-5-yl)methyl)-2-(dimethylamino)acetamide (8ab)

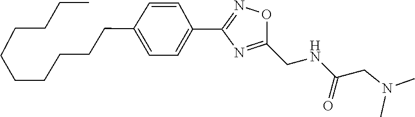 [SMILES: CCCCCCCCCCc1ccc(-c2noc(CNC(=O)CN(C)C)n2)cc1]

Synthesized according to General Procedure 8. Purified via column chromatography (100% ethyl acetate). White solid (88%, 77 mg). ¹H NMR (400 MHz, cd₃od) δ 7.93 (d, J=8.4 Hz, 2H), 7.31 (d, J=8.4 Hz, 2H), 4.72 (s, 2H), 3.09 (s, 2H), 2.67 (t, J=7.7 Hz, 2H), 2.36 (s, 6H), 1.63 (p, J=7.4 Hz, 2H), 1.40-1.22 (m, 14H), 0.90 (t, J=6.9 Hz, 3H). ¹³C NMR (101 MHz, cd₃od) δ 178.27, 173.81, 169.57, 148.05, 130.09, 128.32, 125.30, 63.43, 46.02, 36.84, 36.26, 33.06, 32.42, 30.70, 30.69, 30.55, 30.44, 30.30, 23.72, 14.44.

### tert-butyl (S)-(1-(((3-(4-decylphenyl)-1,2,4-oxadiazol-5-yl)methyl)amino)-1-oxo-3-phenylpropan-2-yl)carbamate (8ac)

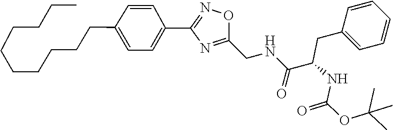 [SMILES: CCCCCCCCCCc1ccc(-c2noc(CNC(=O)[C@H](Cc3ccccc3)NC(=O)OC(C)(C)C)n2)cc1]

Synthesized according to General Procedure 8. Purified via column chromatography (30% ethyl acetate). Yellow oil (98%, 157 mg). ¹H NMR (400 MHz, CDCl₃) δ 7.92 (d, J=8.1 Hz, 2H), 7.28-7.13 (m, 7H), 6.92 (brs, 1H), 5.16 (d, J=7.9 Hz, 1H), 4.65 (d, J=5.7 Hz, 2H), 4.56-4.42 (m, 1H), 3.15-3.03 (m, 2H), 2.63 (t, J=7.7 Hz, 2H), 1.62 (p, J=7.4 Hz, 2H), 1.44-1.19 (m, 23H), 0.86 (t, J=6.8 Hz, 3H). ¹³C NMR (101 MHz, CDCl₃) δ 175.44, 171.92, 168.48, 155.72, 146.86, 136.49, 129.41, 129.03, 128.81, 127.53, 127.13, 123.80, 80.58, 55.81, 38.44, 36.09, 35.74, 32.02, 31.34, 29.73, 29.70, 29.60, 29.45, 29.40, 28.37, 22.81, 14.25.

### tert-butyl (S)-(1-(((3-(4-decylphenyl)-1,2,4-oxadiazol-5-yl)methyl)amino)-4-methyl-1-oxopentan-2-yl)carbamate (8ad)

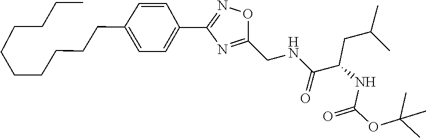 [SMILES: CCCCCCCCCCc1ccc(-c2noc(CNC(=O)[C@H](CC(C)C)NC(=O)OC(C)(C)C)n2)cc1]

Synthesized according to General Procedure 8. Purified via column chromatography (30% ethyl acetate). Yellow oil (98%, 147 mg). ¹H NMR (400 MHz, CDCl₃) δ 7.94 (d, J=8.3 Hz, 2H), 7.33-7.22 (m, 3H), 5.06 (d, J=8.3 Hz, 1H), 4.82-4.66 (m, 2H), 4.26 (s, 1H), 2.65 (t, J=7.7 Hz, 2H), 1.79-1.70 (m, 2H), 1.64 (p, J=7.7 Hz, 2H), 1.54 (dd, J=9.3, 8.0 Hz, 1H), 1.43 (s, 9H), 1.38-1.19 (m, 14H), 0.96 (dd, J=7.5, 6.2 Hz, 6H), 0.88 (t, J=6.8 Hz, 3H). ¹³C NMR (101 MHz, CDCl₃) δ 175.76, 173.24, 168.50, 156.06, 146.82, 129.01, 127.53, 123.88, 80.55, 53.04, 41.01, 36.10, 35.87, 32.02, 31.34, 29.73, 29.71, 29.60, 29.45, 29.41, 28.42, 24.84, 23.04, 22.81, 22.12, 14.24.

### tert-butyl (2S,4R)-2-(((3-(4-decylphenyl)-1,2,4-oxadiazol-5-yl)methyl)carbamoyl)-4-fluoropyrrolidine-1-carboxylate (8ae)

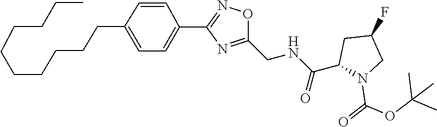 [SMILES: CCCCCCCCCCc1ccc(-c2noc(CNC(=O)[C@@H]3C[C@@H](F)CN3C(=O)OC(C)(C)C)n2)cc1]

Synthesized according to General Procedure 8. Purified via column chromatography (50% ethyl acetate). Yellow oil (95%, 143 mg). ¹H NMR (400 MHz, CDCl₃) δ 7.94 (d, J=8.0 Hz, 2H), 7.27 (d, J=8.1 Hz, 2H), 5.20 (dt, J=52.9, 3.9 Hz, 1H), 4.74 (d, J=5.6 Hz, 2H), 4.68-4.39 (m, 1H), 4.11-3.85 (m, 1H), 3.66-3.37 (m, 1H), 2.78-2.35 (m, 2H), 1.63 (p, J=7.1 Hz, 2H), 1.53-1.39 (m, 9H), 1.38-1.20 (m, 14H), 0.87 (t, J=7.0 Hz, 1H). ¹³C NMR (101 MHz, CDCl₃) δ 175.71, 171.61, 168.52, 156.04, 146.79, 129.01, 127.51, 123.88, 91.91 (d, J=177.1 Hz), 81.56, 58.07, 53.57 (d, J=23.1 Hz), 36.07, 34.65 (d, J=20.6 Hz), 32.00, 31.31, 29.71, 29.68, 29.58, 29.43, 29.38, 28.40, 22.79, 14.22.

### (S)-2-amino-N-((3-(4-decylphenyl)-1,2,4-oxadiazol-5-yl)methyl)propanamide hydrochloride (9a)

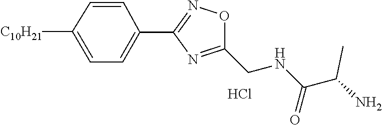

Synthesized according to General Procedure 6. Purified via trituration with ethyl acetate and diethyl ether. White solid (84%, 184 mg). ¹H NMR (400 MHz, cd₃od) δ 7.93 (d, J=8.3 Hz, 2H), 7.33 (d, J=8.2 Hz, 2H), 4.83-4.70 (m, 2H), 4.07 (q, J=7.1 Hz, 1H), 2.68 (t, J=7.7 Hz, 2H), 1.66 (p, J=7.4 Hz, 2H), 1.60 (d, J=7.1 Hz, 3H), 1.42-1.20 (m, 14H), 0.89 (t, J=6.8 Hz, 3H). ¹³C NMR (101 MHz, cd₃od) δ 177.82, 171.58, 169.62, 148.17, 130.11, 128.31, 125.18, 50.23, 36.84, 36.55, 33.06, 32.43, 30.69, 30.68, 30.55, 30.43, 30.30, 23.72, 17.47, 14.44. HRMS: (ESI) [M+H]+ calc. for C₂₂H₃₅N₄O₂, 387.2755, observed, 387.2764.

### 2-amino-N-((3-(4-decylphenyl)-1,2,4-oxadiazol-5-yl)methyl)acetamide hydrochloride (9b)

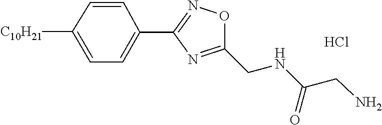

Synthesized according to General Procedure 6. White solid (69%, 159 mg). ¹H NMR (400 MHz, Methanol-d₄) δ 7.94 (d, J=8.3 Hz, 2H), 7.34 (d, J=8.1 Hz, 2H), 4.78 (s, 2H), 3.82 (s, 2H), 2.68 (t, J=7.7 Hz, 2H), 1.65 (p, J=7.3 Hz, 2H), 1.41-1.21 (m, 14H), 0.89 (t, J=6.6 Hz, 3H). ¹³C NMR (101 MHz, Methanol-d₄) δ 177.87, 169.62, 167.97, 148.22, 130.13, 128.32, 125.16, 41.49, 36.84, 36.46, 33.07, 32.46, 30.71, 30.69, 30.57, 30.45, 30.31, 23.74, 14.44.

### N-((3-(4-decylphenyl)-1,2,4-oxadiazol-5-yl)methyl)-2-(piperazin-1-yl)acetamide hydrochloride (9c)

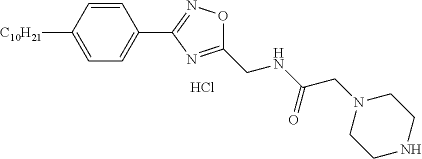

Synthesized according to General Procedure 6. White solid (76%, 125 mg). ¹H NMR (400 MHz, Methanol-d_{4) δ} 7.93 (d, J=8.2 Hz, 2H), 7.34 (d, J=8.2 Hz, 2H), 4.77 (s, 2H), 3.34-3.29 (m, 4H), 3.27 (s, 2H), 2.87 (t, J=7.8, 4H), 2.69 (t, J=7.7 Hz, 2H), 1.65 (p, J=7.3 Hz, 2H), 1.36-1.23 (m, 14H), 0.90 (t, J=6.8 Hz, 3H). ¹³C NMR (101 MHz, Methanol-d₄) δ 178.32, 172.97, 169.54, 148.10, 130.11, 128.28, 125.23, 61.61, 51.27, 44.91, 36.83, 36.36, 33.04, 32.42, 30.68, 30.67, 30.54, 30.42, 30.29, 23.71, 14.43. HRMS: (ESI) [M+H]+ calc. for C₂₅H₄₀N₅O₂, 442.3177, observed, 442.3171.

### 4-amino-N-((3-(4-decylphenyl)-1,2,4-oxadiazol-5-yl)methyl)butanamide hydrochloride (9d)

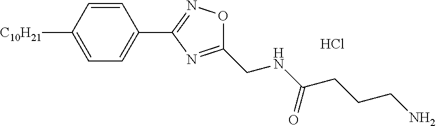

Synthesized according to General Procedure 6. White solid (76%, 108 mg). ¹H NMR (400 MHz, Methanol-d₄) δ 7.93 (d, J=7.9 Hz, 2H), 7.31 (d, J=7.9 Hz, 2H), 4.69 (s, 2H), 3.02 (t, J=7.5 Hz, 2H), 2.66 (t, J=7.7 Hz, 2H), 2.50 (t, J=7.0 Hz, 2H), 1.99 (p, J=7.3 Hz, 2H), 1.64 (p, J=7.1 Hz, 2H), 1.34-1.22 (m, 14H), 0.88 (t, J=6.6 Hz, 3H). ¹³C NMR (101 MHz, cd₃od) δ 176.82, 173.60, 168.14, 146.63, 128.67, 126.90, 123.81, 38.88, 35.43, 35.13, 31.86, 31.64, 31.01, 29.28, 29.15, 29.03, 28.90, 22.83, 22.31, 13.05.

### 3-amino-N-((3-(4-decylphenyl)-1,2,4-oxadiazol-5-yl)methyl)propanamide hydrochloride (9e)

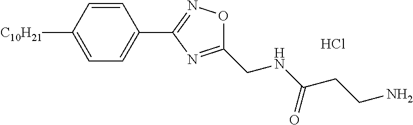

Synthesized according to General Procedure 6. Purified via trituration with ethyl acetate. White solid (87%, 171 mg). ¹H NMR (400 MHz, cd₃od) δ 7.95 (d, J=8.3 Hz, 2H), 7.34 (d, J=8.2 Hz, 2H), 4.74 (s, 2H), 3.25 (t, J=6.4 Hz, 2H), 2.77 (t, J=6.4 Hz, 2H), 2.69 (t, J=7.7 Hz, 2H), 1.66 (p, J=7.7 Hz, 2H), 1.40-1.24 (m, 14H), 0.90 (t, J=6.8 Hz, 3H). ¹³C NMR (101 MHz, cd₃od) δ 178.19, 172.69, 169.58, 148.14, 130.11, 128.32, 125.19, 36.87, 36.84, 36.38, 33.06, 32.59, 32.43, 30.70, 30.68, 30.55, 30.44, 30.30, 23.72, 14.44. HRMS: (ESI) [M+H]+ calc. for C₂₂H₃₅N₄O₂, 387.2755, observed, 387.2763.

### (S)-2-amino-N-((3-(4-decylphenyl)-1,2,4-oxadiazol-5-yl)methyl)-3-methylbutanamide hydrochloride (9f)

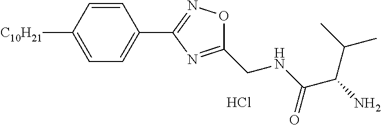

Synthesized according to General Procedure 6. Purified via trituration with diethyl ether. White solid (83%, 34 mg). ¹H NMR (400 MHz, cd₃od) δ 7.93 (d, J=8.3 Hz, 2H), 7.32 (d, J=8.6 Hz, 2H), 4.86-4.66 (m, 2H), 3.85 (d, J=5.6 Hz, 1H), 2.67 (t, J=7.7 Hz, 2H), 2.37-2.24 (m, 1H), 1.65 (p, J=7.7 Hz, 2H), 1.40-1.24 (m, 14H), 1.20-1.10 (m, 6H), 0.89 (t, J=6.9 Hz, 3H). ¹³C NMR (101 MHz, cd₃od) δ 177.78, 170.11, 169.57, 148.10, 130.08, 128.29, 125.17, 59.70, 36.83, 36.49, 33.04, 32.40, 31.56, 30.68, 30.67, 30.54, 30.42, 30.29, 23.71, 18.72, 18.00, 14.45. HRMS: (ESI) [M+H]+ calc. for C₂₄H₃₉N₄O₂, 415.3068, observed, 415.3064.

### 5-amino-N-((3-(4-decylphenyl)-1,2,4-oxadiazol-5-yl)methyl)pentanamide hydrochloride (9g)

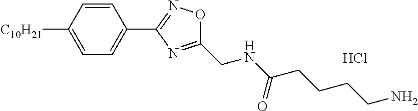

Synthesized according to General Procedure 6. Purification via trituration with ethyl acetate. White solid (86%, 102 mg). ¹H NMR (400 MHz, cd₃od) δ 7.95 (d, J=8.3 Hz, 2H), 7.34 (d, J=8.2 Hz, 2H), 4.70 (s, 2H), 2.97 (t, J=6.8 Hz, 2H), 2.69 (t, J=7.7 Hz, 2H), 2.42 (t, J=6.6 Hz, 2H), 1.80-1.62 (m, 6H), 1.42-1.23 (m, 14H), 0.90 (t, J=6.6 Hz, 3H). ¹³C NMR (101 MHz, cd₃od) δ 178.30, 175.86, 169.60, 148.12, 130.11, 128.31, 125.26, 40.36, 36.84, 36.51, 35.52, 33.06, 32.44, 30.69, 30.68, 30.55, 30.44, 30.30, 27.96, 23.72, 23.17, 14.43. HRMS: (ESI) [M+H]+ calc. for C₂₄H₃₉N₄O₂, 415.3068, observed, 415.3069.

### 2-amino-N-(2-(3-(4-decylphenyl)-1,2,4-oxadiazol-5-yl)ethyl)acetamide hydrochloride (9h)

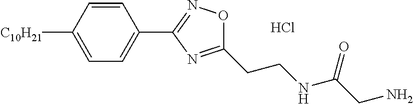

Synthesized according to General Procedure 6. Purified via trituration with ethyl acetate and diethyl ether. White solid (82%, 38 mg). ¹H NMR (400 MHz, cd₃od) δ 7.95 (d, J=8.3 Hz, 2H), 7.33 (d, J=8.1 Hz, 2H), 3.77 (t, J=6.6 Hz, 2H), 3.67 (s, 2H), 3.23 (t, J=6.6 Hz, 2H), 2.67 (t, J=7.7 Hz, 2H), 1.64 (p, J=7.3 Hz, 2H), 1.39-1.23 (m, 14H), 0.88 (t, J=7.0 Hz, 3H). ¹³C NMR (101 MHz, cd₃od) δ 179.31, 169.49, 167.56, 147.99, 130.07, 128.32, 125.39, 41.45, 37.63, 36.84, 33.06, 32.46, 30.71, 30.70, 30.57, 30.45, 30.31, 27.55, 23.73, 14.45. HRMS: (ESI) [M+NH₄]+ calc. for C₂₂H₃₈N₅O₂, 404.3020, observed, 404.3029.

### (S)—N-((3-(4-decylphenyl)-1,2,4-oxadiazol-5-yl)methyl)piperidine-2-carboxamide hydrochloride (9i)

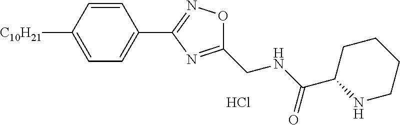

Synthesized according to General Procedure 6. White solid (88%, 44 mg). ¹H NMR (400 MHz, Methanol-d₄) δ 7.92 (d, J=8.2 Hz, 2H), 7.31 (d, J=8.1 Hz, 2H), 4.83-4.68 (m, 2H), 3.98 (dd, J=11.6, 3.3 Hz, 1H), 3.40 (dt, J=13.3, 2.8 Hz, 1H), 3.07 (td, J=12.5, 3.6 Hz, 1H), 2.66 (t, J=7.7 Hz, 2H), 2.32 (dt, J=12.8, 3.4 Hz, 1H), 2.01-1.57 (m, 7H), 1.39-1.20 (m, 14H), 0.88 (t, J=6.7 Hz, 3H). ¹³C NMR (101 MHz, Methanol-d₄) δ 176.35, 169.21, 168.20, 146.73, 128.69, 126.90, 123.76, 57.50, 43.58, 35.43, 35.08, 31.64, 31.01, 29.28, 29.27, 29.14, 29.02, 28.89, 27.04, 22.31, 21.55, 21.43, 13.04.

### (R)—N-((3-(4-decylphenyl)-1,2,4-oxadiazol-5-yl)methyl)pyrrolidine-3-carboxamide hydrochloride (9j)

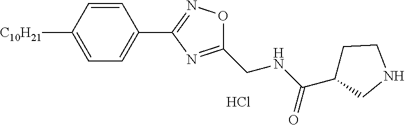

Synthesized according to General Procedure 6. Purified via trituration with ethyl acetate and diethyl ether. White solid (69%, 132 mg). ¹H NMR (400 MHz, cd₃od) δ 7.92 (d, J=8.2 Hz, 2H), 7.32 (d, J=8.0 Hz, 2H), 4.78-4.64 (m, 2H), 3.59-3.45 (m, 2H), 3.43-3.33 (m, 3H), 2.67 (t, J=7.7 Hz, 2H), 2.47-2.34 (m, 1H), 2.30-2.18 (m, 1H), 1.64 (p, J=7.5 Hz, 2H), 1.41-1.20 (m, 14H), 0.89 (t, J=6.7 Hz, 3H). ¹³C NMR (101 MHz, cd₃od) δ 178.10, 174.69, 169.55, 148.08, 130.09, 128.30, 125.20, 48.63, 46.55, 43.63, 36.83, 36.63, 33.04, 32.41, 30.68, 30.67, 30.54, 30.42, 30.29, 30.20, 23.71, 14.44. HRMS: (ESI) [M+H]+ calc. for C₂₄H₃₇N₄O₂, 413.2911, observed, 413.2904.

### (S)—N-((3-(4-decylphenyl)-1,2,4-oxadiazol-5-yl)methyl)-2-(pyrrolidin-3-yl)acetamide hydrochloride (9k)

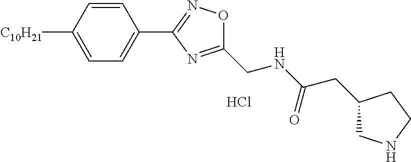

Synthesized according to General Procedure 6. Purification via trituration with ethyl acetate. White solid (94%, 182 mg). ¹H NMR (400 MHz, cd₃od) δ 7.95 (d, J=8.3 Hz, 2H), 7.34 (d, J=8.3 Hz, 2H), 4.71 (s, 2H), 3.55 (dd, J=11.7, 7.6 Hz, 1H), 3.49-3.37 (m, 1H), 3.31-3.22 (m, 1H), 3.03 (dd, J=11.7, 8.9 Hz, 1H), 2.81-2.65 (m, 3H), 2.65-2.49 (m, 2H), 2.35-2.23 (m, 1H), 1.85-1.75 (m, 1H), 1.66 (q, J=7.0 Hz, 2H), 1.41-1.23 (m, 14H), 0.91 (t, J=6.8 Hz, 3H). ¹³C NMR (101 MHz, cd₃od) δ 178.24, 174.07, 169.56, 148.06, 130.09, 128.29, 125.23, 50.90, 46.19, 38.71, 36.83, 36.49, 36.22, 33.04, 32.41, 30.97, 30.68, 30.67, 30.54, 30.42, 30.30, 23.71, 14.44. HRMS: (ESI) [M+H]+ calc. for C₂₅H₃₉N₄O₂, 427.3068, observed, 427.3071.

### N-((3-(4-decylphenyl)-1,2,4-oxadiazol-5-yl)methyl)-2-(methylamino)acetamide hydrochloride (9l)

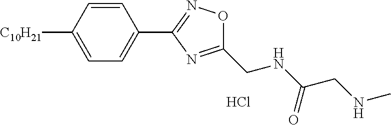

Synthesized according to General Procedure 6. Purified via trituration with ethyl acetate and diethyl ether. White solid (70%, 50 mg). ¹H NMR (400 MHz, cd₃od) δ 7.93 (d, J=8.3 Hz, 2H), 7.33 (d, J=8.2 Hz, 2H), 4.78 (s, 2H), 3.96 (s, 2H), 2.77 (s, 3H), 2.67 (t, J=7.7 Hz, 2H), 1.64 (p, J=7.3 Hz, 2H), 1.40-1.28 (m, 14H), 0.89 (t, J=6.8 Hz, 3H). ¹³C NMR (101 MHz, cd₃od) δ 177.76, 169.61, 167.17, 148.15, 130.10, 128.31, 125.15, 50.52, 36.83, 36.42, 33.62, 33.05, 32.41, 30.68, 30.67, 30.54, 30.42, 30.29, 23.71, 14.43. HRMS: (ESI) [M+H]+ calc. for C₂₂H₃₅N₄O₂, 387.2755, observed, 387.2738.

### (S)—N-((3-(4-decylphenyl)-1,2,4-oxadiazol-5-yl)methyl)pyrrolidine-2-carboxamide hydrochloride (9m)

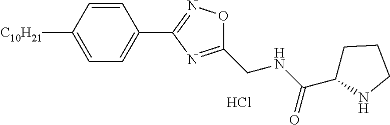

Synthesized according to General Procedure 6. White solid (94%, 160 mg). ¹H NMR (400 MHz, Methanol-d₄) δ 7.93 (d, J=8.2 Hz, 2H), 7.32 (d, J=8.0 Hz, 2H), 4.84-4.72 (m, 2H), 4.43 (dd, J=8.6, 6.6 Hz, 1H), 3.46-3.33 (m, 2H), 2.67 (t, J=7.7 Hz, 2H), 2.57-2.47 (m, 1H), 2.21-2.03 (m, 3H), 1.64 (p, J=7.1 Hz, 2H), 1.31 (d, J=24.5 Hz, 14H), 0.89 (t, J=6.8 Hz, 3H). ¹³C NMR (101 MHz, Methanol-d₄) δ 177.76, 170.37, 169.60, 148.16, 130.11, 128.30, 125.15, 61.14, 47.39, 36.83, 36.72, 33.05, 32.42, 30.87, 30.70, 30.68, 30.54, 30.43, 30.29, 24.98, 23.72, 14.43. HRMS: (ESI) [M+H]+ calc. for C₂₄H₃₇N₄O₂, 413.2911, observed, 413.2909.

### (2S,3S)-2-amino-N-((3-(4-decylphenyl)-1,2,4-oxadiazol-5-yl)methyl)-3-methylpentanamide hydrochloride (9n)

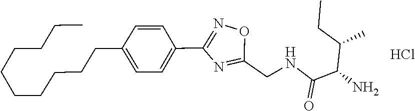 [SMILES: CCCCCCCCCCc1ccc(-c2noc(CNC(=O)[C@@H](N)[C@@H](C)CC)n2)cc1 ; Cl]

Synthesized according to General Procedure 6. Purified via trituration with diethyl ether. White solid (87%, 105 mg). ¹H NMR (400 MHz, cd₃od) δ 7.93 (d, J=8.0 Hz, 2H), 7.32 (d, J=8.0 Hz, 2H), 4.92-4.60 (m, 2H), 3.88 (d, J=5.2 Hz, 1H), 2.68 (t, J=7.7 Hz, 2H), 2.07-1.96 (m, 1H), 1.74-1.59 (m, 3H), 1.39-1.23 (m, 14H), 1.11 (d, J=6.9 Hz, 3H), 1.03 (t, J=7.4 Hz, 3H), 0.89 (t, J=6.7 Hz, 3H). ¹³C NMR (101 MHz, cd₃od) δ 177.81, 170.05, 169.59, 148.16, 130.10, 128.30, 125.18, 59.01, 38.13, 36.83, 36.50, 33.05, 32.42, 30.68, 30.68, 30.54, 30.43, 30.29, 25.52, 23.72, 15.01, 14.44, 11.83. HRMS: (ESI) [M+H]+ calc. for C₂₅H₄₁N₄O₂, 429.3224, observed, 429.3231.

### (S)—N-((3-(4-decylphenyl)-1,2,4-oxadiazol-5-yl)methyl)-2-(piperidin-3-yl)acetamide hydrochloride (9o)

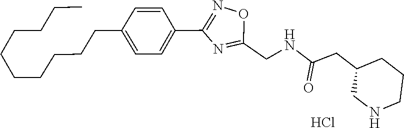 [SMILES: CCCCCCCCCCc1ccc(-c2noc(CNC(=O)C[C@@H]3CCCNC3)n2)cc1 ; Cl]

Synthesized according to General Procedure 6. Purified via trituration with diethyl ether. White solid (81%, 100 mg). ¹H NMR (400 MHz, cd₃od) δ 8.95 (t, J=5.8 Hz, 1H), 7.94 (d, J=8.0 Hz, 2H), 7.33 (d, J=8.0 Hz, 2H), 4.70 (s, 2H), 3.52-3.35 (m, 2H), 2.94 (td, J=12.7, 3.2 Hz, 1H), 2.81 (t, J=11.9 Hz, 1H), 2.68 (t, J=7.7 Hz, 2H), 2.41-2.24 (m, 3H), 2.04-1.90 (m, 2H), 1.88-1.73 (m, 1H), 1.65 (p, J=7.3 Hz, 2H), 1.45-1.22 (m, 15H), 0.90 (t, J=6.7 Hz, 3H). ¹³C NMR (101 MHz, cd₃od) δ 178.21, 173.71, 169.52, 148.02, 130.08, 128.28, 125.22, 49.35, 45.18, 40.17, 36.83, 36.53, 33.03, 32.59, 32.40, 30.68, 30.66, 30.54, 30.42, 30.30, 29.33, 23.70, 23.21, 14.45. HRMS: (ESI) [M+H]+ calc. for C₂₆H₄₁N₄O₂, 441.3224, observed, 441.3232.

### N-((3-(4-decylphenyl)-1,2,4-oxadiazol-5-yl)methyl)piperidine-4-carboxamide hydrochloride (9p)

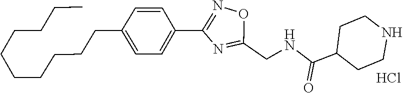 [SMILES: CCCCCCCCCCc1ccc(-c2noc(CNC(=O)C3CCNCC3)n2)cc1 ; Cl]

Synthesized according to General Procedure 6. via trituration with ethyl acetate and diethyl ether. White solid (70%, 97 mg). H NMR (400 MHz, cd₃od) δ 7.93 (d, J=8.3 Hz, 2H), 7.33 (d, J=8.2 Hz, 2H), 4.69 (s, 2H), 3.45 (dt, J=13.0, 4.0 Hz, 2H), 3.10 (td, J=12.7, 3.3 Hz, 2H), 2.78-2.64 (m, 3H), 2.16-2.05 (m, 2H), 2.02-1.87 (m, 2H), 1.65 (p, J=7.5 Hz, 2H), 1.41-1.22 (m, 14H), 0.89 (t, J=6.6 Hz, 3H). ¹³C NMR (101 MHz, cd₃od) δ 178.21, 176.33, 169.57, 148.08, 130.09, 128.30, 125.25, 44.21, 40.41, 36.83, 36.53, 33.05, 32.42, 30.69, 30.68, 30.55, 30.43, 30.29, 26.48, 23.72, 14.43. HRMS: (ESI) [M+H]+ calc. for C₂₅H₃₉N₄O₂, 427.3068, observed, 427.3070.

### (R)—N-((3-(4-decylphenyl)-1,2,4-oxadiazol-5-yl)methyl)morpholine-2-carboxamide hydrochloride (9q)

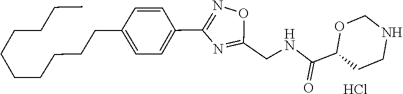 [SMILES: CCCCCCCCCCc1ccc(-c2noc(CNC(=O)[C@H]3CCNCO3)n2)cc1 ; Cl]

Synthesized according to General Procedure 6. Purified via trituration with ethyl acetate and diethyl ether. White solid (69%, 36 mg). ¹H NMR (400 MHz, cd₃od) δ 7.95 (d, J=8.3 Hz, 2H), 7.34 (d, J=8.2 Hz, 2H), 4.73 (s, 2H), 4.11 (dd, J=10.5, 2.9 Hz, 1H), 4.02 (dt, J=11.4, 2.5 Hz, 1H), 3.79-3.66 (m, 1H), 3.24 (dd, J=12.7, 2.9 Hz, 1H), 2.91-2.82 (m, 2H), 2.81-2.65 (m, 3H), 1.66 (p, J=7.2 Hz, 2H), 1.42-1.30 (m, 14H), 0.91 (t, J=6.7 Hz, 3H). ¹³C NMR (101 MHz, cd₃od) δ 177.97, 172.89, 169.57, 148.03, 130.07, 128.34, 125.25, 76.79, 68.07, 48.65, 45.72, 36.84, 36.05, 33.05, 32.42, 30.69, 30.69, 30.55, 30.43, 30.30, 23.72, 14.45. HRMS: (ESI) [M+H]+ calc. for C₂₄H₃₇N₄O₃, 429.2860, observed, 429.2865.

### (1R,3S)-3-amino-N-((3-(4-decylphenyl)-1,2,4-oxadiazol-5-yl)methyl)cyclohexane-1-carboxamide hydrochloride (9r)

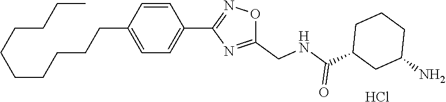 [SMILES: CCCCCCCCCCc1ccc(-c2noc(CNC(=O)[C@@H]3CCC[C@H](N)C3)n2)cc1 ; Cl]

Synthesized according to General Procedure 6. Purification via trituration with diethyl ether. White solid (86%, 108 mg). ¹H NMR (400 MHz, cd₃od) δ 7.91 (d, J=8.2 Hz, 2H), 7.29 (d, J=8.1 Hz, 2H), 4.67 (s, 2H), 3.21 (tt, J=11.7, 3.9 Hz, 1H), 2.64 (t, J=7.7 Hz, 2H), 2.52 (tt, J=11.8, 3.3 Hz, 1H), 2.22-2.13 (m, 1H), 2.11-2.01 (m, 1H), 1.99-1.87 (m, 2H), 1.68-1.20 (m, 20H), 0.87 (t, J=6.9 Hz, 3H). ¹³C NMR (101 MHz, cd₃od) δ 176.80, 176.00, 168.14, 146.60, 128.67, 126.91, 123.87, 49.26, 42.43, 35.45, 35.13, 32.54, 31.66, 31.03, 29.71, 29.31, 29.30, 29.17, 29.05, 28.92, 28.01, 22.98, 22.33, 13.09. HRMS: (ESI) [M+H]+ calc. for C₂₆H₄₁N₄O₂, 441.3224, observed, 441.3217.

### (S)-2-amino-N-((3-(4-decylphenyl)-1,2,4-oxadiazol-5-yl)methyl)-3-hydroxypropanamide hydrochloride (9s)

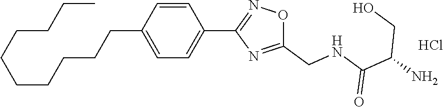 [SMILES: CCCCCCCCCCc1ccc(-c2noc(CNC(=O)[C@@H](N)CO)n2)cc1 ; Cl]

Synthesized according to General Procedure 6. Purified via trituration with ethyl acetate. White solid (75%, 94 mg). ¹H NMR (400 MHz, cd₃od) δ 7.95 (d, J=8.3 Hz, 2H), 7.35 (d, J=8.1 Hz, 2H), 4.85-4.73 (m, 2H), 4.10 (dd, J=6.5, 4.2 Hz, 1H), 4.04 (dd, J=11.6, 4.2 Hz, 1H), 3.94 (dd, J=11.6, 6.4 Hz, 1H), 2.69 (t, J=7.7 Hz, 2H), 1.67 (p, J=7.5 Hz, 2H), 1.42-1.23 (m, 14H), 0.91 (t, J=6.6 Hz, 3H). ¹³C NMR (101 MHz, cd₃od) δ 177.85, 169.59, 168.92, 148.19, 130.12, 128.32, 125.15, 61.60, 56.34, 36.83, 36.60, 33.05, 32.43, 30.69, 30.67, 30.55, 30.43, 30.29, 23.72, 14.43. HRMS: (ESI) [M+H]+ calc. for C₂₂H₃₅N₄O₃, 403.2704, observed, 403.2714.

### (R)—N-((3-(4-decylphenyl)-1,2,4-oxadiazol-5-yl)methyl)piperidine-2-carboxamide hydrochloride (9t)

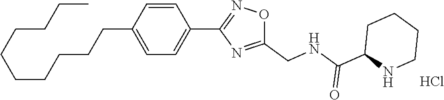 [SMILES: CCCCCCCCCCc1ccc(-c2noc(CNC(=O)[C@H]3CCCCN3)n2)cc1 ; Cl]

¹H NMR (400 MHz, CD₃OD) δ 7.93 (d, J=8.5 Hz, 2H), 7.33 (d, J=8.6 Hz, 2H), 4.70 (d, J=1.2 Hz, 2H), 3.40-3.34 (m, 1H), 3.14-3.07 (m, 1H), 2.73-2.64 (m, 3H), 2.02-1.94 (m, 1H), 1.91-1.84 (m, 1H), 1.70-1.58 (m, 3H), 1.57-1.44 (m, 3H), 1.38-1.27 (m, 14H), 0.88 (t, J=6.8 Hz, 3H). ¹³C NMR (101 MHz, CD₃OD) δ 178.19, 175.96, 169.58, 148.09, 130.09, 128.32, 125.28, 60.59, 46.27, 36.84, 36.34, 33.06, 32.43, 30.82, 30.71, 30.69, 30.55, 30.44, 30.30, 26.38, 24.93, 23.73, 14.43. HRMS: (ESI) [M+H]+ calc. for C₂₅H₃₉N₄O₂, 427.3068, observed, 427.3060.

### N-((3-(4-decylphenyl)-1,2,4-oxadiazol-5-yl)methyl)azepane-2-carboxamide hydrochloride (9u)

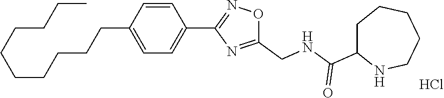 [SMILES: CCCCCCCCCCc1ccc(-c2noc(CNC(=O)C3CCCCCN3)n2)cc1 ; Cl]

Synthesized according to General Procedure 6. Purified via trituration with diethyl ether. White solid (78%, 99 mg). ¹H NMR (400 MHz, cd₃od) δ 7.93 (d, J=8.3 Hz, 2H), 7.33 (d, J=8.2 Hz, 2H), 4.84-4.70 (m, 2H), 4.15 (dd, J=9.7, 3.5 Hz, 1H), 3.40-3.32 (m, 1H), 3.30-3.19 (m, 1H), 2.68 (t, J=7.7 Hz, 2H), 2.43-2.31 (m, 1H), 2.13-2.00 (m, 1H), 1.96-1.59 (m, 8H), 1.40-1.24 (m, 14H), 0.89 (t, J=6.7 Hz, 3H). ¹³C NMR (101 MHz, cd₃od) δ 177.79, 171.31, 169.62, 148.18, 130.12, 128.30, 125.17, 60.82, 46.84, 36.83, 36.64, 33.05, 32.42, 31.07, 30.69, 30.68, 30.54, 30.43, 30.29, 27.33, 26.40, 26.17, 23.72, 14.43. HRMS: (ESI) [M+H]+ calc. for C₂₆H₄₁N₄O₂, 441.3224, observed, 441.3229.

### (2S,4S)—N-((3-(4-decylphenyl)-1,2,4-oxadiazol-5-yl)methyl)-4-hydroxypyrrolidine-2-carboxamide hydrochloride (9v)

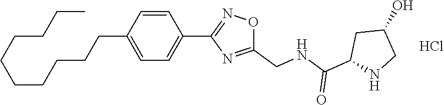 [SMILES: CCCCCCCCCCc1ccc(-c2noc(CNC(=O)[C@@H]3C[C@H](O)CN3)n2)cc1 ; Cl]

Synthesized according to General Procedure 6. Purified via trituration with diethyl ether and ethyl acetate. White solid (83%, 32 mg). ¹H NMR (400 MHz, cd₃od) δ 7.93 (d, J=8.3 Hz, 2H), 7.31 (d, J=8.2 Hz, 2H), 4.82-4.77 (m, 2H), 4.61-4.48 (m, 2H), 3.40 (d, J=3.2 Hz, 2H), 2.76-2.64 (m, 3H), 2.30-2.20 (m, 1H), 1.64 (p, J=7.4 Hz, 2H), 1.41-1.22 (m, 14H), 0.89 (t, J=6.8 Hz, 3H). ¹³C NMR (101 MHz, cd₃od) δ 177.68, 170.45, 169.58, 148.08, 130.07, 128.33, 125.15, 70.30, 59.97, 54.51, 39.28, 36.83, 33.03, 32.40, 30.68, 30.66, 30.54, 30.42, 30.29, 23.70, 14.44. HRMS: (ESI) [M+H]+ calc. for C₂₄H₃₇N₄O₃, 429.2860, observed, 429.2868.

### (2S,4R)—N-((3-(4-decylphenyl)-1,2,4-oxadiazol-5-yl)methyl)-4-hydroxypyrrolidine-2-carboxamide hydrochloride 9w

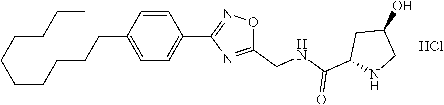 [SMILES: CCCCCCCCCCc1ccc(-c2noc(CNC(=O)[C@@H]3C[C@@H](O)CN3)n2)cc1 ; Cl]

Synthesized according to General Procedure 6. Purified via trituration with ethyl acetate and diethyl ether. White solid (81%, 79 mg). ¹H NMR (400 MHz, cd₃od) δ 7.93 (d, J=8.3 Hz, 2H), 7.34 (d, J=8.3 Hz, 2H), 4.82-4.73 (m, 2H), 4.66-4.54 (m, 2H), 3.43 (dd, J=12.1, 3.5 Hz, 1H), 3.36-3.32 (m, 1H), 2.68 (t, J=7.7 Hz, 2H), 2.55 (ddt, J=13.5, 7.6, 1.7 Hz, 1H), 2.17 (ddd, J=13.5, 10.4, 4.1 Hz, 1H), 1.65 (p, J=7.5 Hz, 2H), 1.43-1.21 (m, 14H), 0.89 (t, J=6.6 Hz, 3H). ¹³C NMR (101 MHz, cd₃od) δ 177.73, 170.34, 169.63, 148.22, 130.14, 128.32, 125.14, 71.17, 59.98, 55.17, 39.84, 36.84, 36.71, 33.06, 32.43, 30.70, 30.68, 30.55, 30.43, 30.29, 23.72, 14.43. HRMS: (ESI) [M+H]+ calc. for C₂₄H₃₇N₄O₃, 429.2860, observed, 429.2862.

### (S)—N-((3-(4-decylphenyl)-1,2,4-oxadiazol-5-yl)methyl)pyrrolidine-3-carboxamide hydrochloride (9x)

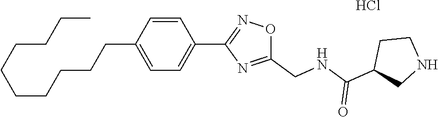 [SMILES: Cl ; CCCCCCCCCCc1ccc(-c2noc(CNC(=O)[C@H]3CCNC3)n2)cc1]

Synthesized according to General Procedure 6. Purified via trituration with ethyl acetate and diethyl ether. White solid (78%, 151 mg). ¹H NMR (400 MHz, cd₃od) δ 7.95 (d, J=8.3 Hz, 2H), 7.35 (d, J=8.3 Hz, 2H), 4.81-4.67 (m, 2H), 3.60 (dd, J=11.8, 5.4 Hz, 1H), 3.49 (dd, J=11.9, 7.9 Hz, 1H), 3.45-3.35 (m, 3H), 2.70 (t, J=7.7 Hz, 2H), 2.49-2.36 (m, 1H), 2.33-2.21 (m, 1H), 1.67 (p, J=7.2 Hz, 2H), 1.42-1.24 (m, 14H), 0.92 (t, J=6.7 Hz, 3H). ¹³C NMR (101 MHz, cd₃od) δ 178.13, 174.74, 169.59, 148.13, 130.11, 128.31, 125.22, 48.67, 46.56, 43.62, 36.83, 36.64, 33.05, 32.42, 30.68, 30.67, 30.54, 30.42, 30.29, 30.21, 23.71, 14.43. HRMS: (ESI) [M+H]+ calc. for C₂₄H₃₇N₄O₂, 413.2911, observed, 413.2903.

### (R)-4-amino-N-((3-(4-decylphenyl)-1,2,4-oxadiazol-5-yl)methyl)-3-hydroxybutanamide hydrochloride (9y)

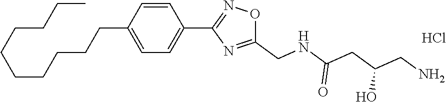 [SMILES: CCCCCCCCCCc1ccc(-c2noc(CNC(=O)C[C@@H](O)CN)n2)cc1 ; Cl]

Synthesized according to General Procedure 6. Purified via trituration with ethyl acetate and diethyl ether. White solid (70%, 90 mg). ¹H NMR (400 MHz, cd₃od) δ 7.93 (d, J=8.3 Hz, 2H), 7.32 (d, J=8.2 Hz, 2H), 4.78-4.62 (m, 2H), 4.30-4.19 (m, 1H), 3.16 (dd, J=12.8, 3.2 Hz, 1H), 2.94 (dd, J=12.8, 9.2 Hz, 1H), 2.67 (t, J=7.7 Hz, 2H), 2.60-2.57 (m, 2H), 1.65 (p, J=7.5 Hz, 2H), 1.38-1.23 (m, 14H), 0.90 (t, J=7.0 Hz, 3H). ¹³C NMR (101 MHz, cd₃od) δ 178.22, 173.00, 169.55, 148.09, 130.10, 128.32, 125.21, 65.99, 45.46, 41.98, 36.84, 36.49, 33.05, 32.42, 30.69, 30.68, 30.55, 30.43, 30.30, 23.72, 14.43. HRMS: (ESI) [M+H]+ calc. for C₂₃H₃₇N₄O₃, 417.2860, observed, 417.2866.

### (S)—N-((3-(4-decylphenyl)-1,2,4-oxadiazol-5-yl)methyl)azetidine-2-carboxamide 2,2,2-trifluoroacetate (9z)

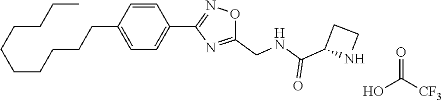 [SMILES: CCCCCCCCCCc1ccc(-c2noc(CNC(=O)[C@@H]3CCN3)n2)cc1 ; O=C(O)C(F)(F)F]

Synthesized according to General Procedure 5. Purification via trituration with diethyl ether. White solid (69%, 66 mg). ¹H NMR (400 MHz, CDCl₃) δ 8.48-7.80 (m, 3H), 7.27 (d, J=8.1 Hz, 2H), 4.89-4.68 (m, 3H), 3.95 (q, J=8.3 Hz, 1H), 3.89-3.79 (m, 1H), 2.65 (t, J=7.7 Hz, 2H), 2.56-2.43 (m, 2H), 1.63 (q, J=7.5 Hz, 2H), 1.48 (s, 9H), 1.36-1.22 (m, 14H), 0.88 (t, J=6.8 Hz, 3H). ¹³C NMR (101 MHz, CDCl₃) δ 175.65, 172.12, 168.53, 157.57, 146.76, 128.97, 127.52, 123.87, 81.35, 62.30, 47.41, 36.06, 35.71, 31.99, 31.32, 29.70, 29.67, 29.57, 29.42, 29.37, 28.39, 22.78, 19.64, 14.23. HRMS: (ESI) [M+H]+ calc. for C₂₈H₄₃N₄O₄, 499.3279, observed, 499.3277.

### methyl (S)-5-amino-6-(((3-(4-decylphenyl)-1,2,4-oxadiazol-5-yl)methyl)amino)-6-oxohexanoate hydrochloride (9aa)

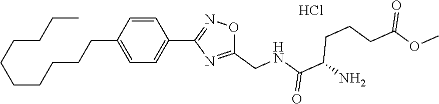 [SMILES: CCCCCCCCCCc1ccc(-c2noc(CNC(=O)[C@@H](N)CCCC(=O)OC)n2)cc1 ; Cl]

Synthesized according to General Procedure 6. Purification via trituration with diethyl ether. White solid (58%, 32 mg). ¹H NMR (400 MHz, cd₃od) δ 7.95 (d, J=8.3 Hz, 2H), 7.35 (d, J=8.1 Hz, 2H), 4.92-4.68 (m, 2H), 4.04 (t, J=6.3 Hz, 1H), 3.67 (s, 3H), 2.70 (t, J=7.7 Hz, 2H), 2.47 (t, J=7.1 Hz, 2H), 2.08-1.92 (m, 2H), 1.92-1.77 (m, 2H), 1.67 (p, J=7.2 Hz, 2H), 1.43-1.30 (m, 14H), 0.91 (t, J=6.8 Hz, 3H). ¹³C NMR (101 MHz, cd₃od) δ 177.79, 174.91, 170.56, 169.60, 148.14, 130.11, 128.33, 125.18, 54.20, 52.16, 36.83, 36.51, 34.00, 33.05, 32.42, 31.90, 30.68, 30.67, 30.55, 30.43, 30.29, 23.72, 21.01, 14.44. HRMS: (ESI) [M+H]+ calc. for C₂₆H₄₁N₄O₄, 473.3122, observed, 473.3104.

### N-((3-(4-decylphenyl)-1,2,4-oxadiazol-5-yl)methyl)-2-(dimethylamino)acetamide hydrochloride (9ab)

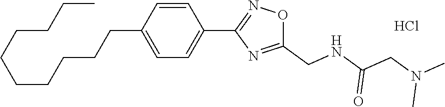 [SMILES: CCCCCCCCCCc1ccc(-c2noc(CNC(=O)CN(C)C)n2)cc1 ; Cl]

Synthesized according to General Procedure 6. Purified via trituration with ethyl acetate and diethyl ether. White solid (98%, 82 mg). ¹H NMR (400 MHz, cd₃od) δ 7.92 (d, J=8.3 Hz, 2H), 7.32 (d, J=8.3 Hz, 2H), 4.85 (s, 2H), 4.13 (s, 2H), 2.99 (s, 6H), 2.67 (t, J=7.7 Hz, 2H), 1.64 (p, J=7.1 Hz, 2H), 1.39-1.21 (m, 14H), 0.89 (t, J=6.7 Hz, 3H). ¹³C NMR (101 MHz, cd₃od) δ 177.65, 169.59, 166.39, 148.13, 130.10, 128.30, 125.12, 59.14, 44.50, 36.83, 36.48, 33.04, 32.40, 30.69, 30.68, 30.54, 30.42, 30.29, 23.71, 14.44. HRMS: (ESI) [M+H]+ calc. for C₂₃H₃₇N₄O₂, 401.2911, observed, 401.2911.

### (S)-2-amino-N-((3-(4-decylphenyl)-1,2,4-oxadiazol-5-yl)methyl)-3-phenylpropanamide hydrochloride (9ac)

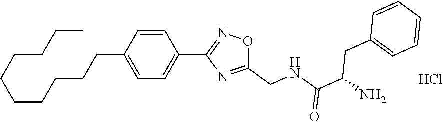 [SMILES: CCCCCCCCCCc1ccc(-c2noc(CNC(=O)[C@@H](N)Cc3ccccc3)n2)cc1 ; Cl]

Synthesized according to General Procedure 6. Purified via trituration with diethyl ether. White solid (46%, 64 mg). ¹H NMR (400 MHz, cd₃od) δ 7.94 (d, J=8.3 Hz, 2H), 7.39-7.21 (m, 8H), 4.82-4.62 (m, 3H), 4.21 (dd, J=7.6, 6.7 Hz, 1H), 3.29 (dd, J=14.1, 6.7 Hz, 1H), 3.14 (dd, J=14.0, 7.6 Hz, 1H), 2.68 (t, J=7.4 Hz, 2H), 1.65 (p, J=7.6 Hz, 3H), 1.39-1.22 (m, 18H), 0.88 (t, J=7.0 Hz, 2H). ¹³C NMR (101 MHz, cd₃od) δ 177.51, 170.11, 169.62, 148.17, 135.33, 130.56, 130.11, 130.08, 128.86, 128.34, 125.18, 55.74, 38.51, 36.82, 36.31, 33.02, 32.37, 30.66, 30.66, 30.52, 30.40, 30.27, 23.69, 14.41. HRMS: (ESI) [M+H]+ calc. for C₂₈H₃₉N₄O₂, 463.3068, observed, 463.3069.

### (S)-2-amino-N-((3-(4-decylphenyl)-1,2,4-oxadiazol-5-yl)methyl)-4-methylpentanamide hydrochloride (9ad)

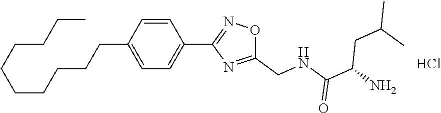 [SMILES: CCCCCCCCCCc1ccc(-c2noc(CNC(=O)[C@@H](N)CC(C)C)n2)cc1 ; Cl]

Synthesized according to General Procedure 6. Purified via trituration with diethyl ether. White solid (78%, 101 mg). ¹H NMR (400 MHz, cd₃od) δ 7.93 (d, J=8.2 Hz, 2H), 7.31 (d, J=8.1 Hz, 2H), 4.84-4.67 (m, 2H), 4.02 (dd, J=8.0, 6.0 Hz, 1H), 2.66 (t, J=7.7 Hz, 2H), 1.91-1.71 (m, 3H), 1.64 (p, J=7.2 Hz, 2H), 1.39-1.22 (m, 14H), 1.05 (dd, J=8.6, 5.9 Hz, 6H), 0.88 (t, J=6.7 Hz, 3H). ¹³C NMR (101 MHz, cd₃od) δ 177.74, 171.27, 169.56, 148.10, 130.07, 128.29, 125.16, 52.99, 41.69, 36.83, 36.54, 33.04, 32.40, 30.68, 30.67, 30.54, 30.42, 30.29, 25.37, 23.71, 23.00, 22.36, 14.45. HRMS: (ESI) [M+H]+ calc. for C₂₅H₄₁N₄O₂, 429.3224, observed, 429.3224.

### (2S,4R)—N-((3-(4-decylphenyl)-1,2,4-oxadiazol-5-yl)methyl)-4-fluoropyrrolidine-2-carboxamide hydrochloride (9ae)

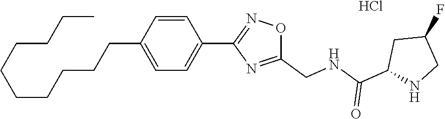 [SMILES: Cl ; CCCCCCCCCCc1ccc(-c2noc(CNC(=O)[C@@H]3C[C@@H](F)CN3)n2)cc1]

Synthesized according to General Procedure 6. Purified via trituration with ethyl acetate and diethyl ether. White solid (72%, 90 mg). ¹H NMR (400 MHz, cd₃od) δ 7.94 (d, J=8.3 Hz, 2H), 7.34 (d, J=8.1 Hz, 2H), 5.53 (dt, J=51.6, 3.5 Hz, 1H), 4.84-4.74 (m, 2H), 4.63 (dd, J=10.5, 7.6 Hz, 1H), 3.76-3.56 (m, 2H), 2.94-2.79 (m, 1H), 2.68 (t, J=7.7 Hz, 2H), 2.35 (dddd, J=39.0, 14.6, 10.6, 3.8 Hz, 1H), 1.65 (p, J=7.4 Hz, 2H), 1.40-1.23 (m, 14H), 0.89 (t, J=6.7 Hz, 3H). ¹³C NMR (101 MHz, cd₃od) δ 177.67, 169.58 (d, J=11.8 Hz), 148.23, 130.14, 128.32, 125.12, 93.81 (d, J=177.3 Hz), 59.85, 53.39 (d, J=23.6 Hz), 37.90 (d, J=22.1 Hz), 36.84, 36.73, 33.06, 32.43, 30.70, 30.69, 30.55, 30.43, 30.29, 23.72, 14.43. HRMS: (ESI) [M+H]+ calc. for C₂₄H₃₆FN₄O₂, 431.2817, observed, 431.2831.

### 1-amino-N-((3-(4-decylphenyl)-1,2,4-oxadiazol-5-yl)methyl)cyclopropane-1-carboxamide hydrochloride (9af)

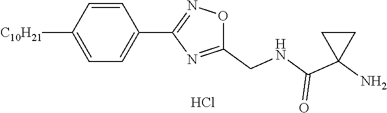

Synthesized according to General Procedure 8 with the following adaptation. The Boc protected product was immediately deprotected according to General Procedure 6 and purified by silica chromatography (10% methanol in dichloromethane). White solid (50%, 94 mg). ¹H NMR (400 MHz, Methanol-d₄) δ 7.92 (d, J=8.2 Hz, 2H), 7.31 (d, J=8.1 Hz, 2H), 4.70 (s, 2H), 2.66 (t, J=7.7 Hz, 2H), 1.69-1.59 (m, 4H), 1.55-1.48 (m, 2H), 1.36-1.24 (m, 14H), 0.88 (t, J=6.8 Hz, 3H). ¹³C NMR (101 MHz, Methanol-d₄) δ 177.97, 171.56, 169.52, 148.03, 130.05, 128.29, 125.17, 36.82, 36.76, 36.52, 33.03, 32.39, 30.68, 30.67, 30.54, 30.42, 30.30, 23.71, 14.46, 13.50. HRMS: (ESI) [M+H]+ calc. for C₂₃H₃₅N₄O₂, 399.2755, observed, 399.2742.

### (R)—N-((3-(4-decylphenyl)-1,2,4-oxadiazol-5-yl)methyl)piperidine-3-carboxamide hydrochloride (9ag)

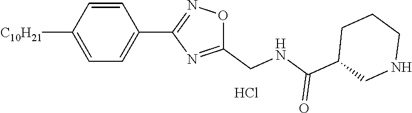

Synthesized according to General Procedure 8 with the following adaptation. The Boc protected product was immediately deprotected according to General Procedure 6 and purified by silica chromatography (10% methanol in dichloromethane). White solid (65%, 91 mg). ¹H NMR (400 MHz, Methanol-d₄) δ 7.95 (d, J=8.0 Hz, 2H), 7.34 (d, J=8.0 Hz, 2H), 4.73 (q, J=17.0 Hz, 2H), 3.39-3.21 (m, 3H), 3.19-3.08 (m, 1H), 3.00-2.90 (m, 1H), 2.69 (t, J=7.7 Hz, 2H), 2.18-2.09 (m, 1H), 2.04-1.81 (m, 3H), 1.67 (p, J=7.1 Hz, 2H), 1.38-1.22 (m, 14H), 0.91 (t, J=6.7 Hz, 3H). ¹³C NMR (101 MHz, Methanol-d₄) δ 176.75, 173.78, 168.16, 146.71, 128.69, 126.88, 123.81, 44.65, 43.67, 38.02, 35.42, 35.04, 31.64, 31.01, 29.27, 29.26, 29.13, 29.01, 28.88, 25.58, 22.30, 20.38, 13.02. HRMS: (ESI) [M+H]+ calc. for C₂₅H₃₉N₄O₂, 427.3068, observed, 427.3056.

### (1R,3R)-3-amino-N-((3-(4-decylphenyl)-1,2,4-oxadiazol-5-yl)methyl)cyclopentane-1-carboxamide hydrochloride (9ah)

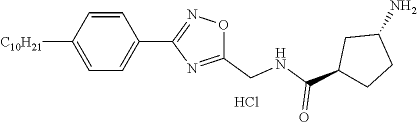

Synthesized according to General Procedure 8 with the following adaptation. The Boc protected product was immediately deprotected according to General Procedure 6 and purified by silica chromatography (10% methanol in dichloromethane). White solid (73%, 96 mg). ¹H NMR (400 MHz, Methanol-d₄) δ 7.95 (d, J=8.2 Hz, 2H), 7.34 (d, J=8.2 Hz, 2H), 4.71 (d, J=1.2 Hz, 2H), 3.79-3.71 (m, 1H), 3.14-3.06 (m, 1H), 2.69 (t, J=7.7 Hz, 2H), 2.33-2.25 (m, 1H), 2.21-1.93 (m, 2H), 2.09-1.94 (m, 2H), 1.90-1.81 (m, 1H), 1.66 (p, J=7.5 Hz, 2H), 1.38-1.23 (m, 14H), 0.90 (t, J=6.8 Hz, 3H). ¹³C NMR (101 MHz, Methanol-d₄) δ 177.95, 176.72, 168.17, 146.70, 128.69, 126.88, 123.82, 51.96, 43.16, 35.41, 35.29, 34.25, 31.63, 31.01, 30.39, 29.27, 29.26, 29.13, 29.01, 28.87, 28.69, 22.30, 13.01. HRMS: (ESI) [M+H]+ calc. for C₂₅H₃₉N₄O₂, 427.3068, observed, 427.3065.

### (S)-2-amino-1-((R)-3-(3-(4-decylphenyl)-1,2,4-oxadiazol-5-yl)pyrrolidin-1-yl)-3-methylbutan-1-one 2,2,2-trifluoroacetate (9ai)

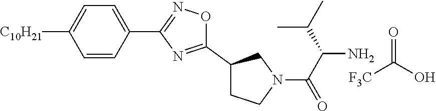

Synthesized according to General Procedure 8 with the following adaptation. The Boc protected product was immediately deprotected according to General Procedure 5 and purified by silica chromatography (10% methanol in dichloromethane). Colorless semisolid (89%, 59 mg). ¹H NMR (400 MHz, CD₃OD) δ 8.00-7.86 (m, 2H), 7.37-7.23 (m, 2H), 4.13-3.55 (m, 6H), 2.66 (t, J=7.7 Hz, 2H), 2.61-2.31 (m, 2H), 2.17-2.01 (m, 1H), 1.63 (p, J=6.9 Hz, 2H), 1.40-1.19 (m, 14H), 1.10-0.97 (m, 6H), 0.88 (t, J=6.7 Hz, 3H). ¹³C NMR (101 MHz, CD₃OD) δ 181.3, 180.8, 169.5, 148.0, 130.1, 128.3, 125.3, 58.6, 50.9, 50.6*, 46.7, 46.6*, 38.0, 36.8, 36.0, 33.1, 32.4, 31.9*, 31.3, 30.7, 30.6, 30.4, 30.3, 29.3, 23.7. 19.6, 19.5*, 17.7, 17.3*, 14.5. HRMS: (ESI) [M+H]+ calc. for C₂₇H₄₃N₄O₂, 455.3381, observed, 455.3404.

### ((S)-2-amino-1-((R)-3-(3-(4-decylphenyl)-1,2,4-oxadiazol-5-yl)pyrrolidin-1-yl)-3-hydroxypropan-1-one 2,2,2-trifluoroacetate (9aj)

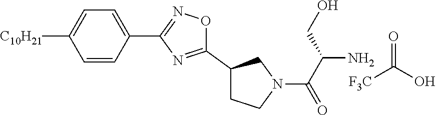

Synthesized according to General Procedure 8 with the following adaptation. The boc protected product was immediately deprotected according to General Procedure 5 and purified by silica chromatography (10% methanol in dichloromethane). White solid (47%, 35 mg). ¹H NMR (400 MHz, CD₃OD) δ 7.94 (d, J=7.9 Hz, 2H), 7.31 (d, J=7.9 Hz, 2H), 4.19-3.57 (m, 8H), 2.66 (t, J=7.7 Hz, 2H), 2.62-2.30 (m, 2H), 1.63 (p, J=7.1 Hz, 2H), 1.40-1.19 (m, 14H), 0.88 (t, J=6.7 Hz, 3H). ¹³C NMR (101 MHz, CD₃OD) δ 181.1, 180.8, 169.5, 148.1, 130.1, 128.3, 125.3, 125.3*, 63.8, 63.7*, 55.4, 50.7, 50.6*, 46.7, 46.7*, 37.9, 36.8, 36.0, 33.1, 32.4, 31.3, 30.7, 30.6, 30.4, 30.3, 29.4, 23.7, 14.5. HRMS: (ESI) [M+H]+ calc. for C₂₅H₃₉N₄O₃, 443.3017, observed, 443.3026.

### (S)-2-amino-1-((R)-3-(3-(4-decylphenyl)-1,2,4-oxadiazol-5-yl)pyrrolidin-1-yl)propan-1-one 2,2,2-trifluoroacetate (9ak)

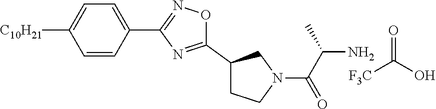

Synthesized according to General Procedure 8 with the following adaptation. The boc protected product was immediately deprotected according to General Procedure 5 and purified by silica chromatography (10% methanol in dichloromethane). White solid (41%, 30 mg). ¹H NMR (400 MHz, CD₃OD) δ 7.97-7.89 (m, 2H), 7.34-7.27 (m, 2H), 4.07-3.51 (m, 6H), 2.66 (t, J=7.5 Hz, 2H), 2.61-2.28 (m, 2H), 1.63 (p, J=6.8 Hz, 2H), 1.41-1.19 (m, 14H), 0.88 (t, J=6.8 Hz, 3H). ¹³C NMR (101 MHz, CD₃OD) δ 181.2, 180.9*, 175.5, 175.3*, 148.0, 148.0*, 130.1, 128.3, 128.3*, 125.3, 125.3*, 50.5, 50.4*, 49.1, 46.5, 46.4*, 38.0, 36.9, 36.0, 33.1, 32.4, 31.4, 30.7, 30.6, 30.5, 30.3, 29.5, 23.7, 20.0, 19.9*, 14.5. HRMS: (ESI) [M+H]+ calc. for C₂₅H₃₉N₄O₂, 427.3068, observed, 427.3069.

### (R)-2-amino-1-(3-(3-(4-decylphenyl)-1,2,4-oxadiazol-5-yl)pyrrolidin-1-yl)ethan-1-one 2,2,2-trifluoroacetate (9al)

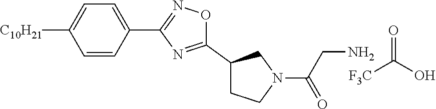

Synthesized according to General Procedure 8 with the following adaptation. The boc protected product was immediately deprotected according to General Procedure 5 and purified by silica chromatography (10% methanol in dichloromethane). Yellow solid (82%, 141 mg). ¹H NMR (400 MHz, CD₃OD) δ 7.98-7.84 (m, 2H), 7.31-7.20 (m, 2H), 4.06-3.80 (m, 5H), 3.78-3.58 (m, 2H), 2.62 (t, J=7.5 Hz, 2H), 2.58-2.25 (m, 2H), 1.60 (p, J=6.9 Hz, 2H), 1.36-1.18 (m, 14H), 0.87 (t, J=6.7 Hz, 3H). ¹³C NMR (101 MHz, CD₃OD) δ 180.9, 180.6*, 169.4, 163.2, 162.8*, 147.9, 130.0, 128.3, 125.2, 50.4, 49.6, 46.5, 45.9, 41.7, 41.7*, 37.8, 36.8, 36.1*, 33.0, 32.4*, 31.2, 30.7, 30.6, 30.4, 30.3, 23.7, 14.5. HRMS: (ESI) [M+H]+ calc. for C₂₄H₃₇N₄O₂, 413.2911, observed, 413.2889.

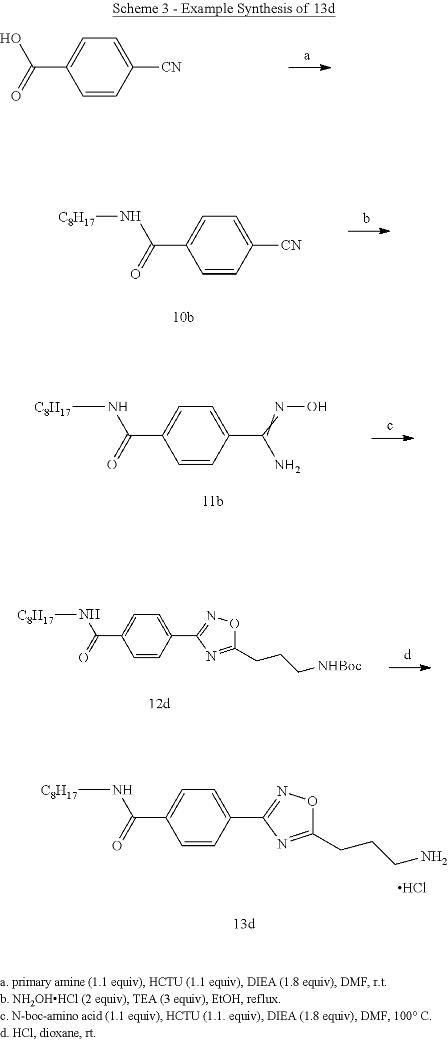

### N-butyl-4-cyanobenzamide (10a)

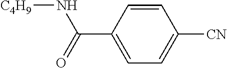

Synthesized according to General Procedure 8 using 4-cyanobenzonitrile. Purified by silica chromatography (50% ethyl acetate in hexanes). Clear oil (97%, 799 mg). ¹H NMR (400 MHz, Chloroform-d) δ 7.92 (d, J=8.4 Hz, 2H), 7.70 (d, J=8.4 Hz, 2H), 7.17 (t, J=5.7 Hz, 1H), 3.43 (q, J=6.9, 2H), 1.59 (q, J=7.9 Hz, 2H), 1.38 (h, J=7.3 Hz, 2H), 0.93 (t, J=7.4 Hz, 3H). ¹³C NMR (101 MHz, Chloroform-d) δ 165.96, 138.75, 132.22, 127.77, 118.05, 114.58, 40.08, 31.42, 20.09, 13.69.

### 4-cyano-N-octylbenzamide (10b)

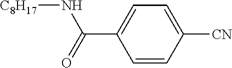

Synthesized according to General Procedure 8 using 4-cyanobenzonitrile. Purified by silica chromatography (50% ethyl acetate in hexanes). Clear oil (94%, 822 mg). ¹H NMR (400 MHz, Chloroform-d) δ 7.85 (d, J=8.3 Hz, 2H), 7.72 (d, J=8.3 Hz, 2H), 6.29 (t, J=5.9 Hz, 1H), 3.44 (q, J=7.3 Hz, 2H), 1.61 (p, J=7.4 Hz, 2H), 1.40-1.22 (m, 10H), 0.86 (t, J=6.8 Hz, 3H). ¹³C NMR (101 MHz, Chloroform-d) δ 165.82, 138.88, 132.53, 127.72, 118.17, 115.01, 40.52, 31.89, 29.65, 29.37, 29.31, 27.10, 22.76, 14.22. HRMS: (ESI) [M+H]+ calc. for C₁₆H₂₃N₂O, 259.1805, observed, 259.1804.

### N-butyl-4-(N′-hydroxycarbamimidoyl)benzamide (11a)

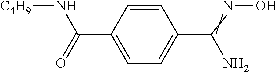

Synthesized according to General Procedure 3. Purified by silica chromatography (90% ethyl acetate in hexanes). White solid (61%, 554 mg). ¹H NMR (400 MHz, Methanol-d₄) δ 7.84 (d, J=8.5 Hz, 2H), 7.74 (d, J=8.5 Hz, 2H), 3.39 (t, J=7.2 Hz, 2H), 1.62 (p, J=7.3 Hz, 2H), 1.43 (h, J=7.3 Hz, 2H), 0.99 (t, J=7.4 Hz, 3H). ¹³C NMR (101 MHz, Methanol-d₄) δ 169.50, 154.45, 137.13, 136.79, 128.30, 127.25, 40.79, 32.61, 21.19, 14.14.

### 4-(N′-hydroxycarbamimidoyl)-N-octylbenzamide (11b)

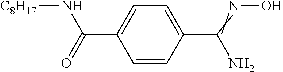

Synthesized according to General Procedure. Purified by silica chromatography (90% ethyl acetate in hexanes). White solid (52%, 555 mg). ¹H NMR (400 MHz, Methanol-d₄) δ 7.87 (d, J=8.3 Hz, 2H), 7.77 (d, J=8.5 Hz, 2H), 3.41 (t, J=7.2 Hz, 2H), 1.66 (p, J=7.1 Hz, 2H), 1.47-1.30 (m, 10H), 0.93 (t, J=6.8 Hz, 3H). ¹³C NMR (101 MHz, Methanol-d₄) δ 169.52, 162.59, 137.16, 136.83, 128.30, 127.26, 41.09, 32.99, 30.48, 30.43, 30.37, 28.11, 23.70, 14.41.

### tert-butyl (R)-3-(3-(4-(butylcarbamoyl)phenyl)-1,2,4-oxadiazol-5-yl)pyrrolidine-1-carboxylate (12a)

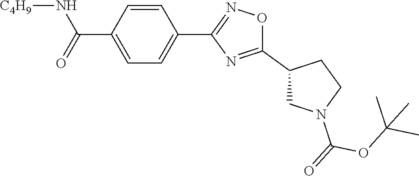

Synthesized according to General Procedure 4. Purified by silica chromatography (60% ethyl acetate in hexanes). Yellow solid (67%, 329 mg). ¹H NMR (400 MHz, Chloroform-d) δ 8.08 (d, J=8.1 Hz, 2H), 7.85 (d, J=8.2 Hz, 2H), 6.54 (t, J=5.7 Hz, 1H), 3.88-3.54 (m, 4H), 3.52-3.40 (m, 3H), 2.43-4.28 (m, 2H), 1.59 (p, J=8.0, 2H), 1.45 (s, 9H), 1.38 (h, J=7.4 Hz, 2H), 0.93 (t, J=7.3 Hz, 3H). ¹³C NMR (101 MHz, Chloroform-d) δ 179.87, 167.78, 166.82, 154.27, 137.37, 129.25, 127.63, 127.55, 79.90, 49.44, 49.23, 45.34, 45.11, 40.04, 36.65, 35.86, 31.76, 30.44, 29.67, 28.55, 20.25, 13.87.

### tert-butyl (3-(3-(4-(butylcarbamoyl)phenyl)-1,2,4-oxadiazol-5-yl)propyl)carbamate (12b)

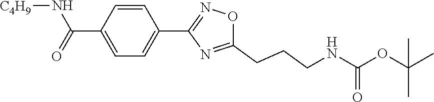

Synthesized according to General Procedure 4. Purified by silica chromatography (55% ethyl acetate in hexanes). White solid (52%, 251 mg). ¹H NMR (400 MHz, Chloroform-d) δ 8.10 (d, J=8.4 Hz, 2H), 7.85 (d, J=8.4 Hz, 2H), 6.32 (t, J=5.8 Hz, 1H), 4.82 (s, 1H), 3.46 (td, J=7.2, 5.7 Hz, 2H), 3.28 (q, J=6.5 Hz, 2H), 2.99 (t, J=7.5 Hz, 2H), 2.08 (p, J=7.1 Hz, 2H), 1.67-1.54 (m, 2H), 1.46-1.36 (m, 11H), 0.95 (t, J=7.3 Hz, 3H). ¹³C NMR (101 MHz, Chloroform-d) δ 179.78, 167.71, 166.84, 156.09, 137.27, 129.56, 127.67, 127.52, 79.56, 40.07, 39.85, 31.82, 28.50, 27.05, 24.17, 20.30, 13.91.

### tert-butyl (R)-3-(3-(4-(octylcarbamoyl)phenyl)-1,2,4-oxadiazol-5-yl)pyrrolidine-1-carboxylate (12c)

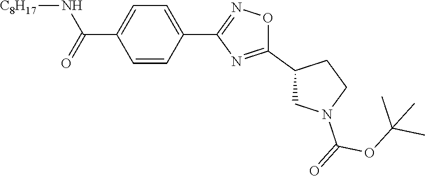

Synthesized according to General Procedure 4. Purified by silica chromatography (60% ethyl acetate in hexanes). White solid (69%, 329 mg). ¹H NMR (400 MHz, Chloroform-d) δ 8.00 (d, J=8.0 Hz, 2H), 7.83 (d, J=8.0 Hz, 2H), 7.03 (t, J=5.7 Hz, 1H), 3.85-3.75 (m, 1H), 3.71-3.50 (m, 3H), 3.47-3.31 (m, 3H), 2.38-2.22 (m, 2H), 1.54 (p, J=7.4 Hz, 2H), 1.40 (s, 9H), 1.33-1.08 (m, 10H), 0.79 (t, J=6.7 Hz, 3H). ¹³C NMR (101 MHz, Chloroform-d) δ 179.72, 167.64, 166.79, 154.16, 137.30, 129.02, 127.55, 127.39, 79.76, 49.03, 45.03, 40.27, 36.05, 31.74, 30.27, 29.56, 29.26, 29.16, 28.42, 27.01, 22.57, 14.04.

### tert-butyl (3-(3-(4-(octylcarbamoyl)phenyl)-1,2,4-oxadiazol-5-yl)propyl)carbamate (12d)

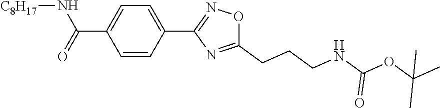

Synthesized according to General Procedure 4. Purified by silica chromatography (60% ethyl acetate in hexanes). White solid (48%, 188 mg). ¹H NMR (400 MHz, Chloroform-d) δ 8.04 (d, J=8.0 Hz, 2H), 7.83 (d, J=8.0 Hz, 2H), 6.68 (t, J=5.6 Hz, 1H), 4.97 (s, 1H), 3.40 (q, J=6.8 Hz, 2H), 3.24 (q, J=6.5 Hz, 2H), 2.95 (t, J=7.4 Hz, 2H), 2.03 (p, J=7.0 Hz, 2H), 1.57 (p, J=7.3 Hz, 2H), 1.39 (s, 9H), 1.36-1.17 (m, 10H), 0.83 (t, J=6.5 Hz, 3H). ¹³C NMR (101 MHz, Chloroform-d) δ 179.68, 167.61, 166.85, 156.08, 137.23, 129.40, 127.52, 127.50, 79.41, 40.34, 39.77, 31.83, 29.66, 29.34, 29.25, 28.43, 27.09, 26.94, 24.08, 22.67, 14.12.

### (R)—N-butyl-4-(5-(pyrrolidin-3-yl)-1,2,4-oxadiazol-3-yl)benzamide hydrochloride (13a)

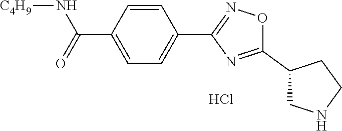

Synthesized according to General Procedure 6. Purified via trituration with diethyl ether. White solid (36%, 102 mg). ¹H NMR (400 MHz, Methanol-d₄) δ 8.11 (d, J=8.4 Hz, 2H), 7.93 (d, J=8.4 Hz, 2H), 3.91 (p, J=8.2, 1H), 3.65-3.58 (m, 1H), 3.53-3.47 (m, 1H), 3.39 (t, J=7.2 Hz, 2H), 3.36-3.26 (m, 2H) 2.49 (h, J=7.9 Hz, 1H), 2.34 (h, J=7.8 Hz, 1H), 1.60 (q, J=7.9 Hz, 2H), 1.41 (h, J=7.3 Hz, 2H), 0.96 (t, J=7.3 Hz, 3H). ¹³C NMR (101 MHz, Methanol-d₄) δ 182.13, 169.06, 168.85, 138.45, 130.56, 128.90, 128.40, 50.86, 47.13, 40.83, 37.41, 32.57, 31.43, 21.19, 14.15. HRMS: (ESI) [M+H]+ calc. for C₁₇H₂₃N₄O₂, 315.1816, observed, 315.18225.

### 4-(5-(3-aminopropyl)-1,2,4-oxadiazol-3-yl)-N-butylbenzamide hydrochloride (13b)

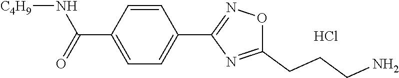

Synthesized according to General Procedure 6. Purified via trituration with diethyl ether. White solid (38%, 80 mg). ¹H NMR (400 MHz, Methanol-d₄) δ 8.16 (d, J=8.4 Hz, 2H), 7.99 (d, J=8.5 Hz, 2H), 3.43 (t, J=7.2 Hz, 2H), 3.20 (t, J=7.5 Hz, 4H), 2.31 (p, J=7.5 Hz, 2H), 1.66 (p, J=7.3 Hz, 2H), 1.46 (h, J=7.3 Hz, 2H), 1.01 (t, J=7.4 Hz, 3H). ¹³C NMR (101 MHz, Methanol-d₄) δ 180.69, 169.12, 168.84, 138.42, 130.69, 128.93, 128.37, 40.83, 39.85, 32.58, 25.11, 24.33, 21.20, 14.15.

### (R)—N-octyl-4-(5-(pyrrolidin-3-yl)-1,2,4-oxadiazol-3-yl)benzamide hydrochloride (13c)

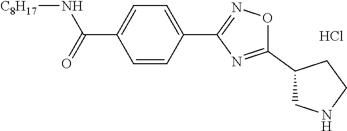

Synthesized according to General Procedure 6. Purified via trituration with diethyl ether. White solid. ¹H NMR (400 MHz, cd₃od) δ 8.61 (t, J=5.7 Hz, 1H), 8.16 (d, J=8.5 Hz, 2H), 7.95 (d, J=8.5 Hz, 2H), 4.19-4.07 (m, 1H), 3.90-3.75 (m, 2H), 3.62-3.46 (m, 2H), 3.44-3.33 (m, 2H), 2.72-2.58 (m, 1H), 2.54-2.41 (m, 1H), 1.64 (p, J=7.7 Hz, 2H), 1.46-1.27 (m, 10H), 0.90 (t, J=7.1 Hz, 3H). ¹³C NMR (101 MHz, cd₃od) δ 180.55, 169.18*, 169.11, 169.02, 138.70*, 138.66, 130.41, 128.96, 128.48, 49.47, 46.62, 41.28, 41.26*, 41.16, 36.68, 32.99, 30.45, 30.43, 30.38, 28.11, 23.71, 14.43.

### 4-(5-(3-aminopropyl)-1,2,4-oxadiazol-3-yl)-N-octylbenzamide hydrochloride (13d)

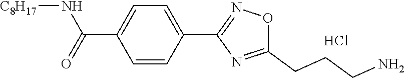

Synthesized according to General Procedure 6. Purified via trituration with diethyl ether. White solid (96%, 155 mg). ¹H NMR (400 MHz, Methanol-d₄) δ 8.15 (d, J=8.4 Hz, 2H), 7.95 (d, J=8.4 Hz, 2H), 3.40 (t, J=7.2 Hz, 2H), 3.19-3.13 (m, 4H), 2.26 (p, J=7.5 Hz, 2H), 1.64 (p, J=7.2 Hz, 2H), 1.44-1.28 (m, 10H), 0.89 (t, J=6.8 Hz, 3H). ¹³C NMR (101 MHz, Methanol-d₄) δ 179.22, 167.71, 167.46, 137.10, 129.25, 127.49, 126.94, 39.71, 38.40, 31.55, 29.01, 28.98, 28.93, 26.67, 23.75, 22.83, 22.26, 12.98. HRMS: (ESI) [M+H]+ calc. for C₂₀H₃₁N₄O₂, 359.2442, observed, 359.2432.

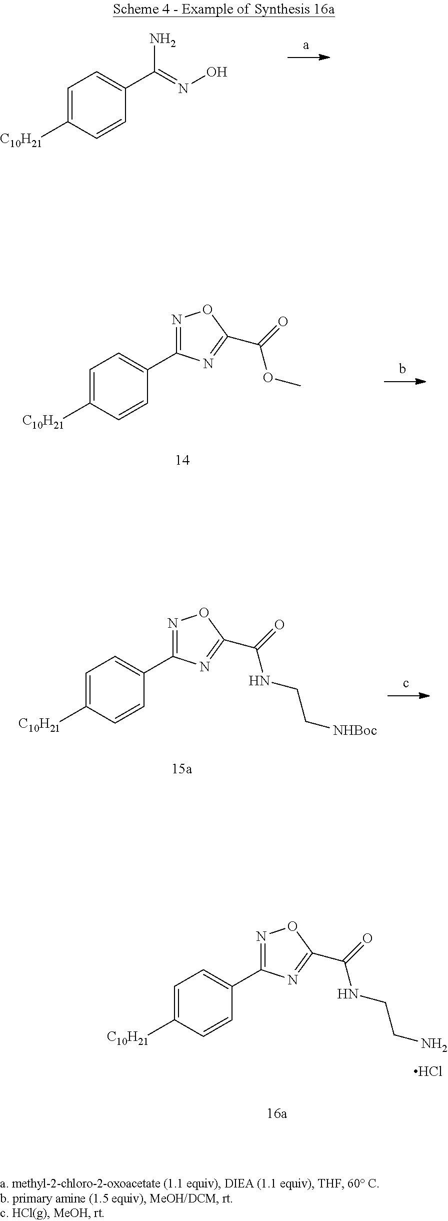

### methyl 3-(4-decylphenyl)-1,2,4-oxadiazole-5-carboxylate (14)

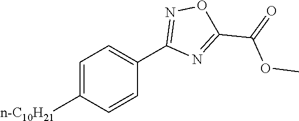

Synthesized according to General Procedure 9 from 3a. Purified on silica gel (10% ethyl acetate/hexanes). White solid (0.79 g, 64%). ¹H NMR (400 MHz, Chloroform-d) δ 8.05 (d, J=8.1 Hz, 2H), 7.31 (d, J=8.1 Hz, 2H), 4.11 (s, 3H), 2.67 (t, J=7.8 Hz, 2H), 1.64 (p, J=7.4 Hz, 2H), 1.38-1.18 (m, 14H), 0.87 (t, J=6.8 Hz, 3H); ¹³C NMR (101 MHz, Chloroform-d) δ 169.68, 166.30, 154.81, 147.58, 129.23, 127.77, 123.04, 54.27, 36.15, 32.04, 31.32, 29.75, 29.71, 29.61, 29.47, 29.42, 22.83, 14.26; HRMS (ESI+): Calcd for C₂₀H₂₉N₂O₃ [M+H]⁺: 345.2173, Found: 345.2170.

### tert-butyl (2-(3-(4-decylphenyl)-1,2,4-oxadiazole-5-carboxamido)ethyl)carbamate (15a)

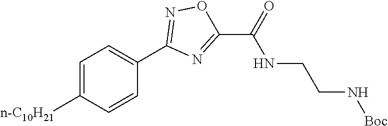

Synthesized according to General Procedure 10 from 14. Purified on silica gel (30% ethyl acetate/hexanes). White solid (0.23 g, 85%). ¹H NMR (400 MHz, Chloroform-d) δ 8.06 (t, J=5.8 Hz, 1H), 7.97 (d, J=8.0 Hz, 2H), 7.27 (d, J=8.2 Hz, 2H), 5.11 (t, J=6.1 Hz, 1H), 3.62 (q, J=5.5 Hz, 2H), 3.43 (q, J=5.9 Hz, 2H), 2.64 (t, J=7.8 Hz, 2H), 1.62 (p, J=7.4 Hz, 2H), 1.42 (s, 9H), 1.37-1.13 (m, 14H), 0.86 (t, J=6.8 Hz, 3H); ¹³C NMR (101 MHz, Chloroform-d) δ 168.89, 168.52, 157.14, 153.89, 147.31, 129.10, 127.60, 123.20, 80.19, 41.50, 39.75, 36.09, 32.00, 31.29, 29.71, 29.68, 29.57, 29.43, 29.39, 28.40, 22.79, 14.23; HRMS (ESI+): Calcd for C₂₁H₃₃N₄O₂ [M+H-Boc]*: 373.2598, Found: 373.2611.

### tert-butyl (3-(3-(4-decylphenyl)-1,2,4-oxadiazole-5-carboxamido)propyl)carbamate (15b)

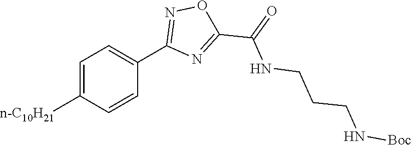

Synthesized according to General Procedure 10 from 14. Purified on silica gel (30% ethyl acetate/hexanes). White solid (0.20 g, 95%). ¹H NMR (400 MHz, Chloroform-d) δ 8.00 (d, J=7.9 Hz, 2H), 7.98-7.93 (m, 1H), 7.29 (d, J=8.1 Hz, 2H), 4.95 (t, J=6.9 Hz, 1H), 3.56 (q, J=6.4 Hz, 2H), 3.25 (q, J=6.3 Hz, 2H), 2.65 (t, J=7.7 Hz, 2H), 1.79 (p, J=6.2 Hz, 2H), 1.63 (p, J=7.0 Hz, 2H), 1.45 (s, 9H), 1.34-1.20 (m, 14H), 0.87 (t, J=6.7 Hz, 3H); ¹³C NMR (101 MHz, Chloroform-d) δ 168.93, 168.69, 156.88, 153.67, 147.31, 129.14, 127.66, 123.28, 79.73, 37.32, 36.85, 36.11, 32.01, 31.32, 30.11, 29.72, 29.69, 29.59, 29.44, 29.39, 28.50, 22.80, 14.24.

### N-(2-aminoethyl)-3-(4-decylphenyl)-1,2,4-oxadiazole-5-carboxamide hydrochloride (16a)

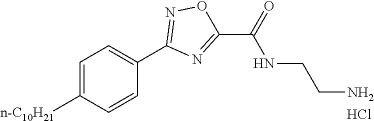

Synthesized according to General Procedure 6. Purified on silica gel (10% methanol/dichloromethane). White solid (0.17 g, 96%). ¹H NMR (400 MHz, Methylene Chloride-d₂) δ 8.00 (d, J=8.0 Hz, 2H), 7.33 (d, J=8.0 Hz, 2H), 3.75 (t, J=5.9 Hz, 2H), 3.21 (t, J=5.9 Hz, 2H), 2.67 (t, J=7.7 Hz, 2H), 1.63 (p, J=7.3 Hz, 2H), 1.45-1.14 (m, 14H), 0.86 (t, J=6.8 Hz, 3H); ¹³C NMR (101 MHz, Methylene Chloride-d₂) δ 169.56, 168.96, 155.43, 148.11, 129.65, 127.86, 123.64, 39.61, 37.80, 36.38, 32.40, 31.74, 30.09, 30.07, 29.95, 29.82, 29.74, 23.16, 14.22; HRMS (ESI+): Calcd for C₂₁H₃₃N₄O₂ [M+H]⁺: 373.2598, Found: 373.2602.

### N-(3-aminopropyl)-3-(4-decylphenyl)-1,2,4-oxadiazole-5-carboxamide hydrochloride (16b)

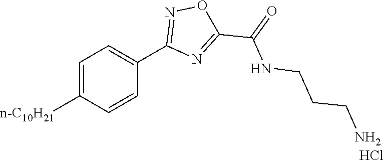

Synthesized according to General Procedure 6. Purified on silica gel (10% methanol/dichloromethane). White solid (0.14 g, 90%). ¹H NMR (400 MHz, Methanol-d₄) δ 8.00 (d, J=7.8 Hz, 2H), 7.34 (d, J=7.9 Hz, 2H), 3.55 (t, J=6.6 Hz, 2H), 3.06 (t, J=7.6 Hz, 2H), 2.67 (t, J=7.7 Hz, 2H), 2.03 (p, J=7.0 Hz, 2H), 1.64 (p, J=7.1 Hz, 2H), 1.42-1.16 (m, 14H), 0.88 (t, J=6.5 Hz, 3H); ¹³C NMR (101 MHz, Methanol-d₄) δ 170.15, 155.86, 148.56, 130.21, 128.47, 124.65, 38.45, 37.71, 36.86, 33.06, 32.41, 30.70, 30.56, 30.45, 30.31, 28.44, 23.73, 14.46; HRMS (ESI+): Calcd for C₂₂H₃₅N₄O₂ [M+H]⁺: 387.2755, Found: 387.2737.

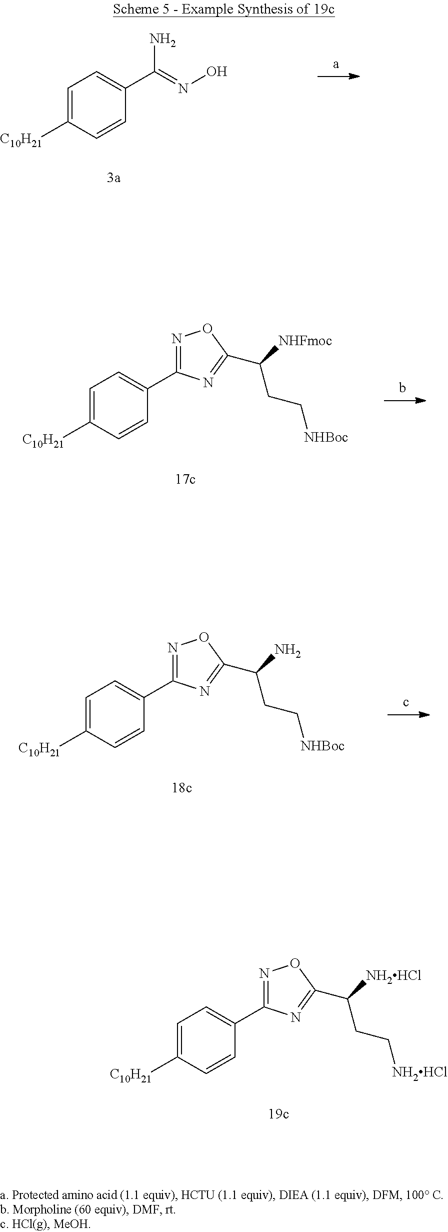

### (9H-fluoren-9-yl)methyl tert-butyl (1-(3-(4-decylphenyl)-1,2,4-oxadiazol-5-yl)pentane-1,5-diyl)(S)-dicarbamate (17a)

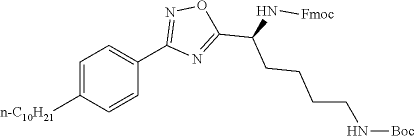

Synthesized according to General Procedure 4. Purified by silica gel (30% ethyl acetate/hexane). White Solid (0.36 g, 73%). ¹H NMR (400 MHz, Chloroform-d) δ 7.97 (d, J=7.9 Hz, 2H), 7.76 (d, J=7.6 Hz, 2H), 7.62 (t, J=7.0 Hz, 2H), 7.40 (t, J=7.5 Hz, 2H), 7.32 (dd, J=7.2, 2.7 Hz, 2H), 7.28 (d, J=8.2 Hz, 2H), 5.71 (d, J=8.6 Hz, 1H), 5.15 (q, J=7.7 Hz, 1H), 4.62 (t, J=6.8 Hz, 1H), 4.49 (dd, J=10.6, 6.9 Hz, 1H), 4.46-4.37 (m, 1H), 4.24 (t, J=7.0 Hz, 1H), 3.13 (q, J=7.2 Hz, 2H), 2.66 (t, J=7.7 Hz, 2H), 2.13-1.89 (m, 2H), 1.65 (p, J=14.8, 7.4 Hz, 2H), 1.58-1.48 (m, 2H), 1.43 (s, 11H), 1.36-1.18 (m, 14H), 0.88 (t, J=6.8 Hz, 3H); ¹³C NMR (101 MHz, Chloroform-d) δ 178.99, 168.41, 156.26, 155.97, 146.82, 143.88, 143.73, 141.41, 129.03, 127.84, 127.58, 127.20, 125.20, 125.16, 123.89, 120.10, 79.37, 67.30, 48.87, 47.26, 40.00, 36.08, 33.62, 32.01, 31.34, 29.72, 29.70, 29.65, 29.59, 29.44, 29.37, 28.52, 22.80, 22.46, 14.25; HRMS (ESI+): Calcd for C₄₃H₅₇N₄O₅ [M+H]⁺: 709.4323, Found: 709.4322.

### (9H-fluoren-9-yl)methyl tert-butyl (1-(3-(4-decylphenyl)-1,2,4-oxadiazol-5-yl)butane-1,4-diyl)(S)-dicarbamate (17b)

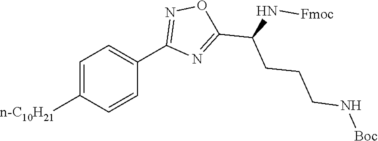

Synthesized according to General Procedure 4. Purified by silica gel (30% ethyl acetate/hexanes). Yellow solid (0.20 g, 40%). ¹H NMR (400 MHz, Chloroform-d) δ 7.97 (d, J=8.0 Hz, 2H), 7.76 (d, J=7.6 Hz, 2H), 7.62 (t, J=7.3 Hz, 2H), 7.40 (t, J=7.5 Hz, 2H), 7.34-7.30 (m, 2H), 7.28 (d, J=8.2 Hz, 2H), 5.85-5.70 (m, 1H), 5.27-5.09 (m, 1H), 4.64 (s, 1H), 4.51-4.42 (m, 2H), 4.24 (t, J=7.0 Hz, 1H), 3.27-3.13 (m, 2H), 2.66 (t, J=7.7 Hz, 2H), 2.14-2.00 (m, 1H), 1.99-1.87 (m, 1H), 1.70-1.62 (m, 2H), 1.61-1.54 (m, 2H), 1.45 (s, 9H), 1.39-1.19 (m, 14H), 0.88 (t, J=6.8 Hz, 3H); ¹³C NMR (101 MHz, Chloroform-d) δ 178.82, 168.46, 156.23, 155.94, 146.88, 143.91, 143.73, 141.46, 129.06, 127.87, 127.60, 127.22, 125.20, 123.87, 120.12, 79.56, 67.27, 48.85, 47.31, 39.91, 36.09, 32.02, 31.33, 31.26, 29.73, 29.70, 29.60, 29.45, 29.38, 28.52, 26.34, 22.81, 14.24.

### (9H-fluoren-9-yl)methyl tert-butyl (1-(3-(4-decylphenyl)-1,2,4-oxadiazol-5-yl)propane-1,3-diyl)(S)-dicarbamate (17c)

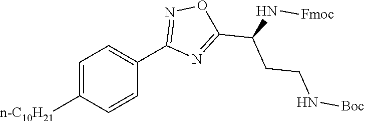

Synthesized according to General procedure 4. Purified by silica gel (25% ethyl acetate/hexanes). White solid (0.36 g, 74%). ¹H NMR (400 MHz, Chloroform-d) δ 7.96 (d, J=7.9 Hz, 2H), 7.77 (d, J=7.6 Hz, 2H), 7.69-7.59 (m, 2H), 7.40 (t, J=7.5 Hz, 2H), 7.33 (dd, J=7.1, 2.7 Hz, 2H), 7.28 (d, J=8.2 Hz, 2H), 6.00 (d, J=9.0 Hz, 1H), 5.24 (q, J=7.9 Hz, 1H), 4.93 (t, J=6.7 Hz, 1H), 4.49 (d, J=7.0 Hz, 2H), 4.26 (t, J=7.0 Hz, 1H), 3.59-3.36 (m, 1H), 3.16-2.98 (m, 1H), 2.66 (t, J=7.7 Hz, 2H), 2.32-2.19 (m, 1H), 2.19-2.02 (m, 1H), 1.64 (p, J=7.2 Hz, 2H), 1.42 (s, 9H), 1.35-1.19 (m, 14H), 0.88 (t, J=6.8 Hz, 3H); ¹³C NMR (101 MHz, Chloroform-d) δ 178.76, 168.47, 156.29, 156.19, 146.88, 143.87, 143.70, 141.46, 129.05, 127.89, 127.60, 127.24, 125.23, 123.86, 120.13, 79.84, 67.39, 47.32, 46.75, 36.47, 36.10, 34.37, 32.03, 31.34, 29.74, 29.71, 29.61, 29.46, 29.38, 28.48, 22.82, 14.25.

### (9H-fluoren-9-yl)methyl tert-butyl (1-(3-(4-decylphenyl)-1,2,4-oxadiazol-5-yl)pentane-1,5-diyl)(R)-dicarbamate (18d)

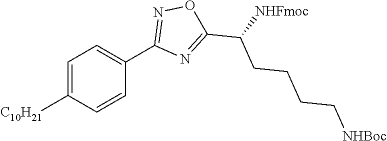

Synthesized according to General Procedure 4. Crude mixture concentrated and carried forward without further purification.

### tert-butyl (S)-(5-amino-5-(3-(4-decylphenyl)-1,2,4-oxadiazol-5-yl)pentyl)carbamate (18a)

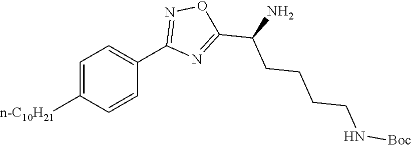

Synthesized according to General Procedure 11. Purified by silica gel (5% methanol/dichloromethane). Yellow oil (0.06 g, 85%). ¹H NMR (400 MHz, Chloroform-d) δ 7.96 (d, J=7.9 Hz, 2H), 7.27 (d, J=8.1 Hz, 2H), 4.58 (s, 1H), 4.19 (t, J=6.8 Hz, 1H), 3.12 (q, J=6.5 Hz, 2H), 2.64 (t, J=7.7 Hz, 2H), 2.01-1.92 (m, 2H), 1.91-1.78 (m, 2H), 1.62 (p, J=7.4 Hz, 2H), 1.58-1.51 (m, 2H), 1.50-1.44 (m, 2H), 1.41 (s, 9H), 1.34-1.20 (m, 14H), 0.86 (t, J=6.7 Hz, 3H); ¹³C NMR (101 MHz, Chloroform-d) δ 182.47, 168.31, 156.06, 146.68, 129.03, 127.51, 124.14, 79.22, 49.32, 40.35, 36.07, 35.66, 32.01, 31.35, 29.86, 29.72, 29.69, 29.59, 29.44, 29.36, 28.51, 22.96, 22.80, 14.24; HRMS (ESI+): Calcd for C₂₈H₄₇N₄O₃ [M+H]⁺: 487.3643, Found: 487.3632.

### tert-butyl (S)-(4-amino-4-(3-(4-decylphenyl)-1,2,4-oxadiazol-5-yl)butyl)carbamate (18b)

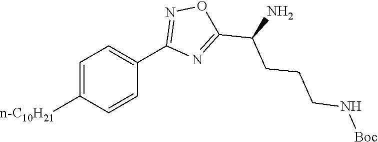

Synthesized according to General Procedure 11. Purified by silica gel (5% methanol/dichloromethane). Yellow oil (0.03 g, 83%). ¹H NMR (400 MHz, Chloroform-d) δ 7.97 (d, J=8.1 Hz, 2H), 7.28 (d, J=8.1 Hz, 2H), 4.69 (s, 1H), 4.24 (t, J=6.9 Hz, 1H), 3.18 (q, J=6.7 Hz, 2H), 2.65 (t, J=7.7 Hz, 2H), 2.22-2.02 (m, OH), 2.01-1.80 (m, 2H), 1.74-1.63 (m, 2H), 1.63-1.57 (m, 2H), 1.43 (s, 9H), 1.36-1.14 (m, 14H), 0.87 (t, J=6.6 Hz, 3H); ¹³C NMR (101 MHz, Chloroform-d) δ 182.25, 168.36, 156.11, 146.75, 129.05, 127.53, 124.12, 79.38, 49.12, 40.19, 36.09, 33.14, 32.03, 31.35, 29.73, 29.71, 29.61, 29.45, 29.39, 28.54, 26.43, 22.81, 14.25; HRMS (ESI+): Calcd for C₂₇H₄₅N₄O₃ [M+H]⁺: 473.3486, Found: 473.3476.

### tert-butyl (S)-(3-amino-3-(3-(4-decylphenyl)-1,2,4-oxadiazol-5-yl)propyl)carbamate (18c)

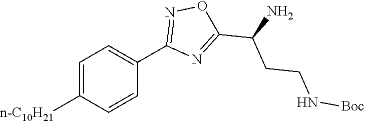

Synthesized according to General Procedure 11. Purified by silica gel (5% methanol/dichloromethane). Yellow oil (0.02 g, 34%). ¹H NMR (400 MHz, Chloroform-d) δ 7.96 (d, J=8.0 Hz, 2H), 7.28 (d, J=8.1 Hz, 2H), 5.06 (s, 1H), 4.28 (dd, J=8.7, 5.0 Hz, 1H), 3.55-3.39 (m, 1H), 3.38-3.20 (m, 1H), 2.65 (t, J=7.7 Hz, 2H), 2.28-2.11 (m, 2H), 2.11-1.90 (m, 2H), 1.63 (p, J=7.2 Hz, 2H), 1.42 (s, 9H), 1.34-1.13 (m, 14H), 0.87 (t, J=6.5 Hz, 3H); ¹³C NMR (101 MHz, Chloroform-d) δ 182.09, 168.39, 156.18, 146.77, 129.06, 127.54, 124.09, 79.55, 47.71, 37.57, 36.10, 35.84, 32.03, 31.35, 29.74, 29.71, 29.61, 29.46, 29.39, 28.52, 22.82, 14.25; HRMS (ESI+): Calcd for C₂₆H₄₃N₄O₃ [M+H]⁺: 459.3330, Found: 459.3325.

### tert-butyl (R)-(5-amino-5-(3-(4-decylphenyl)-1,2,4-oxadiazol-5-yl)pentyl)carbamate (18d)

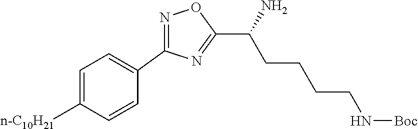

Synthesized according to General Procedure 11. Purified by silica gel (50% ethyl acetate/hexanes). Yellow solid (0.12 g, 44%). ¹H NMR (400 MHz, Chloroform-d) δ 7.96 (d, J=8.2 Hz, 2H), 7.27 (d, J=8.1 Hz, 2H), 4.59 (s, 1H), 4.19 (t, J=6.8 Hz, 1H), 3.11 (q, J=6.4 Hz, 2H), 2.64 (t, J=7.7 Hz, 2H), 2.05-1.85 (m, 2H), 1.83 (s, 2H), 1.62 (p, J=7.4 Hz, 2H), 1.58-1.50 (m, 2H), 1.50-1.43 (m, 2H), 1.41 (s, 9H), 1.33-1.16 (m, 14H), 0.86 (t, J=6.6 Hz, 3H); ¹³C NMR (101 MHz, Chloroform-d) δ 182.48, 168.32, 156.07, 146.68, 129.02, 127.51, 124.16, 79.20, 49.32, 40.35, 36.07, 35.67, 32.00, 31.33, 29.87, 29.71, 29.68, 29.58, 29.43, 29.36, 28.52, 22.96, 22.79, 14.23; HRMS (ESI+): Calcd for C₂₈H₄₇N₄O₃ [M+H]⁺: 487.3643, Found: 487.3634.

### (S)-1-(3-(4-decylphenyl)-1,2,4-oxadiazol-5-yl)pentane-1,5-diamine hydrochloride (19a)

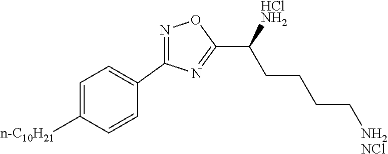

Synthesized according to General Procedure 6. Purified by trituration with diethyl ether. White solid (0.02 g, 37%). ¹H NMR (400 MHz, Methanol-d₄) δ 7.97 (d, J=8.1 Hz, 2H), 7.34 (d, J=8.1 Hz, 2H), 4.29 (t, J=6.8 Hz, 1H), 3.01-2.89 (m, 2H), 2.68 (t, J=7.7 Hz, 2H), 2.09-1.87 (m, 2H), 1.78-1.65 (m, 2H), 1.70-1.59 (m, 2H), 1.62-1.41 (m, 2H), 1.39-1.19 (m, 14H), 0.89 (t, J=6.8 Hz, 3H); ¹³C NMR (101 MHz, Methanol-d₄) δ 183.10, 169.43, 148.11, 130.12, 128.34, 125.35, 49.72, 40.45, 36.84, 35.77, 33.07, 32.47, 30.71, 30.57, 30.46, 30.31, 28.22, 23.74, 23.52, 14.45; HRMS (ESI+): Calcd for C₂₃H₃₉N₄O [M+H]⁺: 387.3118, Found: 387.3124.

### (S)-1-(3-(4-decylphenyl)-1,2,4-oxadiazol-5-yl)butane-1,4-diamine dihydrochloride (19b)

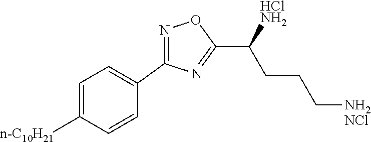

Synthesized according to General Procedure 6. Purified by silica gel (0%-20% methanol/dichloromethane). White solid (0.01 g, 57%). ¹H NMR (400 MHz, Methanol-d₄) δ 7.98 (d, J=8.0 Hz, 2H), 7.35 (d, J=8.0 Hz, 2H), 4.54 (t, J=6.8 Hz, 1H), 3.02 (t, J=7.4 Hz, 2H), 2.69 (t, J=7.7 Hz, 2H), 2.24-1.99 (m, 2H), 1.94-1.75 (m, 2H), 1.65 (p, J=7.3 Hz, 2H), 1.42-1.19 (m, 14H), 0.89 (t, J=6.8 Hz, 3H); ¹³C NMR (101 MHz, Methanol-d₄) δ 180.76, 169.60, 148.30, 130.16, 128.40, 125.07, 49.26, 40.24, 36.84, 33.05, 32.42, 32.28, 30.69, 30.55, 30.43, 30.29, 24.85, 23.72, 14.44; HRMS (ESI+): Calcd for C₂₂H₃₇N₄O [M+H]⁺: 373.2962, Found: 373.2955.

### (S)-1-(3-(4-decylphenyl)-1,2,4-oxadiazol-5-yl)propane-1,3-diamine dihydrochloride (19c)

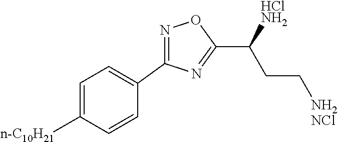

Synthesized according to General Procedure 6. Purified by silica gel (0%-20% methanol/dichloromethane). White solid (0.02 g, 67%). ¹H NMR (400 MHz, Methanol-d₄) δ 7.97 (d, J=8.2 Hz, 2H), 7.34 (d, J=8.1 Hz, 2H), 4.39 (dd, J=8.8, 5.1 Hz, 1H), 3.28-3.15 (m, 2H), 2.68 (t, J=7.7 Hz, 2H), 2.38-2.26 (m, 1H), 2.22-2.08 (m, 1H), 1.66 (p, J=7.5 Hz, 2H), 1.42-1.19 (m, 14H), 0.89 (t, J=6.9 Hz, 3H); ¹³C NMR (101 MHz, Methanol-d₄) δ 182.80, 169.50, 148.14, 130.12, 128.35, 125.31, 48.60, 38.62, 36.84, 33.29, 33.06, 32.44, 30.69, 30.55, 30.44, 30.30, 23.72, 14.43; HRMS (ESI+): Calcd for C₂₁H₃₅N₄O [M+H]⁺: 359.2805, Found: 359.2798.

### (R)-1-(3-(4-decylphenyl)-1,2,4-oxadiazol-5-yl)pentane-1,5-diamine dihydrochloride (19

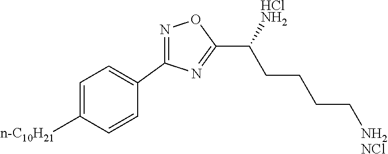

Synthesized according to General Procedure 6. Purified by trituration with diethyl ether. White solid (0.09 g, 79%). ¹H NMR (400 MHz, Methanol-d₄) δ 8.01 (d, J=8.0 Hz, 2H), 7.35 (d, J=8.1 Hz, 2H), 4.98 (t, J=6.9 Hz, 1H), 2.99 (t, J=7.6 Hz, 2H), 2.68 (t, J=7.7 Hz, 2H), 2.34-2.14 (m, 2H), 1.80 (p, J=7.6 Hz, 2H), 1.72-1.63 (m, 2H), 1.63-1.53 (m, 2H), 1.40-1.20 (m, 14H), 0.88 (t, J=6.9 Hz, 3H); ¹³C NMR (101 MHz, Methanol-d₄) δ 176.69, 169.75, 148.48, 130.18, 128.48, 124.60, 48.85, 40.21, 36.83, 33.01, 32.36, 30.66, 30.52, 30.40, 30.27, 27.82, 23.69, 23.05, 14.46; HRMS (ESI+): Calcd for C₂₃H₃₉N₄O [M+H]⁺: 387.3118, Found: 387.3106.

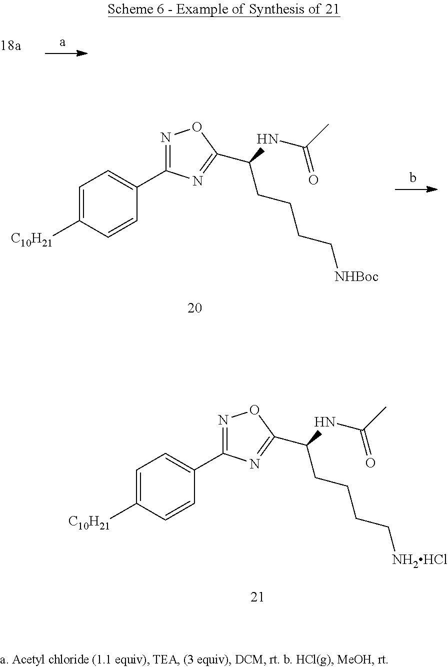

### tert-butyl (S)-(5-acetamido-5-(3-(4-decylphenyl)-1,2,4-oxadiazol-5-yl)pentyl)carbamate (20)

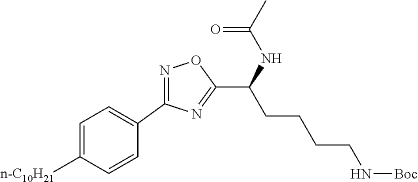

18a (1 equiv) and TEA (3 equiv) were added to a round bottom flask containing DCM. Acetyl chloride (1.1 equiv) was added and the reaction mixture was stirred at room temperature for 4 hours. Concentration in vacuo afforded the crude product, which was then purified by column chromatography using the appropriate ethyl acetate and hexanes solvent system to afford the acetyl protected compound. Purified by silica gel (60% ethyl acetate/hexanes). White solid (0.08 g, 48%). ¹H NMR (400 MHz, Chloroform-d) δ 7.93 (d, J=7.9 Hz, 2H), 7.25 (d, J=8.4 Hz, 2H), 6.71 (d, J=8.1 Hz, 1H), 5.36 (q, J=7.3 Hz, 1H), 4.67 (t, J=6.2 Hz, 1H), 3.09 (q, J=6.5 Hz, 2H), 2.63 (t, J=7.7 Hz, 2H), 2.07 (s, 3H), 2.04-1.84 (m, 2H), 1.61 (p, J=7.3 Hz, 2H), 1.54-1.46 (m, 2H), 1.41 (s, 9H), 1.39-1.33 (m, 2H), 1.33-1.20 (m, 14H), 0.86 (t, J=6.8 Hz, 3H); ¹³C NMR (101 MHz, Chloroform-d) δ 178.97, 170.16, 168.35, 156.33, 146.75, 128.99, 127.51, 123.89, 79.27, 46.83, 39.86, 36.04, 33.28, 31.98, 31.31, 29.69, 29.67, 29.56, 29.41, 29.34, 28.49, 23.13, 22.77, 22.40, 14.22.

### (S)—N-(5-amino-1-(3-(4-decylphenyl)-1,2,4-oxadiazol-5-yl)pentyl)acetamide hydrochloride (21)

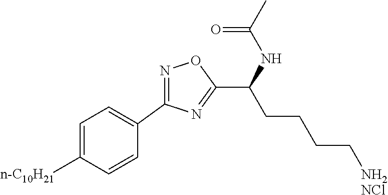

Synthesized according to General Procedure 6. Purified by silica gel (0%-20% methanol/dichloromethane). White solid (0.06 g, 85%). ¹H NMR (400 MHz, Methanol-d₄) δ 7.95 (d, J=8.2 Hz, 2H), 7.33 (d, J=8.1 Hz, 2H), 5.28 (dd, J=8.8, 5.9 Hz, 1H), 2.95 (t, J=7.6 Hz, 2H), 2.67 (t, J=7.7 Hz, 2H), 2.17-2.07 (m, 1H), 2.05 (s, 3H), 2.04-1.94 (m, 1H), 1.80-1.70 (m, 2H), 1.69-1.60 (m, 2H), 1.60-1.46 (m, 2H), 1.38-1.17 (m, 14H), 0.89 (t, J=6.9 Hz, 3H); ¹³C NMR (101 MHz, Methanol-d₄) δ 180.61, 173.38, 169.55, 148.11, 130.10, 128.33, 125.24, 47.76, 40.44, 36.84, 33.30, 33.06, 32.44, 30.70, 30.56, 30.45, 30.30, 27.94, 23.74, 23.68, 22.37, 14.46; HRMS (ESI+): Calcd for C₂₅H₄₁N₄O₂ [M+H]⁺: 429.3224, Found: 429.3225.

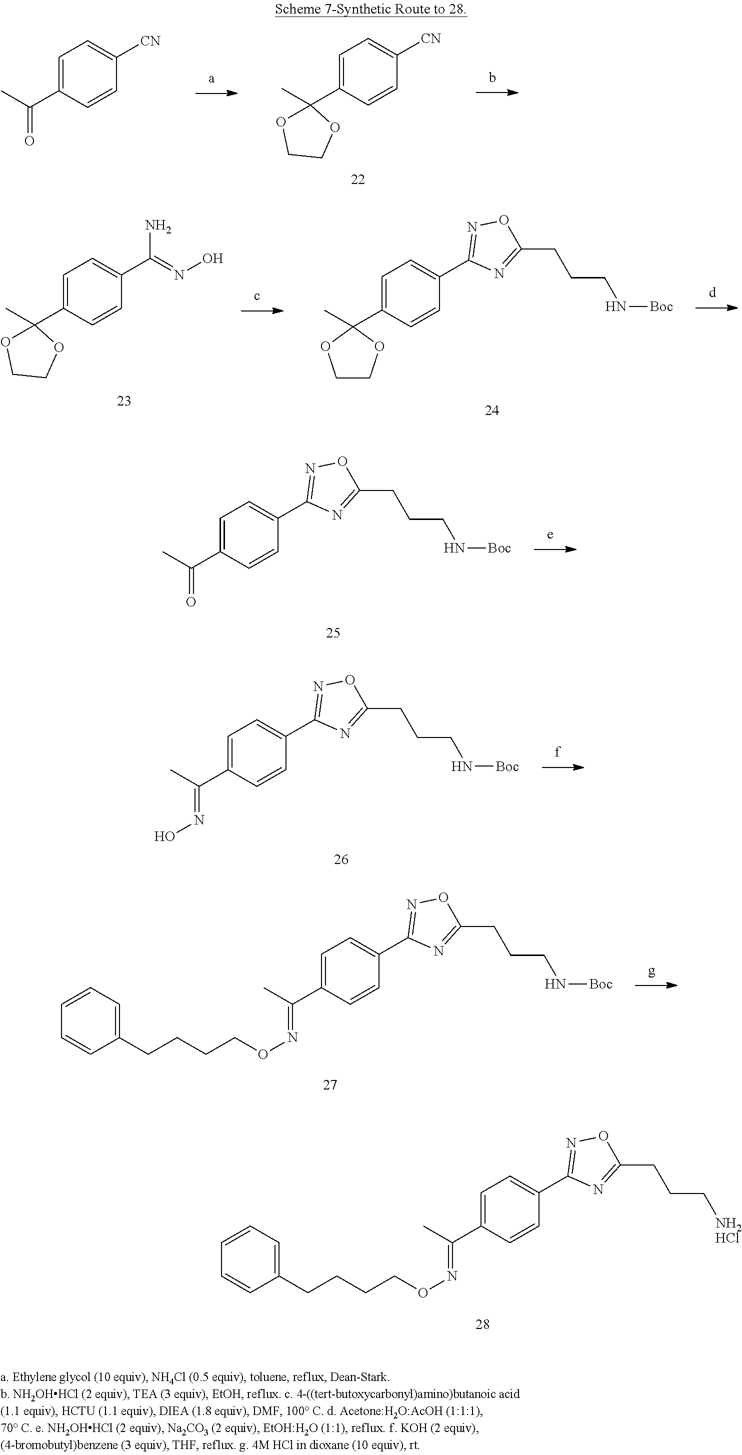

### 4-(2-methyl-1,3-dioxolan-2-yl)benzonitrile (22)

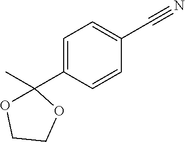

4-acetylbenzonitrile (2.00 g, 12.78 mmol), ethylene glycol (7.68 mL, 137.8 mmol), and ammonium chloride (0.37 g, 6.89 mmol) were added to a round bottom flask containing toluene with Dean-Stark apparatus attached. The mixture was heated to reflux overnight (16-20 hours). Upon cooling to room temperature, excess potassium carbonate was added to quench acid, followed by filtration. Concentration in vacuo afforded the crude product, which was then purified on silica gel (10% ethyl acetate in hexanes) to afford 22 (2.4393 g, 94%) as a white solid. ¹H NMR (400 MHz, Chloroform-d) δ 7.60 (q, J=8.0 Hz, 4H), 4.14-3.95 (m, 2H), 3.77-3.69 (m, 2H), 1.61 (s, 3H); ¹³C NMR (101 MHz, Chloroform-d) δ 148.77, 132.25, 126.23, 118.82, 111.85, 108.22, 64.73, 27.45.

### N′-hydroxy-4-(2-methyl-1,3-dioxolan-2-yl)benzimidamide (23)

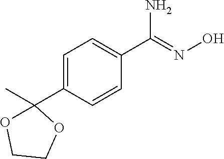 [SMILES: CC1(c2ccc(/C(N)=N/O)cc2)OCCO1]

To a round bottom flask containing ethanol was added 4-(2-methyl-1,3-dioxolan-2-yl)benzonitrile (2.44 g, 12.90 mmol), hydroxylamine hydrochloride (1.79 g, 25.79 mmol), and triethylamine (8.99 mL, 64.48 mmol) under ambient air. The reaction mixture was then heated to reflux for 4 hours. The resulting solution was allowed to cool to room temperature, followed by concentration in vacuo, to afford the crude mixture as a solid. Purification on silica gel (60% ethyl acetate in hexanes) afforded 23 (2.59 g, 91%) as a white solid. ¹H NMR (400 MHz, Methanol-d₄) δ 7.62 (d, J=8.1 Hz, 2H), 7.49 (d, J=8.1 Hz, 2H), 4.88 (s, 2H), 4.15-3.89 (m, 2H), 3.81-3.62 (m, 2H), 1.59 (s, 3H); ¹³C NMR (101 MHz, Methanol-d₄) δ 155.14, 146.29, 133.71, 127.14, 126.42, 109.69, 65.52, 27.82.

### tert-butyl (3-(3-(4-(2-methyl-1,3-dioxolan-2-yl)phenyl)-1,2,4-oxadiazol-5-yl)propyl)carbamate (24)

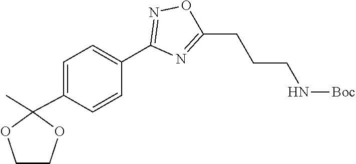 [SMILES: CC(C)(C)OC(=O)NCCCc1nc(-c2ccc(C3(C)OCCO3)cc2)no1]

N′-hydroxy-4-(2-methyl-1,3-dioxolan-2-yl)benzimidamide (0.50 g, 2.25 mmol), 4-((tert-butoxycarbonyl)amino)butanoic acid (23) (0.50 g, 2.48 mmol), and DIEA (2.40 mL, 13.50 mmol) were added to a round bottom flask containing DMF at room temperature. HCTU (1.40 g, 3.38 mmol) was then added and the resulting mixture was heated to 100° C. for 4 hours. Upon cooling to room temperature, the reaction mixture was diluted in ethyl acetate and washed with a saturated lithium bromide solution. The resulting aqueous layer was then extracted with ethyl acetate. The organic layers were then combined and washed with a brine solution, followed by drying over anhydrous sodium sulfate. Concentration in vacuo afforded the crude product, which was then purified by silica gel (30% ethyl acetate/hexanes). Yellow oil (0.76 g, 86%). ¹H NMR (400 MHz, Chloroform-d) δ 7.99 (d, J=8.3 Hz, 2H), 7.55 (d, J=8.2 Hz, 2H), 4.97 (s, 1H), 4.10-3.91 (m, 2H), 3.85-3.66 (m, 2H), 3.24 (q, J=6.5 Hz, 2H), 2.96 (t, J=7.5 Hz, 2H), 2.03 (p, J=7.1 Hz, 2H), 1.62 (s, 3H), 1.39 (s, 9H); ¹³C NMR (101 MHz, Chloroform-d) δ 179.40, 168.03, 156.03, 146.52, 127.39, 126.38, 125.87, 108.59, 79.35, 64.56, 39.78, 28.42, 27.48, 26.95, 24.10; HRMS (ESI+): Calcd for C₂₀H₂₈N₃O₅[M+H]⁺: 390.2023, Found: 390.2023.

### tert-butyl (3-(3-(4-acetylphenyl)-1,2,4-oxadiazol-5-yl)propyl)carbamate (25)

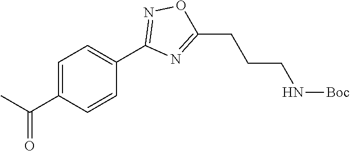 [SMILES: CC(=O)c1ccc(-c2noc(CCCNC(=O)OC(C)(C)C)n2)cc1]

To a round bottom flask was added tert-butyl (3-(3-(4-(2-methyl-1,3-dioxolan-2-yl)phenyl)-1,2,4-oxadiazol-5-yl)propyl)carbamate (24) (0.75 g, 1.93 mmol), followed by a 1:1:1 solution of AcOH:H₂O:acetone. The mixture was heated to 70° C. for 3 hours. Upon cooling to room temperature, the solution was washed with saturated sodium bicarbonate and extracted with ethyl acetate. The organic layers were then combined and washed with a brine solution, followed by drying over anhydrous sodium sulfate. Concentration in vacuo afforded the crude product, which was then purified by silica gel (30% ethyl acetate/hexanes). White solid (0.42 g, 63%). ¹H NMR (400 MHz, Chloroform-d) δ 8.14 (d, J=8.3 Hz, 2H), 8.02 (d, J=8.4 Hz, 2H), 4.86 (t, J=6.2 Hz, 1H), 3.27 (q, J=6.5 Hz, 2H), 3.00 (t, J=7.5 Hz, 2H), 2.62 (s, 3H), 2.07 (p, J=7.0 Hz, 2H), 1.41 (s, 9H); ¹³C NMR (101 MHz, Chloroform-d) δ 197.52, 179.89, 167.60, 156.06, 138.98, 131.03, 128.83, 127.69, 79.51, 39.81, 28.47, 27.02, 26.85, 24.15; HRMS (ESI+): Calcd for C₁₈H₂₄N₃O₄ [M+H]⁺: 346.1761, Found: 346.1760.

### tert-butyl (3-(3-(4-(1-(hydroxyimino)ethyl)phenyl)-1,2,4-oxadiazol-5-yl)propyl)carbamate (26)

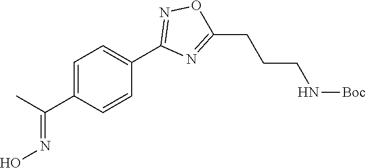 [SMILES: C/C(=N\O)c1ccc(-c2noc(CCCNC(=O)OC(C)(C)C)n2)cc1]

tert-butyl (3-(3-(4-acetylphenyl)-1,2,4-oxadiazol-5-yl)propyl)carbamate (25) (0.42 g, 1.21 mmol), hydroxylamine hydrochloride (0.17 g, 2.41 mmol), and sodium carbonate (0.26 g, 2.41 mmol) were added to a round bottom flask, followed by a 1:1 solution of EtOH:H₂O. The mixture was then refluxed for 16 hours. Upon cooling to room temperature, the mixture was filtered and concentrated in vacuo. The solution was extracted with ethyl acetate. The organic layers were then combined and washed with a brine solution, followed by drying over anhydrous sodium sulfate. Concentration in vacuo afforded the crude product, which was then purified by silica gel (30% ethyl acetate/hexanes). White solid (0.31 g, 72%). ¹H NMR (400 MHz, Chloroform-d) δ 9.67 (s, 1H), 8.04 (d, J=8.1 Hz, 2H), 7.71 (d, J=8.2 Hz, 2H), 4.97 (s, 1H), 3.32-3.24 (m, 2H), 2.98 (t, J=7.5 Hz, 2H), 2.30 (s, 3H), 2.15-1.98 (m, 2H), 1.43 (s, 9H); ¹³C NMR (101 MHz, Chloroform-d) δ 179.60, 167.84, 156.19, 155.07, 139.34, 127.57, 127.26, 126.45, 79.69, 39.82, 28.48, 26.94, 24.12, 12.15; HRMS (ESI+): Calcd for C₁₈H₂₅N₄O₄ [M+H]⁺: 361.1870, Found: 361.1870.

### tert-butyl (3-(3-(4-(1-((4-phenylbutoxy)imino)ethyl)phenyl)-1,2,4-oxadiazol-5-yl)propyl)carbamate (27)

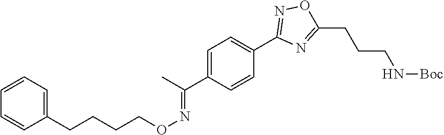 [SMILES: C/C(=N\OCCCCc1ccccc1)c1ccc(-c2noc(CCCNC(=O)OC(C)(C)C)n2)cc1]

tert-butyl (3-(3-(4-(1-(hydroxyimino)ethyl)phenyl)-1,2,4-oxadiazol-5-yl)propyl)carbamate (26) (0.10 g, 0.28 mmol) was added to a round bottom flask containing EtOH. To this solution was added KOH (0.03 g, 0.55 mmol) and stirred for 30 minutes at room temperature. (4-bromobutyl)benzene (0.18 g, 0.83 mmol) was then added to the solution and the mixture was heated to 90° C. for 16 hours. Upon cooling to room temperature, the mixture was filtered and concentrated in vacuo. The solution was extracted with ethyl acetate. The organic layers were then combined and washed with a brine solution, followed by drying over anhydrous sodium sulfate. Concentration in vacuo afforded the crude product, which was then purified by silica gel (20% ethyl acetate/hexanes). Yellow oil (0.06 g, 46%). ¹H NMR (400 MHz, Chloroform-d) δ 8.06 (d, J=8.2 Hz, 2H), 7.76 (d, J=8.2 Hz, 2H), 7.33-7.24 (m, 2H), 7.23-7.14 (m, 3H), 4.90-4.77 (m, 1H), 4.25 (t, J=6.0 Hz, 2H), 3.29 (q, J=6.6 Hz, 2H), 3.00 (t, J=7.5 Hz, 2H), 2.69 (t, J=7.1 Hz, 2H), 2.25 (s, 3H), 2.08 (p, J=7.0 Hz, 2H), 1.83-1.78 (m, 2H), 1.77-1.73 (m, 2H), 1.44 (s, 9H); ¹³C NMR (101 MHz, Chloroform-d) δ 179.49, 168.01, 156.05, 153.49, 142.47, 139.46, 128.51, 128.38, 127.47, 127.12, 126.45, 125.82, 79.50, 74.33, 39.84, 35.75, 28.95, 28.48, 27.92, 27.04, 24.16, 12.60; HRMS (ESI+): Calcd for C₂₈H₃₇N₄O₄ [M+H]⁺: 493.2809, Found: 493.2811.

### 1-(4-(5-(3-aminopropyl)-1,2,4-oxadiazol-3-yl)phenyl)ethan-1-one 0-(4-phenylbutyl) oxime hydrochloride (28)

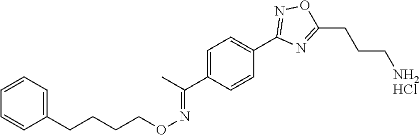

To a 6-dram vial containing tert-butyl (3-(3-(4-(1-((4-phenylbutoxy)imino)ethyl)phenyl)-1,2,4-oxadiazol-5-yl)propyl)carbamate (27) (0.06 g, 0.12 mmol) was added hydrogen chloride (0.3 mL, 1.22 mmol, 4M in dioxane). The resulting mixture was allowed to stir at room temperature for 16 hours. The resulting solution was concentrated in vacuo, washed 3 times with diethyl ether, and then purified by silica gel (0-20% methanol/dichloromethane). White solid (0.03 g, 55%). ¹H NMR (400 MHz, Methanol-d₄) δ 8.03 (d, J=8.5 Hz, 2H), 7.79 (d, J=8.5 Hz, 2H), 7.27-7.20 (m, 2H), 7.20-7.07 (m, 3H), 4.38-3.92 (m, 2H), 3.08 (t, J=7.5 Hz, 2H), 2.95 (t, J=7.4 Hz, 2H), 2.70-2.59 (m, 2H), 2.23 (s, 3H), 2.13 (p, J=7.5 Hz, 2H), 1.79-1.74 (m, 2H), 1.74-1.69 (m, 2H); ¹³C NMR (101 MHz, Methanol-d₄) δ 181.05, 169.08, 154.65, 143.62, 140.76, 129.42, 129.29, 128.34, 128.26, 127.54, 126.73, 75.16, 40.83, 36.59, 29.89, 29.07, 28.00, 24.55, 12.44; HRMS (ESI+): Calcd for C₂₃H₂₉N₄O₂ [M+H]⁺: 393.2285, Found: 393.2275.

SP Transporter Assay

Transporter assays are vectorial and therefore require measurement of the transported analyte in different compartments. The S1P transporter SPNS2 only exports S1P, which obviates measuring uptake of S1P into transporter-expressing cells. Thus, transporter activity was determined by quantifying S1P release from whole cells expressing SPNS2. SPNS2 inhibitor potency was assessed using whole cell assays. HeLa or U937 cells expressing mouse SPNS2 were used to determine inhibitor potency (IC₅₀). Cells were plated onto 12 well plates and assayed when the cell monolayers became confluent. Cell growth media (RPMI-1640 containing 10% fetal bovine serum) was replaced with 2 mL of serum-free media (RPMI-1640) containing fatty acid free bovine serum albumin (BSA) (0.2% w/v) and supplemented with 4-deoxypyridoxine (DOP) (1 mM), NaF (2 mM), Na₃VO₄ (0.2 mM) to inhibit S1P degradation. Test articles (1×10-9-1×10-4 M) were assayed in duplicate or triplicate. After 18 hours, media was collected, an internal recovery standard (0.005 mL of 5×10-7 M deuterated (d7) S1P in methanol) was added, the BSA was precipitated with trichloroacetic acid and the bound S1P extracted with methanol. S1P and S1P-d7 were measured by liquid chromatography mass spectrometry. Inhibitor potency at the human SPNS2 ortholog was determined by an analogous assay using U-937 cells, which endogenously express human SPNS2.

TABLE 2
In vitro inhibition data against SPNS2 for exemplary compounds of
the disclosure (A ≤2 μM, B >2 μM).

| Compound | Structure | IC₅₀ |
|---|---|---|
| 5a | 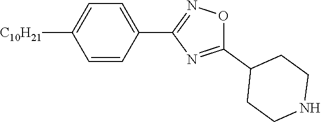 | B |
| 5b | 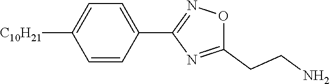 | A |
| 5c | 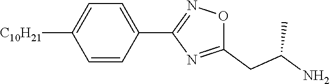 | B |
| 5d | 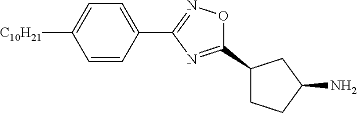 | B |
| 5e | 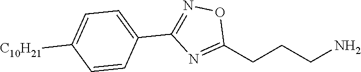 | A |
| 5f | 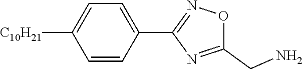 | A |
| 5g | 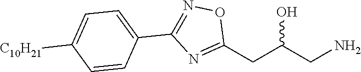 | B |
| 5h | 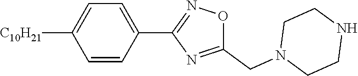 | A |
| 5i | 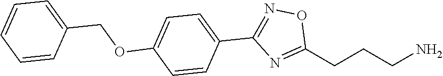 [SMILES: NCCCc1nc(-c2ccc(OCc3ccccc3)cc2)no1] | B |
| 5j | 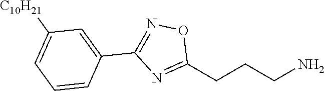 | A |
| 5k | 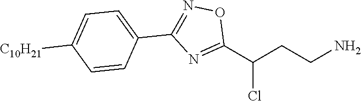 | B |
| 5l | 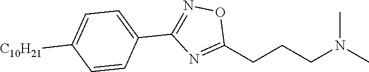 | B |
| 5m | 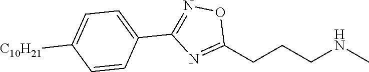 | B |
| 5n | 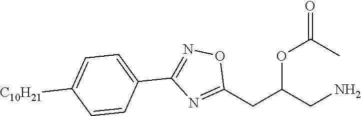 | B |
| 5n | 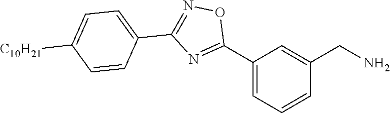 | B |
| 5p | 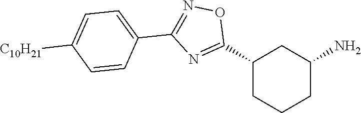 | B |
| 5q | 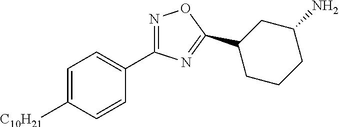 | B |
| 5r | 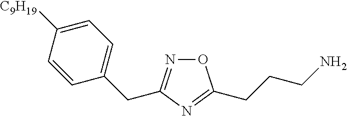 | B |
| 5s | 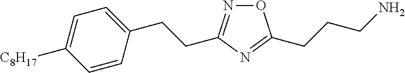 | B |
| 5t | 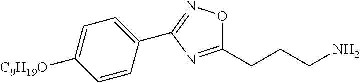 | B |
| 5u | 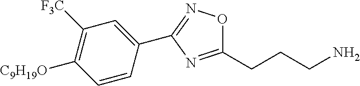 | B |
| 5v | 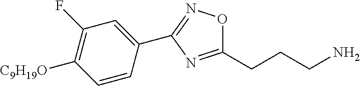 | A |
| 5w | 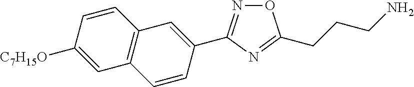 | A |
| 5x | 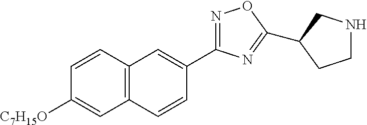 | B |
| 5y | 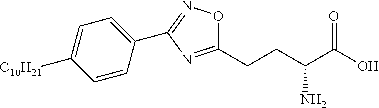 | B |
| 6a | 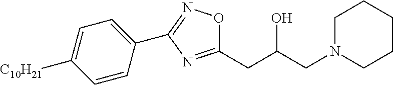 | B |
| 6b | 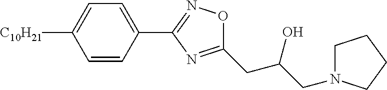 | B |
| 6c | 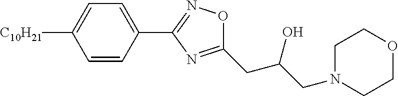 | B |
| 6d | 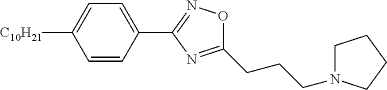 | B |
| 6e | 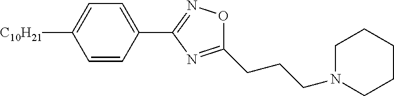 | B |
| 6f | 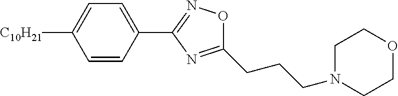 | B |
| 7a | 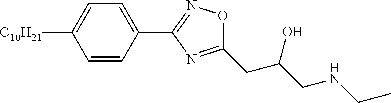 | B |
| 7b | 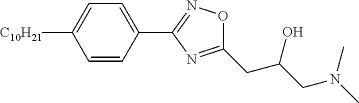 | B |
| 9a | 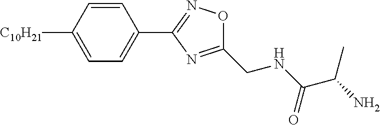 | A |
| 9b | 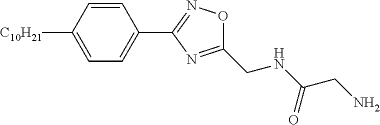 | B |
| 9c | 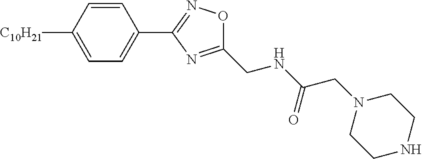 | B |
| 9d | 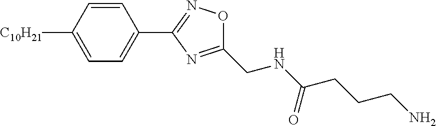 | A |
| 9e | 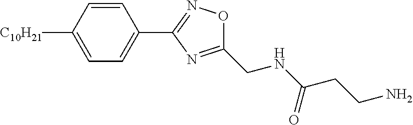 | B |
| 9f | 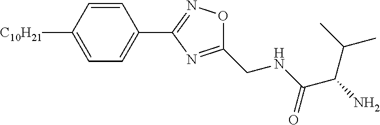 | B |
| 9g | 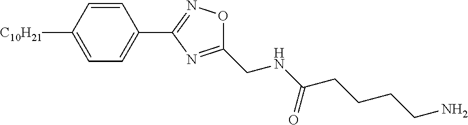 | A |
| 9h | 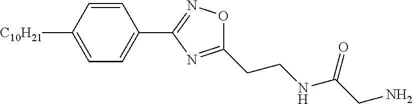 | A |
| 9i | 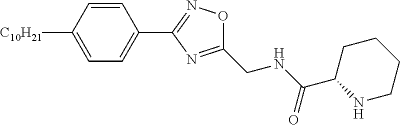 | A |
| 9j | 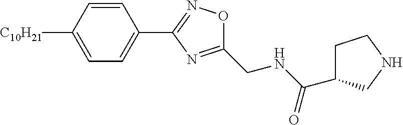 | A |
| 9k | 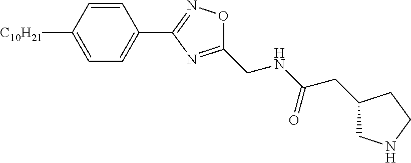 | A |
| 9l | 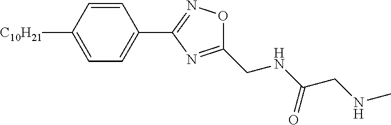 | A |
| 9m | 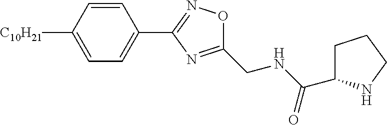 | A |
| 9n | 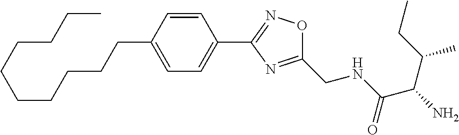 [SMILES: CCCCCCCCCCc1ccc(-c2noc(CNC(=O)[C@@H](N)[C@@H](C)CC)n2)cc1] | B |
| 9o | 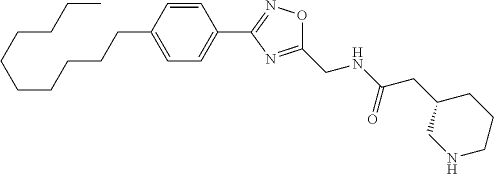 [SMILES: CCCCCCCCCCc1ccc(-c2noc(CNC(=O)C[C@@H]3CCCNC3)n2)cc1] | B |
| 9p | 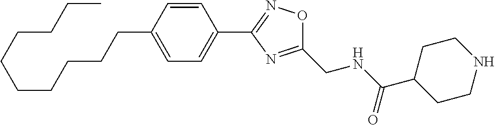 [SMILES: CCCCCCCCCCc1ccc(-c2noc(CNC(=O)C3CCNCC3)n2)cc1] | B |
| 9q | 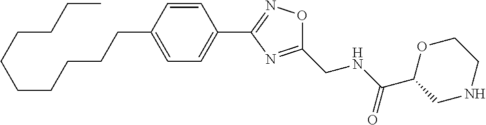 [SMILES: CCCCCCCCCCc1ccc(-c2noc(CNC(=O)[C@H]3CNCCO3)n2)cc1] | B |
| 9r | 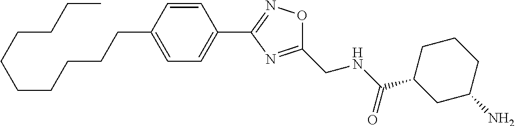 [SMILES: CCCCCCCCCCc1ccc(-c2noc(CNC(=O)[C@@H]3CCC[C@H](N)C3)n2)cc1] | A |
| 9s | 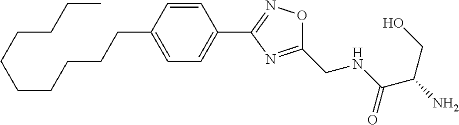 [SMILES: CCCCCCCCCCc1ccc(-c2noc(CNC(=O)[C@@H](N)CO)n2)cc1] | B |
| 9t | 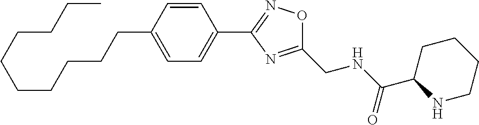 [SMILES: CCCCCCCCCCc1ccc(-c2noc(CNC(=O)[C@H]3CCCCN3)n2)cc1] | B |
| 9u | 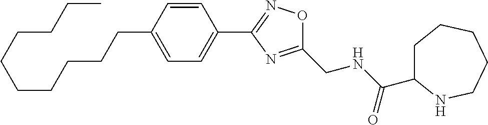 [SMILES: CCCCCCCCCCc1ccc(-c2noc(CNC(=O)C3CCCCCN3)n2)cc1] | A |
| 9v | 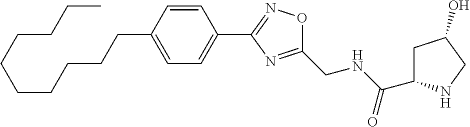 [SMILES: CCCCCCCCCCc1ccc(-c2noc(CNC(=O)[C@@H]3C[C@H](O)CN3)n2)cc1] | B |
| 9w | 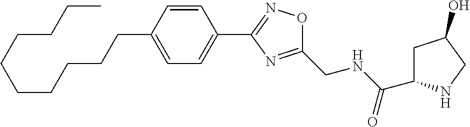 [SMILES: CCCCCCCCCCc1ccc(-c2noc(CNC(=O)[C@@H]3C[C@@H](O)CN3)n2)cc1] | B |
| 9x | 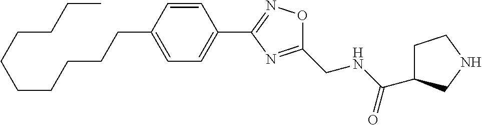 [SMILES: CCCCCCCCCCc1ccc(-c2noc(CNC(=O)[C@H]3CCNC3)n2)cc1] | B |
| 9y | 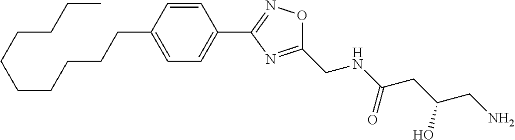 [SMILES: CCCCCCCCCCc1ccc(-c2noc(CNC(=O)C[C@@H](O)CN)n2)cc1] | B |
| 9z | 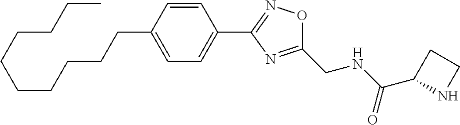 [SMILES: CCCCCCCCCCc1ccc(-c2noc(CNC(=O)[C@@H]3CCN3)n2)cc1] | A |
| 9aa | 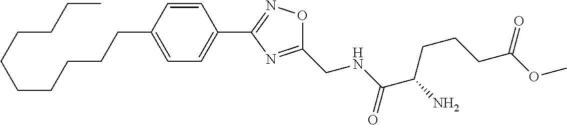 [SMILES: CCCCCCCCCCc1ccc(-c2noc(CNC(=O)[C@@H](N)CCCC(=O)OC)n2)cc1] | A |
| 9ab | 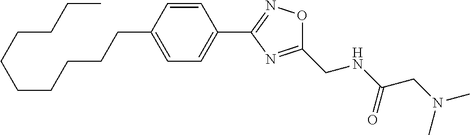 [SMILES: CCCCCCCCCCc1ccc(-c2noc(CNC(=O)CN(C)C)n2)cc1] | B |
| 9ac | 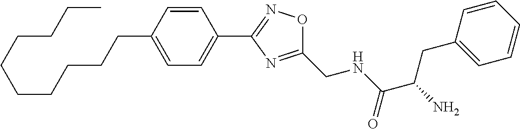 [SMILES: CCCCCCCCCCc1ccc(-c2noc(CNC(=O)[C@@H](N)Cc3ccccc3)n2)cc1] | B |
| 9ad | 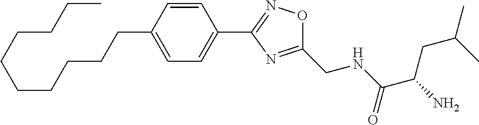 [SMILES: CCCCCCCCCCc1ccc(-c2noc(CNC(=O)[C@@H](N)CC(C)C)n2)cc1] | B |
| 9ae | 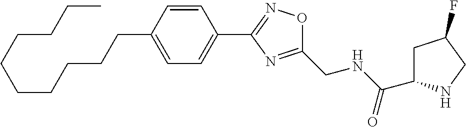 [SMILES: CCCCCCCCCCc1ccc(-c2noc(CNC(=O)[C@@H]3C[C@@H](F)CN3)n2)cc1] | B |
| 9af | 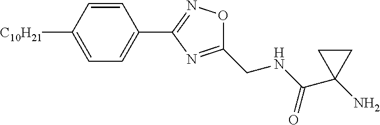 | B |
| 9ag | 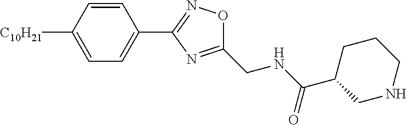 | A |
| 9ah | 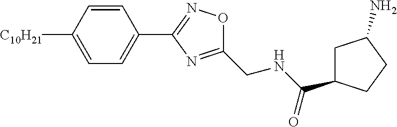 | A |
| 9ai | 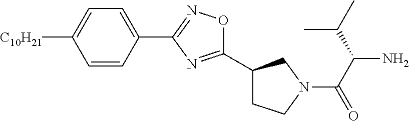 | A |
| 9aj | 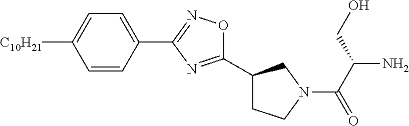 | B |
| 9ak | 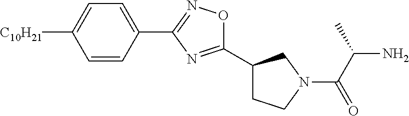 | A |
| 9al | 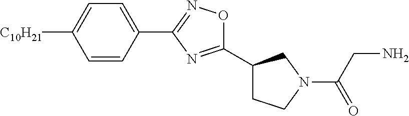 | B |
| 13a | 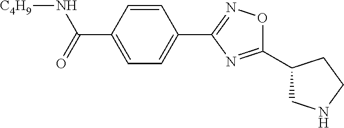 | B |
| 13b | 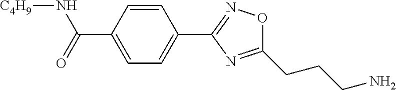 | B |
| 13c | 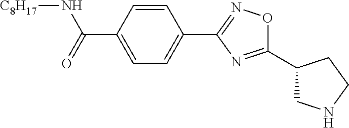 | B |
| 13d | 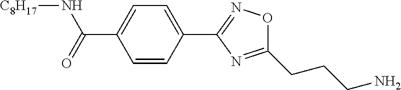 | B |
| 16a | 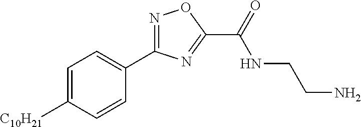 | A |
| 16b | 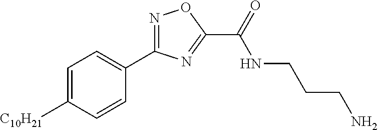 | B |
| 19a | 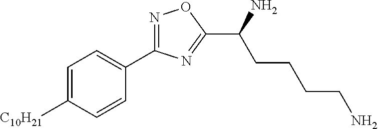 | A |
| 19b | 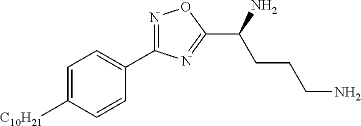 | B |
| 19c | 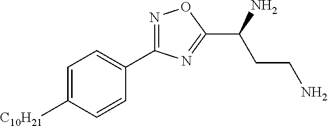 | A |
| 19d | 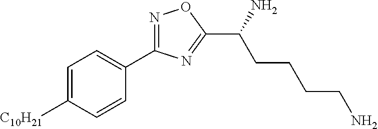 | B |
| 21 | 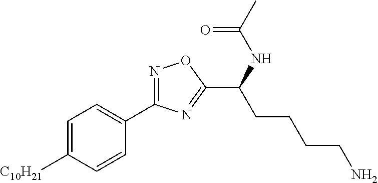 | B |
| 28 | 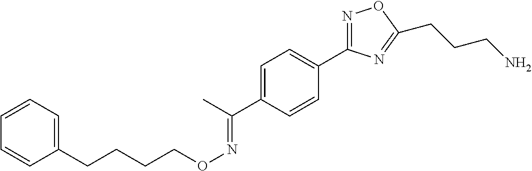 [SMILES: C/C(=N\OCCCCc1ccccc1)c1ccc(-c2noc(CCCN)n2)cc1] | B |

1. A compound according to Formula I: 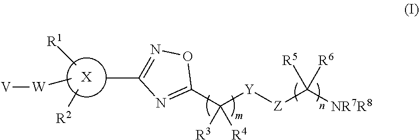
wherein
X is a C₆-C₁₀-aryl or 5- to 10-membered heteroaryl (wherein 1 to 4 heteroaryl ring members are independently selected from N, O, and S);
R¹ and R² are independently selected from the group consisting of H, C₁-C₆-alkyl, C₁-C₆-alkoxy, C₁₋₆₋haloalkoxy, C₃-C₈-cycloalkyl, C₁-C₆-haloalkyl, CN, halo, and —C(O)N(H)(C₁-C₆-alkyl);
W is a bond, O, NH, —NHC(O)—, or —O—(N═)C(R)— (wherein R is H or C₁-C₆-alkyl);
V is selected from the group consisting of H, C₁-C₁₄-alkyl, C₂-C₁₂-alkenyl, (C₆-C₁₀)aryl, (C₆-C₁₀)heteroaryl, —C₁-C₁₀-alkyl-(C₆-C₁₀)aryl, —C₂-C₁₂-alkenyl-(C₆-C₁₀)aryl, —C₁-C₁₀-alkyl-(C₃-C₈)cycloalkyl, -(3- to 14-membered heterocycloalkyl) (wherein 1-4 heterocycloalkyl members are independently selected from N, O, and S), —(C₁-C₁₀)alkyl-(3- to 14-membered heterocycloalkyl) (wherein 1-4 heterocycloalkyl members are independently selected from N, O, and S);
Y is selected from a bond, —NH—, (C₆-C₁₀)arylenyl, and (C₃-C₈)cycloalkylenyl;
Z is selected from a bond and —C(O)—;
R³ and R⁴ are independently selected from the group consisting of, H, C₁-C₆-alkyl, OH, C₁-C₆₋alkoxy, halo, —NRR′, —C(O)R, and —C(O)OR, wherein R and R′ are independently selected from H and C₁-C₆-alkyl;
R⁵ and R⁶ are independently selected from the group consisting of, H, C₁-C₆-alkyl, OH, C₁-C₆₋alkoxy, halo, —C(O)R, and —C(O)OR, wherein R is H or C₁-C₆-alkyl;
or R⁵ and R⁶, together with the carbon to which they are bound, form a —(C₃-C₈)cycloalkyl;
m is an integer selected from 0, 1, 2, 3, 4, 5, and 6;
n is an integer selected from 0, 1, and 2;
R⁷ and R⁸ are independently selected from the group consisting of H and C₁-C₆-alkyl;
or R⁷ and R⁸, together with the nitrogen atom to which they are bound, form a 5- to 7-membered heterocycloalkyl (wherein 1-4 other heterocycloalkyl members are optionally independently selected from NH, 0, and S);
or, optionally, one of R⁵ and R⁶ and one of R⁷ and R⁸, together with the carbon and nitrogen atoms to which they are bound, respectively, form a 4- to 7-membered heterocycloalkyl (wherein 1-4 other heterocycloalkyl members are optionally independently selected from NH, O, and S);
wherein each alkyl, alkoxy, alkenyl, alkynyl, aryl, cycloalkyl, heterocycloalkyl, and heteroaryl is optionally substituted with 1-5 substituents independently selected from the group consisting of hydroxy, halo, C₁-C₆₋haloalkoxy, C₁-C₆-haloalkyl, —NR″₂, —NHC(O)(OC₁-C₆-alkyl), —NO₂, —CN, oxo, —C(O)OH, —C(O)O(C₁-C₆-alkyl), —C₁-C₆-alkyl(C₁-C₆-alkoxy), —C(O)NH₂, C₁-C₆-alkyl, —C(O)C₁-C₆-alkyl, —OC₁-C₆-alkyl, —Si(C₁-C₆-alkyl)₃, —S(O)₀₋₂-(C₁-C₆-alkyl), C₆-C₁₀-aryl, —(C₁-C₆-alkyl)(C₆-C₁₀-aryl), 3- to 14-membered heterocycloalkyl, and —(C₁-C₆-alkyl)-(3- to 14-membered heterocycle) (wherein 1-4 heterocycle members are independently selected from N, O, and S), and —O(C₆-C₁₄-aryl); and
wherein each R″ is independently selected from the group consisting of C₁-C₆-alkyl, C₂-C₆-alkenyl, C₂-C₆-alkynyl, C₆-C₁₀-aryl, 3- to 14-membered heterocycloalkyl and —(C₁-C₆-alkyl)-(3- to 14-membered heterocycloalkyl) (wherein 1-4 ring members are independently selected from N, O, and S), and 5- to 10-membered heteroaryl (wherein 1-4 heteroaryl members are independently selected from N, O, and S;
or a pharmaceutically acceptable salt thereof.

2. The compound or pharmaceutically acceptable salt thereof according to claim 1, wherein, optionally, one of R⁵ and R⁶ and one of R⁷ and R⁸, together with the carbon and nitrogen atoms to which they are bound, respectively, form a 5- to 7-membered heterocycloalkyl (wherein 1-4 other heterocycloalkyl members are optionally independently selected from NH, O, and S).

3. The compound or pharmaceutically acceptable salt thereof according to claim 1, wherein X is phenyl.

4. The compound or pharmaceutically acceptable salt thereof according to claim 3, wherein each of R¹ and R² is H.

5. The compound or pharmaceutically acceptable salt thereof according to claim 4, wherein each of Y and Z is a bond.

6. The compound or pharmaceutically acceptable salt thereof according to claim 5, wherein m is selected from 0, 1, 2, 3, and 4.

7. The compound or pharmaceutically acceptable salt thereof according to claim 6, wherein m is 1, 2, or 3.

8. The compound or pharmaceutically acceptable salt thereof according to claim 7, wherein m is 3 and n is 1 or 2.

9. The compound or pharmaceutically acceptable salt thereof according to claim 8, wherein R⁷ and R⁸ are independently selected from the group consisting of H and C₁-C₆-alkyl.

10. The compound or pharmaceutically acceptable salt thereof claim 9, wherein each of R⁷ and R⁸ is H.

11. The compound or pharmaceutically acceptable salt thereof according to claim 1, wherein Y is (C₃-C₈)cycloalkylenyl and Z is a bond.

12. The compound or pharmaceutically acceptable salt thereof according to claim 11, wherein m is 0 or 1.

13. The compound or pharmaceutically acceptable salt thereof according to claim 12, wherein m is 0.

14. The compound or pharmaceutically acceptable salt thereof according to claim 13, wherein n is 0.

15. The compound or pharmaceutically acceptable salt thereof according claim 4, wherein Y is —NH—.

16. The compound or pharmaceutically acceptable salt thereof according to claim 15, wherein Z is —C(O)—.

17. The compound or pharmaceutically acceptable salt thereof according to claim 16, wherein m is 0 or 1.

18. The compound or pharmaceutically acceptable salt thereof according to claim 17, wherein one of R⁵ and R⁶ and one of R⁷ and R⁸, together with the carbon and nitrogen atoms to which they are bound, respectively, form a 5- to 7-membered heterocycloalkyl.

19. The compound or pharmaceutically acceptable salt thereof according to claim 18, wherein the other one of R⁷ and R⁸ is H.

20. The compound or pharmaceutically acceptable salt thereof according to claim 1, wherein
X is phenyl;
W is a bond and V is C₁-C₁₄-alkyl;
R¹ and R² are independently selected from H and C₁-C₆₋alkyl;
each of Y and Z is a bond;
m is 3, 4, or 5 and n is 0; and
R⁷ and R⁸ are independently selected from H and C₁-C₆₋alkyl.

21. The compound or pharmaceutically acceptable salt thereof according to claim 1, wherein the compound is selected from the following table:

| Compound | Structure |
|---|---|
| 5a | 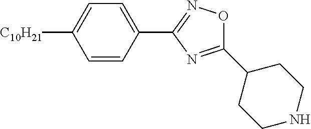 |
| 5b | 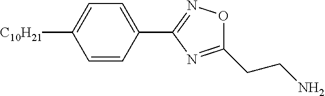 |
| 5c | 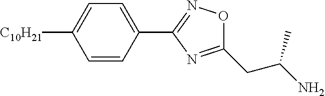 |
| 5d | 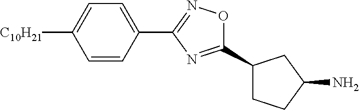 |
| 5e | 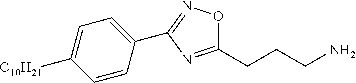 |
| 5f | 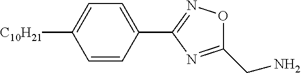 |
| 5g | 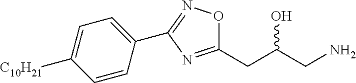 |
| 5h | 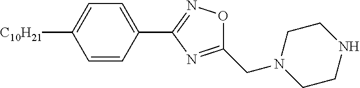 |
| 5i | 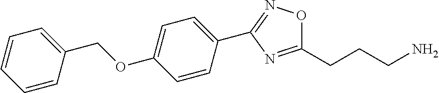 [SMILES: NCCCc1nc(-c2ccc(OCc3ccccc3)cc2)no1] |
| 5g | 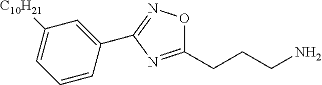 |
| 5k | 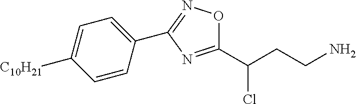 |
| 5l | 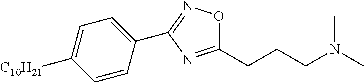 |
| 5m | 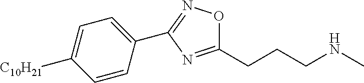 |
| 5n | 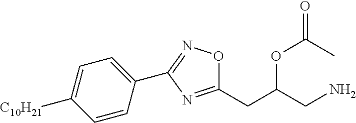 |
| 5n | 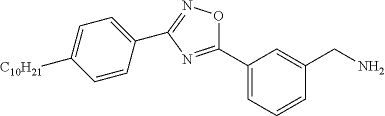 |
| 5p | 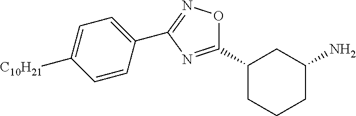 |
| 5q | 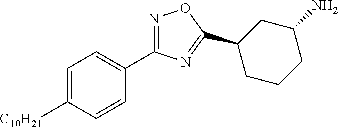 |
| 5r | 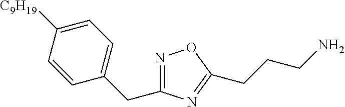 |
| 5s | 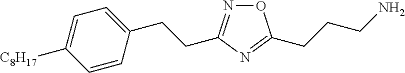 |
| 5t | 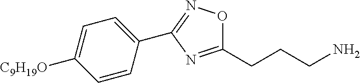 |
| 5u | 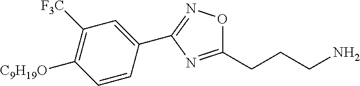 |
| 5v | 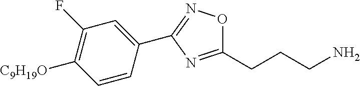 |
| 5w | 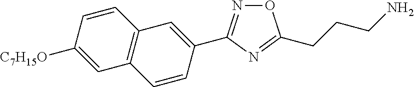 |
| 5x | 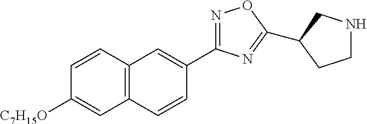 |
| 5y | 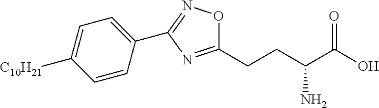 |
| 6a | 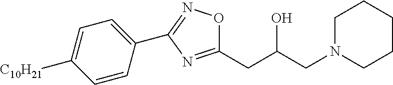 |
| 6b | 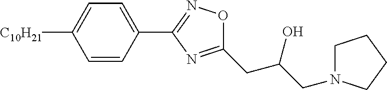 |
| 6c | 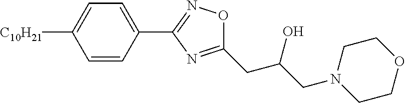 |
| 6d | 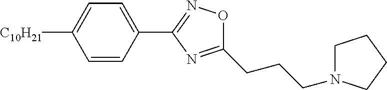 |
| 6e | 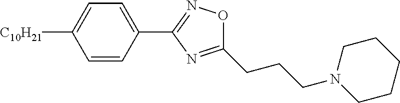 |
| 6f | 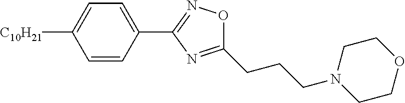 |
| 7a | 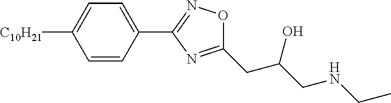 |
| 7b | 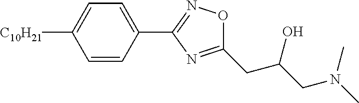 |
| 9a | 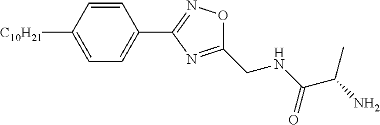 |
| 9b | 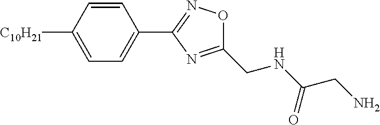 |
| 9c | 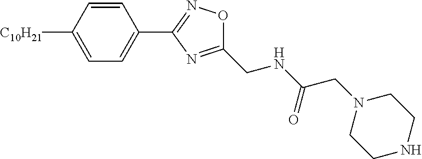 |
| 9d | 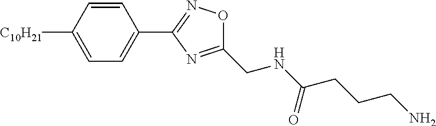 |
| 9e | 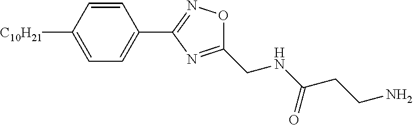 |
| 9f | 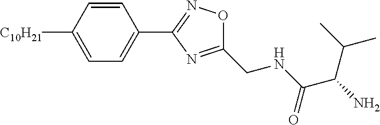 |
| 9g | 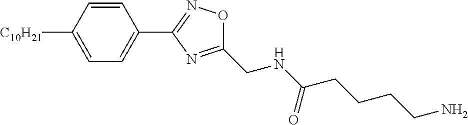 |
| 9h | 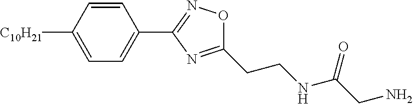 |
| 9i | 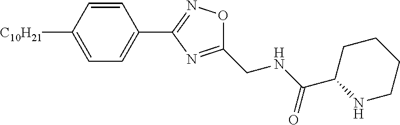 |
| 9j | 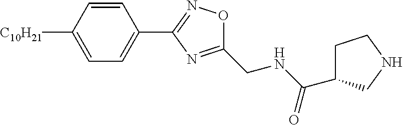 |
| 9k | 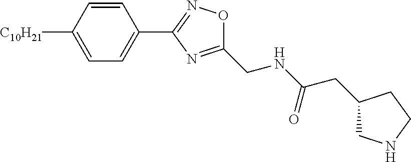 |
| 9l | 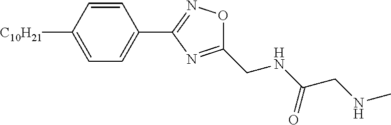 |
| 9m | 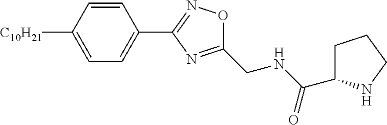 |
| 9n | 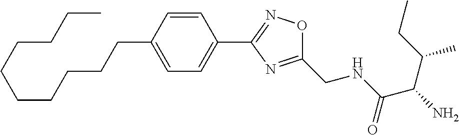 [SMILES: CCCCCCCCCCc1ccc(-c2noc(CNC(=O)[C@@H](N)[C@@H](C)CC)n2)cc1] |
| 9o | 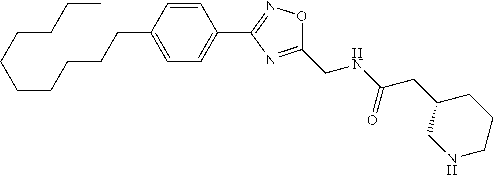 [SMILES: CCCCCCCCCCc1ccc(-c2noc(CNC(=O)C[C@@H]3CCCNC3)n2)cc1] |
| 9p | 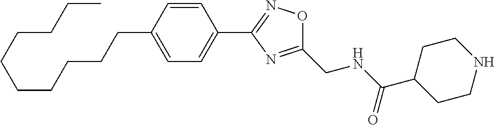 [SMILES: CCCCCCCCCCc1ccc(-c2noc(CNC(=O)C3CCNCC3)n2)cc1] |
| 9q | 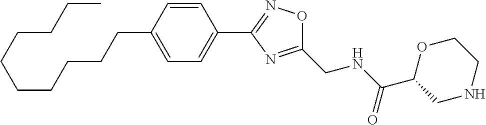 [SMILES: CCCCCCCCCCc1ccc(-c2noc(CNC(=O)[C@H]3CNCCO3)n2)cc1] |
| 9r | 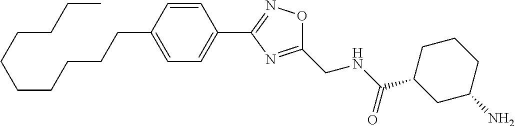 [SMILES: CCCCCCCCCCc1ccc(-c2noc(CNC(=O)[C@@H]3CCC[C@H](N)C3)n2)cc1] |
| 9s | 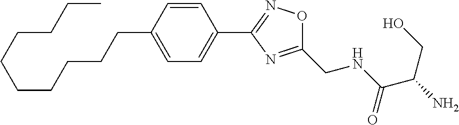 [SMILES: CCCCCCCCCCc1ccc(-c2noc(CNC(=O)[C@@H](N)CO)n2)cc1] |
| 9t | 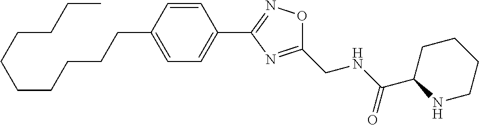 [SMILES: CCCCCCCCCCc1ccc(-c2noc(CNC(=O)[C@H]3CCCCN3)n2)cc1] |
| 9u | 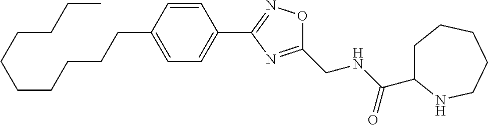 [SMILES: CCCCCCCCCCc1ccc(-c2noc(CNC(=O)C3CCCCCN3)n2)cc1] |
| 9v | 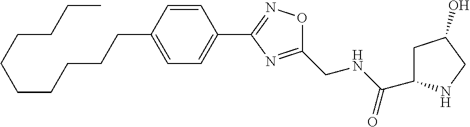 [SMILES: CCCCCCCCCCc1ccc(-c2noc(CNC(=O)[C@@H]3C[C@H](O)CN3)n2)cc1] |
| 9w | 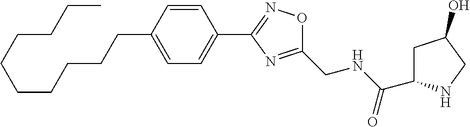 [SMILES: CCCCCCCCCCc1ccc(-c2noc(CNC(=O)[C@@H]3C[C@@H](O)CN3)n2)cc1] |
| 9x | 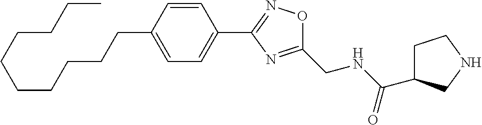 [SMILES: CCCCCCCCCCc1ccc(-c2noc(CNC(=O)[C@H]3CCNC3)n2)cc1] |
| 9y | 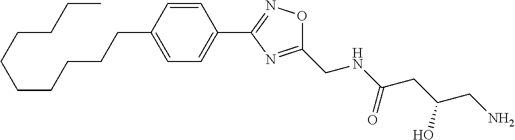 [SMILES: CCCCCCCCCCc1ccc(-c2noc(CNC(=O)C[C@@H](O)CN)n2)cc1] |
| 9z | 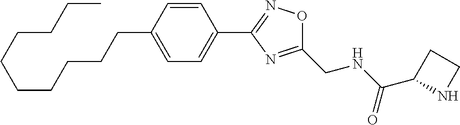 [SMILES: CCCCCCCCCCc1ccc(-c2noc(CNC(=O)[C@@H]3CCN3)n2)cc1] |
| 9aa | 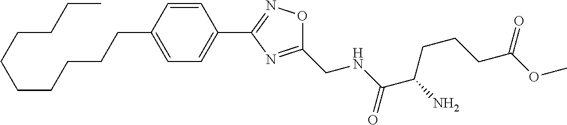 [SMILES: CCCCCCCCCCc1ccc(-c2noc(CNC(=O)[C@@H](N)CCCC(=O)OC)n2)cc1] |
| 9ab | 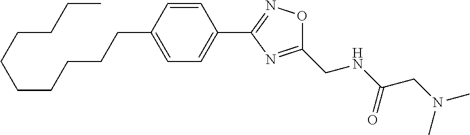 [SMILES: CCCCCCCCCCc1ccc(-c2noc(CNC(=O)CN(C)C)n2)cc1] |
| 9ac | 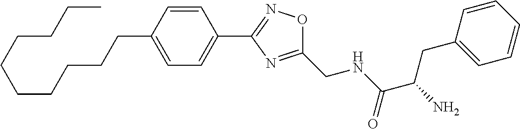 [SMILES: CCCCCCCCCCc1ccc(-c2noc(CNC(=O)[C@@H](N)Cc3ccccc3)n2)cc1] |
| 9ad | 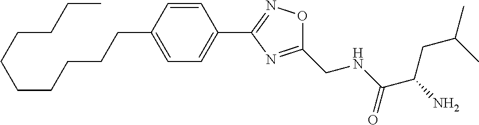 [SMILES: CCCCCCCCCCc1ccc(-c2noc(CNC(=O)[C@@H](N)CC(C)C)n2)cc1] |
| 9ae | 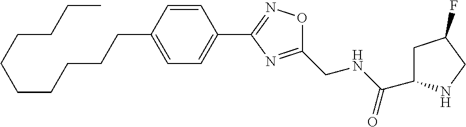 [SMILES: CCCCCCCCCCc1ccc(-c2noc(CNC(=O)[C@@H]3C[C@@H](F)CN3)n2)cc1] |
| 9af | 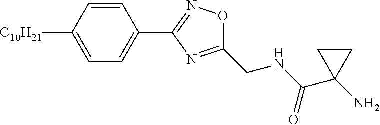 |
| 9ag | 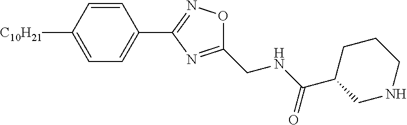 |
| 9ah | 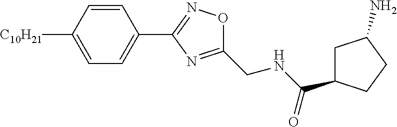 |
| 9ai | 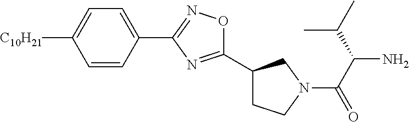 |
| 9aj | 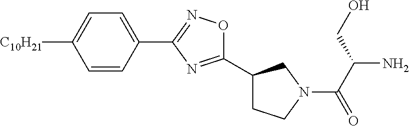 |
| 9ak | 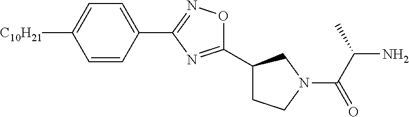 |
| 9al | 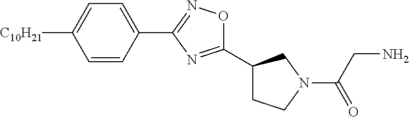 |
| 13a | 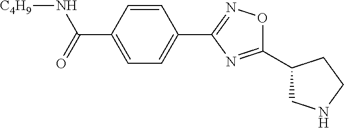 |
| 13b | 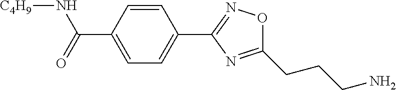 |
| 13c | 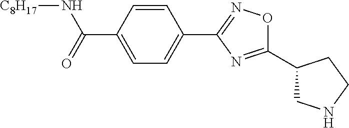 |
| 13d | 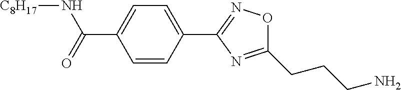 |
| 16a | 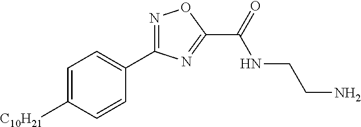 |
| 16b | 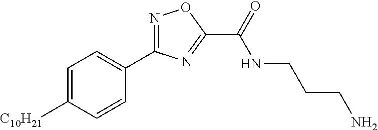 |
| 19a | 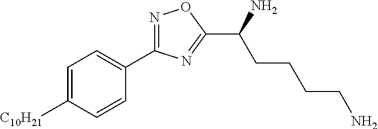 |
| 19b | 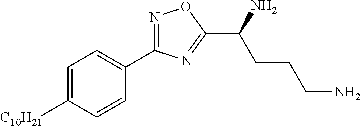 |
| 19c | 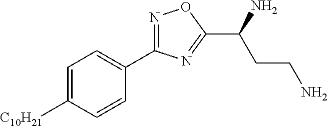 |
| 19d | 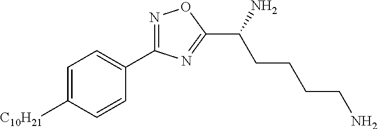 |
| 21 | 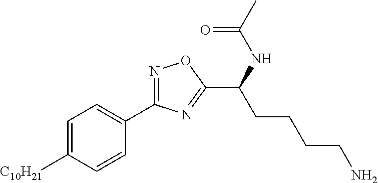 |
| 28 | 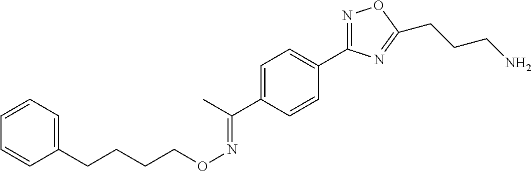 [SMILES: C/C(=N\OCCCCc1ccccc1)c1ccc(-c2noc(CCCN)n2)cc1] |

22. A pharmaceutical composition comprising a compound or pharmaceutically acceptable salt thereof or a pharmaceutically acceptable salt thereof according to claim 1.

23. A method of inhibiting spinster homolog 2 (SPNS2), comprising contacting SPNS2 with an effective amount of a compound or pharmaceutically acceptable salt thereof according to claim 1.

24. The method according to claim 23, wherein the contacting occurs in vivo.

25. A method of treating a patient afflicted by a neoplastic disease, an allergic disease, or an autoimmune disease, comprising administering to the patient a therapeutically effective amount of a compound or pharmaceutically acceptable salt thereof claim 1.

26. The method according to claim 25, wherein
the neoplastic disease is metastatic neoplasms;
the allergic disease is asthma; and the autoimmune disease is multiple sclerosis or comprises one or more progressive forms of multiple sclerosis, type I diabetes, inflammatory bowel diseases, Crohn's disease, ulcerative colitis, Grave's disease, Addison's disease, dermatomyositis, myasthenia gravis, systemic lupus erythematosus, scleroderma, or psoriasis.

27.-32. (canceled)
